(19) United States
(12) Patent Application Publication
(10) Pub. No.: US 2024/0390374 A1
(43) Pub. Date: Nov. 28, 2024

(54) MOCETINOSTAT IN COMBINATION WITH CHEMOTHERAPY TO TREAT RHABDOMYOSARCOMA

(71) Applicant: The Regents of the University of California, Oakland, CA (US)

(72) Inventors: Arun Sayram SINGH, Los Angeles, CA (US); Noah FEDERMAN, Los Angeles, CA (US); Dennis Joseph SLAMON, Woodlands Hills, CA (US); Neil O'BRIEN, Culver City, CA (US)

(73) Assignee: The Regents of the University of California, Oakland, CA (US)

(21) Appl. No.: 18/780,418

(22) Filed: Jul. 22, 2024

Related U.S. Application Data
(63) Continuation of application No. 17/688,603, filed on Mar. 7, 2022, now abandoned.
(60) Provisional application No. 63/158,262, filed on Mar. 8, 2021.

(51) Int. Cl. A61K 31/506; A61K 9/00; A61K 31/395; A61P 35/00

(52) U.S. Cl. CPC A61K 31/506; A61K 9/0053; A61K 31/5545; A61P 35/00

(57) ABSTRACT

The present disclosure provides a method of using mocetinostat in combination with vinorelbine to treating a patient with rhabdomyosarcoma (RMS). The patient can be a child, an adolescent, or an adult having a locally advanced RMS, an unresectable RMS, a metastatic RMS, or a recurrent RMS.

### CROSS-REFERENCE TO RELATED APPLICATION

This application claims benefit of and priority to U.S. Provisional Patent Application Ser. No. 63/158,262, filed Mar. 8, 2021, which is incorporated herein by reference in its entirety.

### FIELD

The present disclosure relates generally to the field of cancer therapy. More specifically, the present disclosure provides mocetinostat in combination with chemotherapy to treat rhabdomyosarcoma.

### BACKGROUND

Rhabdomyosarcoma (RMS) is an aggressive and potentially devastating form of soft tissue cancer that affects children, adolescents and adults. Despite advances in the treatment for RMS, outcomes remain suboptimal. Overall 5-year survival rates are approximately 70-80%. However, patients with metastatic or recurrent disease fare worse. Approximately, 70% of patients who present with metastatic disease and 50-80% of patients who develop recurrent disease, will not survive (Punyko et al., 2005, Pappo et al., 1999 and Winter et al., 2015). Furthermore, the outcomes are worse adults with overall five-year survival of 27% (Sultan et al., 2009). The development of safe and effective treatments for those with metastatic, recurrent, and refractory rhabdomyosarcoma is greatly needed.

RMS has 2 main histologic subtypes: alveolar and embryonal. The embryonal subtype is the most common and has complex genetics without a characteristic gene rearrangement although most have loss of heterozygosity (LOH) at the 11p15 locus. The alveolar subtype has characteristic chromosomal translocations—most often t(2;13)(q35;q14) which fuses PAX2 and FOXO1 or t(1;13)(p36;q14) which fuses PAX7 and FOXO1. Epigenetic regulation, including post-translational modifications of histones, has been shown to play an important role in the pathogenesis of multiple types of cancer (Marks et al., 2001), including rhabdomyosarcoma (Vleeshouwer-Neumann T et al., 2015).

Post-translational modification of histones via acetylation/deacetylation is an important component of epigenetic regulation of gene expression. HDACs are family of enzymes which remove acetyl groups from an —N-acetyl lysine amino acid on histones. De-acetylated histones are positively charged and have higher affinity binding with negatively charged DNA creating a closed chromatin conformation. Thus, HDACs prevent binding of transcription factors, thereby resulting in transcription repression (Glozack and Seto 2007). There are currently 18 known mammalian deacetylase enzymes. HDACs are grouped into Zn2+ dependent HDACs which includes Classes I (HDACs 1-3, and 8), IIa (HDACs 4, 5, 7, and 9), IIb (HDACs 6 and 10) and IV (HDAC11) and NAD+ dependent HDACs which includes class III or sirtuins (SIRT1-7) (Marks et al., 2001).

HDACs have been shown to be involved in initiation and progression of tumorigenesis. They influence cancer initiation through the repression of transcription of tumor suppressor genes, cyclin-dependent kinase inhibitors, and proapoptotic factors which allows for cell proliferation and survival (Glozack and Seto 2007). For example, overexpression of HDAC1 represses the expression of tumor suppressor genes p53 and von Hippel-Lindau and the antiproliferative cyclin-dependent kinase inhibitor p21. HDACs also effect progression of tumorigenesis via regulation of expression of genes involved in angiogenesis including vascular endothelial growth factor (VEGF), and genes involved in migration, invasion, and adhesion thereby allowing for tumor growth and metastasis (Kim et al, 2001 and Glozack and Seto, 2007).

Inhibitors of HDACs have been found to be effective treatments in multiple cancer types. HDAC inhibitors can alter gene expression, including restoring expression of tumor suppressor genes, leading to cell cycle arrest and apoptosis (Glozak et al. 2007). There are a number of HDAC inhibitors including vorinostat, romidepsin, belinostat, and panobinostat that are currently approved for treatment of certain cancers including cutaneous T cell lymphoma and multiple myeloma. However, there are no current approved HDAC inhibitors for RMS. HDAC inhibitors has been shown to inhibit tumor growth and migration in rhabdomyosarcoma cell lines (Keshelava et al. 2009 and Vleeshouweer-Neumann et al, 2015) and are promising potential therapies for RMS.

Mocetinostat is an investigational oral HDAC inhibitor and has orphan drug designation by the FDA for diffuse large B cell lymphoma. Mocetinostat selectively binds to and inhibits HDAC1-3 and 11 with strongest activity on HDAC1. HDAC inhibitors that are non-selective have toxicities which limit the ability to achieve therapeutic dosing. Thus, mocetinostat's selectivity has made it an attractive therapeutic candidate. In in vitro and in vivo studies, it has been shown to have potent anti-proliferative activity with resultant increased p21 expression, the ability to induce cell cycle arrest and apoptosis, and ability to prevent invasion and metastasis (Bonfils et al., 2008; Fournel et al., 2008; Zhou et al., 2008; Zhang et al., 2016).

Thus, there is a need in the development of enhanced and effective treatments for those with metastatic, recurrent, and refractory rhabdomyosarcoma.

### SUMMARY

Rhabdomyosarcoma cells lines have been found to be highly sensitive to mocetinostat, as shown in FIG. 1. In human tumor xenograft models, mocetinostat has demonstrated dose-dependent inhibition of tumor growth (Fournel et al., 2008). Specifically, mocetinostat has also shown efficacy in rhabdomyosarcoma human tumor xenograft models as shown in FIG. 2. Moreover, this figure shows that the combination of chemotherapy with mocetinostat is superior to single agent mocetinostat. Additionally, mocetinostat exposure also leads to differentiation of rhabdomyosarcoma cells are shown by increased relative expression of skeletal muscle markers as shown in FIG. 3.

In one embodiment, the present disclosure provides a method of treating a subject having rhabdomyosarcoma (RMS), comprising the steps of administering mocetinostat to the subject, and administering vinorelbine to the subject. The subject can be a child, an adolescent, or an adult. In one embodiment, the subject is having a locally advanced RMS, an unresectable RMS, a metastatic RMS, or a recurrent RMS. In one embodiment, the mocetinostat is administered to the subject prior to, concurrently, or after administering the vinorelbine to the subject.

In one embodiment, the mocetinostat is administered to the subject orally. In one embodiment, the mocetinostat is administered to the subject at about 40 mg/dose, 70 mg/dose, or 90 mg/dose. In one embodiment, the mocetinostat is administered to the subject more than one time per week. For example, the mocetinostat is administered to the subject three times per week.

In one embodiment, the vinorelbine is administered to the subject intravenously. In one embodiment, the vinorelbine is administered to the subject weekly. In one embodiment, the vinorelbine is administered to the subject at a dose of about 25 mg/m².

In one embodiment, vinorelbine 25 mg/m2 IV is administered on day 1, 8, 15 in combination with mocetinostat 40 mg administered every other day for 9 doses.

In one embodiment, the method or therapeutic combination of mocetinostat and vinorelbine is a synergistic combination.

In one embodiment, a therapeutic combination is provided comprising mocetinostat and vinorelbine. In one embodiment, the therapeutic combination comprises a therapeutically effective dose of mocetinostat and a therapeutically effective dose of vinorelbine.

In one embodiment, a method and therapeutic combination are provided for treating RMS comprising a vinca alkaloid and a HDAC inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows the IC50 for mocetinostat across individual cell lines from different sarcoma subtypes. Rhabdomyosarcoma cell lines are shown in green demonstrating that rhabdomyosarcomas are highly sensitive to mocetinostat.

FIG. 2 shows mocetinostat efficacy in xenograft models of rhabdomyosarcoma. The results show tumor volume change over time in humor tumor xenograft models (derived from patients JR and SJCRH40) exposed to multiple drugs compared to a control. This shows efficacy of mocetinostat (red line) and the combination of mocetinostat and navelbine (also called vinorelbine).

FIG. 3 shows up-regulation of MYBPH, MYH3, MYL1 and TNNC1 by mocetinostat. Shown are the relative expression of MYBPH, MYH3, MYL1, and TNNC1, all of which are markers of skeletal muscle, in vehicle controls (left) versus rhabdomyosarcoma cell lines exposed to mocetinostat. In each panel, left to right, are parental, NT, GFP, FOXO1-KO and PAX3-KO lines.

### DETAILED DESCRIPTION OF THE INVENTION

Multiple clinical trials have evaluated the safety, efficacy, pharmacokinetics, and pharmacodynamics of mocetinostat in humans. Mocetinostat has demonstrated an acceptable safety profile and clinical activity in phase 1 and 2 clinical trials in adult patients with leukemia and lymphoma (Garcia-Manero et al, 2008; Blum et al, 2009; Younes et al, 2011; Batlevi et al., 2017). Phase 1/2 testing in other adult solid tumor patients has shown relatively low toxicity and has established a recommended phase 2 dose (Siu et al., 2008 and Chan et al., 2018).

A phase 1 trial of mocetinostat in adult patients with advanced solid tumors (Siu et al, 2008), reported maximum tolerated dose (MTD)/recommended phase II dose (RP2D) to be 45 mg/m2/day (equivalent to a fixed dose of about 90 mg/day) given orally three times per week for 2 of every 3 weeks. Dose limiting toxicities (DLTs) included fatigue, nausea, vomiting, anorexia, dehydration, hypophosphatemia, and QTc prolongation. These DLTs were seen in 27% of the patients at the 45 mg/m2/day dose. Grade 3 or worse AEs included symptomatic QTc prolongation in one patient that was unlikely to be related to mocetinostat, asymptomatic hypophosphatemia in one patient which resolved with phosphate replacement, fatigue, anorexia, abdominal pain, nausea, vomiting, dehydration and increase in alkaline phosphatase. There were no hematologic AEs.

The two most recent phase 2 trials in solid tumors have been reported by Chan et al., 2018 and Batlevei et al., 2017.

The study by Chan et al., 2018 was a phase 1/2 trial of adult patients with solid tumors of mocetinostat and gemcitabine which found the maximum tolerate dose (MTD)/recommended phase II dose (RP2D) of mocetinostat to be 90 mg three times per week. In the phase 1 portion, DLTs included thrombocytopenia, nausea, vomiting, abdominal pain, diarrhea, fatigue, deep vein thrombosis and mental status change.

Across the phase 1/2 portions of the study, Grade ≥3 treatment-related adverse events (AEs) were reported by 81% of all patients and the most frequent were fatigue (38%), thrombocytopenia (19%), anemia (17%). Of note, included in AEs was that 4% of patients experienced pericardial events (pericardial effusion and cardiac tamponade) considered related to mocetinostat and 26% of patients experienced cystitis or hemorrhagic cystitis considered related to study treatment (mocetinostat and/or gemcitabine). In the phase 2 portion of the study, which was limited to adults with advanced pancreatic cancer, the RP2D of mocetinostat given in combination with gemcitabine was ultimately not well tolerated by the majority of patients and 61% of those patients experienced AEs which resulted in study discontinuation. There were no deaths during study treatment and there was 1 death within the 30-day follow-up period that was due to bronchopneumonia and pulmonary embolism and classified as possibly related to study treatment.

A phase 2 trial of adult patients with follicular lymphoma and diffuse large B cell lymphoma was conducted with 72 patients using starting doses of mocetinostat 70-110 mg three times a week during 4 week cycles (Batlevi et al., 2017). Grade ≥3 treatment-related AEs occurred in 56.9% of patients and the most common were fatigue (23.6%), neutropenia (15.3%) and thrombocytopenia (12.5%). Of note, four patients experienced 5 pericardial events (pericardial effusion, pericarditis, cardiac tamponade) which were considered related to mocetinostat.

Of note, little to no accumulation has been seen with repeated dosing (Garcia et al., 2008; Siu et al., 2008, Boumber et al., 2011). Half-life of elimination is about 10 hours which is longer compared to some of the other HDAC inhibitors. Pharmacokinetic (PK) analyses by Siu et al, 2008 revealed interpatient variability which improved when mocetinostat was coadministered with low pH beverages.

Mocetinostat (N-(2-aminophenyl)-4-(pyridin-3-yl)pyrimidin-2-yl-amino methylbenzamide) is a rationally designed, orally available, Class 1-selective, small molecule, 2-aminobenzamide HDAC inhibitor with potential antineoplastic activity. Mocetinostat binds to and inhibits Class 1 isoforms of HDAC, specifically HDAC 1, 2 and 3, which may result in epigenetic changes in tumor cells and so tumor cell death; although the exact mechanism has yet to be defined, tumor cell death may occur through the induction of apoptosis, differentiation, cell cycle arrest, inhibition of DNA repair, upregulation of tumor suppressors, down regulation of growth factors, oxidative stress, and autophagy, among others.

Vinorelbine (methyl (1R,9R,10S,11R,12R,19R)-11-acetyloxy-12-ethyl-4-[(12S,14R)-16-ethyl-12-methoxycarbonyl-1,10-diazatetracyclo[12.3.1.03,11.04,9]octadeca-3(11),4,6,8,15-pentaen-12-yl]-10-hydroxy-5-methoxy-8-methyl-8,16-diazapentacyclo[10.6.1.01,9.02,7.016,19]nonadeca-2,4,6,13-tetraene-10 carboxylate) is a semi-synthetic vinca alkaloid which has been active as a single agent and part of multi-agent regiments in a number of pediatric malignancies including refractory rhabdomyosarcoma (Casanova et al., 2002, Casanova et al., 2004, Kuttesch et al. 2009, Minard-Colin et al., 2012). The major toxicities seen with vinorelbine are hematologic with neutropenia and anemia. Additional toxicities include mild-moderate nausea and vomiting with <10% with grade II-IV nausea and vomiting, mild to moderate constipation, reversible peripheral neuropathy with grade III-IV neurotoxicity being rare, and hair loss. Sodium valproate has been found to enhance the efficacy of vinorelbine containing treatment regimens in other cancer types (Gavrilov et al., 2012). In some embodiments, the methods and therapeutic combinations disclosed herein further comprise sodium valproate.

In another embodiment, a method and therapeutic combination are provided for treating RMS comprising a vinca alkaloid and a HDAC inhibitor. Non-limiting examples of vinca alkaloids useful for the purposes herein include vinblastine, vincristine, vinflunine and vindesine. These and other vinca alkaloids are known in the art; see, e.g., Moudi et al. 2013, Vinca alkaloids, Int J Prev Med 4 (11): 1231-1235; incorporated herein by reference in its entirety.

Non-limiting examples of HDAC inhibitors useful for the purposes herein include vorinostat, romidepsin, belinostat, and panobinostat. These and other HDAC inhibitors are known in the art; see, e.g., see, e.g., Yoon et al., 2016, HDAC and HDAC Inhibitor: From Cancer to Cardiovascular Diseases, Chonnam Med J. 2016 January; 52 (1): 1-11; Bondarev et al., 2021, Recent developments of HDAC inhibitors: Emerging indications and novel molecules; Brit. J. Chin. Pharm. 87 (12): 4577-4597, each incorporated herein by reference in its entirety. Other non-limiting examples are tacedinaline, entinostat, domatinostat, RG2833, givinostat, KA2507, OBP-801 and AR-42.

In some embodiments, the treating by administering a HDAC inhibitor and administering a vinca alkaloid is synergistic. In some embodiments, the therapeutic combination is a synergistic combination. Any aspects and embodiments of the disclosure herein regarding vinorelbine and mocetinostat are equally applicable to a method and therapeutic composition comprising another vinca alkaloid and another HDAC inhibitor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

As used herein, the terms “comprise”, “comprises”, “comprising”, “includes”, “including”, “having” and their conjugates mean “including but not limited to”.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number, and “ranging/ranges from” a first indicate number “to” a second indicate number, are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of ±5% from the indicated number or range of numbers.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease (e.g., bacterial infection) or prolong the survival of the subject being treated. Determination of a therapeutically effective amount is well within the capability of those skilled in the art.

In one embodiment, for any preparation used in the methods disclosed herein, the therapeutically effective amount or dose can be estimated initially from in vitro assays. For example, a dose can be formulated in animal models and such information can be used to more accurately determine useful doses in humans. In another embodiment, toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on e.g., the subject being treated, the severity of the affliction, the manner of administration, the judgment of the prescribing physician, etc.

A skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including cancer, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test bioassays or systems.

Moreover, suitable doses may also be influenced by permissible daily exposure limits of any compound included in a formulation or method as described herein. Such limits are readily available, including, for example, from industry guidance recommendations provided periodically from the U.S. Food and Drug Administration, and the evaluation of these limits are within the knowledge and understanding of one of ordinary skill in the art.

In one embodiment, a single bolus may be administered. In another embodiment, several divided doses may be administered over time. In yet another embodiment, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

As used herein, the term “administering” refers to bringing in contact with a compound of the present invention. Administration can be accomplished to cells or tissue cultures, or to living organisms, for example humans. In one embodiment, the present invention encompasses administering the compositions of the present invention to a human subject.

In one embodiment, any of the therapeutic or prophylactic drugs or compositions described herein may be administered simultaneously. In another embodiment, they may be administered at different time points. In one embodiment, they may be administered within a few minutes of one another. In another embodiment, they may be administered within a few hours of one another. In another embodiment, they may be administered within 1 hour of one another. In another embodiment, they may be administered within 2 hours of one another. In another embodiment, they may be administered within 5 hours of one another. In another embodiment, they may be administered within 12 of one another. In another embodiment, they may be administered within 24 hours of one another.

In one embodiment, any of the therapeutic or prophylactic drugs or compositions described herein may be administered at the same site of administration. In another embodiment, they may be administered at different sites of administration.

In the description presented herein, each of the steps of the invention and variations thereof are described. This description is not intended to be limiting and changes in the components, sequence of steps, and other variations would be understood to be within the scope of the present invention.

Various embodiments and aspects of the present invention as delineated hereinabove and as claimed in the claims section below find experimental support in the following examples.

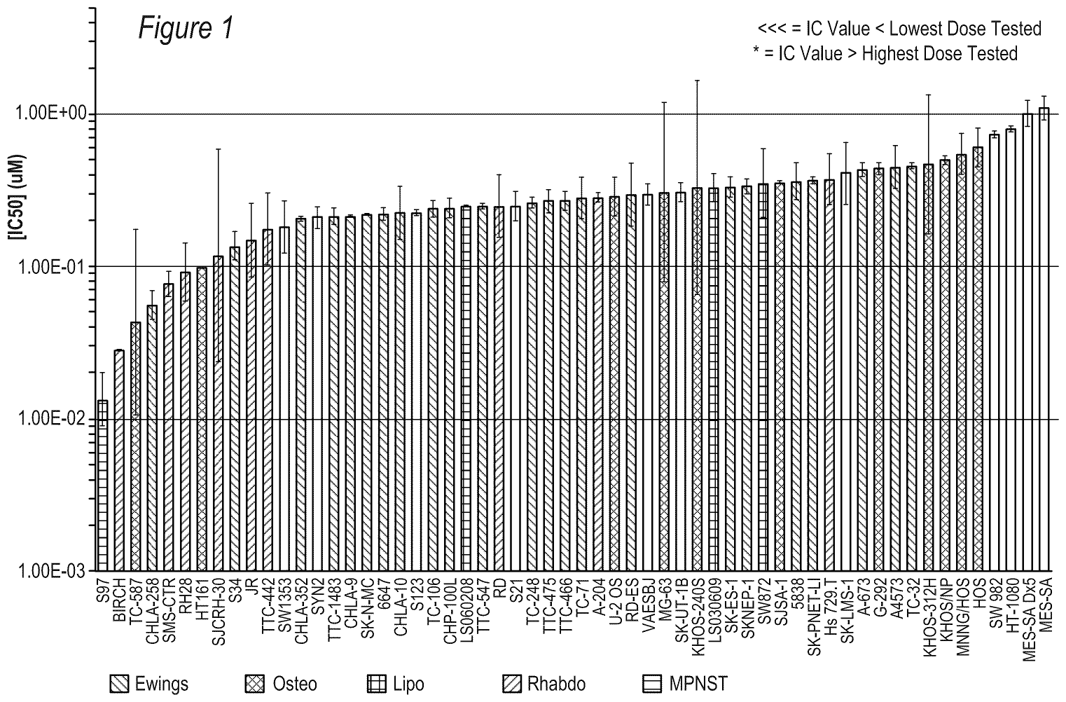

FIG. 1 shows the IC50 for mocetinostat across individual cell lines from different sarcoma subtypes. A subset of cell lines was found to be more sensitive to treatment with mocetinostat and had IC50s well below 100 nM. A large proportion of the sensitive cell lines were found to be rhabdomyosarcomas. Rhabdomyosarcoma cell lines are shown in green demonstrating that rhabdomyosarcomas are highly sensitive to mocetinostat.

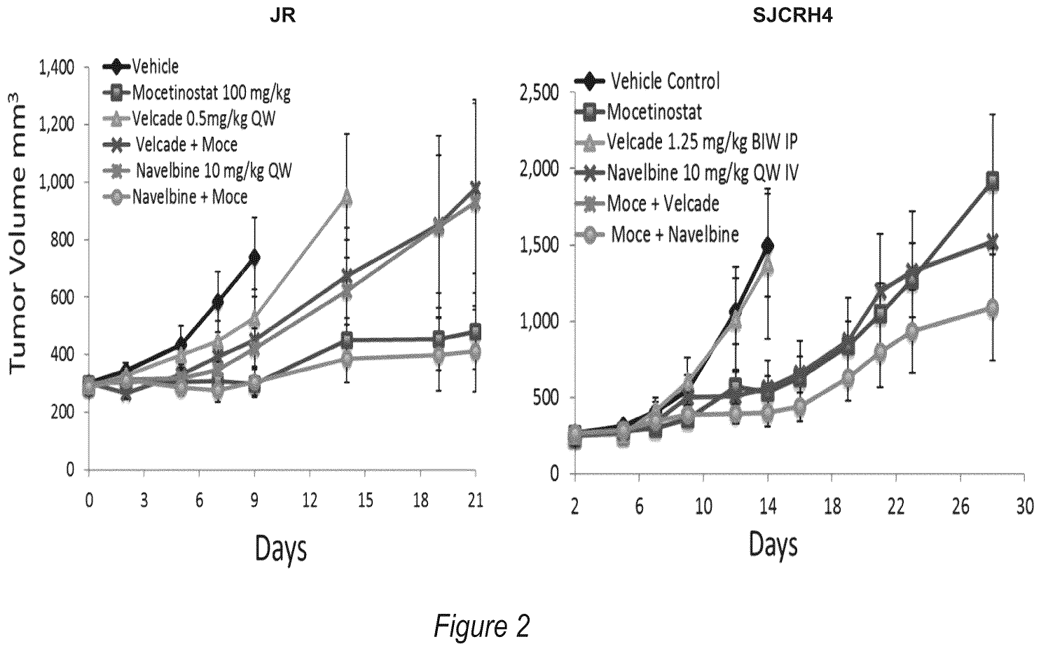

FIG. 2 shows mocetinostat efficacy in xenograft models of rhabdomyosarcoma. The results show tumor volume change over time in humor tumor xenograft models (derived from patients JR and SJCRH40) exposed to multiple drugs compared to a control. This shows efficacy of mocetinostat (red line) and the combination of mocetinostat and navelbine (also called vinorelbine).

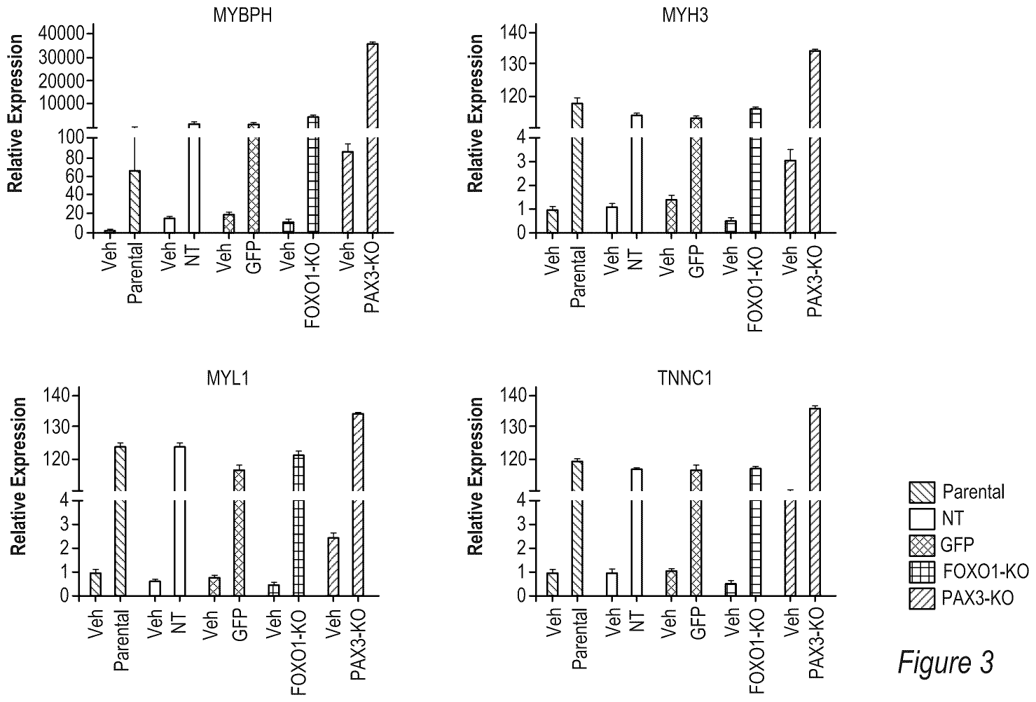

FIG. 3 shows up-regulation of MYBPH, MYH3, MYL1 and TNNC1 by mocetinostat. Shown are the relative expression of MYBPH, MYH3, MYL1, and TNNC1, all of which are markers of skeletal muscle, in vehicle controls (left) versus rhabdomyosarcoma cell lines exposed to mocetinostat.

In one embodiment, a therapeutic combination is provided comprising mocetinostat and vinorelbine. In one embodiment, the therapeutic combination provides greater efficacy than either component when administered alone. In some embodiments, each component of the therapeutic combination is administered independently by a route and dosing regimen (e.g., dose level, doing frequency, duration of dosing, etc.) to provide the maximum benefit to the subject without causing intolerable side effects. In some embodiments, the mocetinostat is administered orally. In some embodiments the vinorelbine is administered intravenously.

In some embodiments, the dose of mocetinostat is 40 mg/dose. In some embodiments, the dose of mocetinostat is 70 mg/dose. In some embodiments, the dose of mocetinostat is 90 mg/dose. In some embodiments, mocetinostat is administered once a week. In some embodiments, mocetinostat is administered twice a week. In some embodiments, mocetinostat is administered three times a week. Any combination of dose and frequency is embraced by this disclosure.

In some embodiments, vinorelbine is administered intravenously. In some embodiments, vinorelbine is administered on days 1, 8, 15 and 21 or a 21 day cycle. In some embodiments, vinorelbine is administered at a dose of 25 mg/m2.

In some embodiments, mocetinostat is administered orally three times a week starting on day 3 for a total of 9 doses for each 21 day cycle, and vinorelbine is administered intravenously via a central venous catheter on days 1, 8 and 15 of each 21 day cycle.

In some embodiments, subjects are treated with mocetinostat for a total of 9 doses per 21 day cycle.

In some embodiments, the dose of mocetinostat is increased over time.

In one embodiment, the mocetinostat is administered to the subject orally. In one embodiment, the mocetinostat is administered to the subject at about 40 mg/dose, 70 mg/dose, or 90 mg/dose. In one embodiment, the mocetinostat is administered to the subject more than one time per week. For example, the mocetinostat is administered to the subject three times per week.

In one embodiment, the vinorelbine is administered to the subject intravenously. In one embodiment, the vinorelbine is administered to the subject weekly. In one embodiment, the vinorelbine is administered to the subject at a dose of about 25 mg/m².

In one embodiment, vinorelbine 25 mg/m2 IV is administered on day 1, 8, 15 in combination with mocetinostat 40 mg administered every other day for 9 doses.

In some embodiments, subjects have refractory RMS. In some embodiments, subjects have recurrent RMS. In some embodiments, subjects have metastatic RMS. In some embodiments, subjects have unresectable RMS. In some embodiments, subjects have any two or more of the foregoing disease criteria.

In some embodiments, the subject has alveolar RMS. In some embodiments, the subject has embryonal RMS.

In some embodiments, the subject is a child. In some embodiments the subject's age is below about 18 years of age. In some embodiments, the subject is an adolescent. In some embodiments, the subjects age is between about 12 and 80 years. In some embodiments, the subject is an adult. In some embodiments, the subject is greater or equal to about 12 years of age.

In some embodiments, the treatment shows an improvement in any one or more of the efficacy endpoints described herein. In some embodiments, the treatment shows an improvement in any one or more of the secondary endpoints described herein.

In some embodiments, the treatment shows an improvement in any one or more of the primary objectives described herein. In some embodiments, the treatment shows an improvement in any one or more of the secondary objectives described herein. In some embodiments, the treatment shows an improvement in any one or more of the primary objectives, secondary objectives, secondary endpoints, or efficacy endpoints as described herein. In some embodiments, the treatment shows an improvement in any one or more of the secondary objectives described herein. In some embodiments, the treatment shows an improvement in any two or more of the primary objectives, secondary objectives, secondary endpoints, or efficacy endpoints as described herein. In some embodiments, the treatment shows an improvement in any one or more of the secondary objectives described herein. In some embodiments, the treatment shows an improvement in any three or more of the primary objectives, secondary objectives, secondary endpoints, or efficacy endpoints as described herein. In some embodiments, the treatment shows an improvement in any one or more of the secondary objectives described herein. In some embodiments, the treatment shows an improvement in any four or more of the primary objectives, secondary objectives, secondary endpoints, or efficacy endpoints as described herein.

In some embodiments, the treatment shows decreased size of the RMS by radiologic imaging. In some embodiments, the treatment shows decreased metabolic uptake of fluorodeoxyglucose (FDG) PET radiotracer by whole body PET/CT. In some embodiments, the treatment shows decreased size of the RMS by radiologic imaging and decreased metabolic uptake of FDG PET radiotracer by whole body PET/CT.

In some embodiments, treatment reduces standardized uptake value (SUV) of the tumor measured by PET scan.

In some embodiments, improvement in objective tumor response is observed, as measured using the Response Evaluation Criteria in Solid Tumors (RECIST version 1.1) in subjects with solid tumors. In some embodiments, improvement in PFS is observed, defined as time from first dose of vinorelbine to tumor progression or death due to any cause. In some embodiments, improvement in Disease Control (DC) is observed, defined as the proportion of subjects with a confirmed CR, PR, or SD according to RECIST v1.1. In some embodiments, improved Duration of Response (DOR) is observed, as defined from the first date a response is identified (either CR or PR) until the date of disease progression. In some embodiments, any two or more of the foregoing outcomes are observed. In some embodiments, any three or more of the foregoing outcomes are observed.

In some embodiments, treated subjects show an improved clinical benefit rate (CBR) compared to a control group of subjects or reference population of subjects (e.g., have not undergone treatment with the combination disclosed herein). In some embodiments, the reference population may be subjects' responses before receiving the therapeutic combination disclosed herein. In one embodiment, CBR comprises patients having a complete response plus patients having a partial response and stable disease. In some embodiments, treated subjects have an improved antitumor activity as measured by overall response rate (ORR), duration of response (DOR), disease control (DC), duration of disease control, or any combination thereof, as compared to a reference population. In some embodiments, ORR includes Complete Response (CR) and Partial Response (PR). In some embodiment, DOR as defined from the first date a response is identified (either CR or PR) until the date of disease progression. In some embodiments, Disease Control (DC) defined as the proportion of subjects with a confirmed CR, PR or Stable Disease (SD). In some embodiments, Duration of Disease Control defined as first date of disease control identified (either CR, PR or SD) until the date of progression. In some embodiments, PFS as defined by time from first dose of vinorelbine to tumor progression or death due to any cause. In some embodiments, any two or more of the foregoing outcomes are observed. In some embodiments, any three or more of the foregoing outcomes are observed.

In some embodiments, subjects have an improved PFS compared to a reference population.

In some embodiments, treated subjects have an improved response as measured by exploratory biomarkers than those of a reference population.

In some embodiments, dosing is administered or continued in a subject, or treatment cycles continued, with any one or more of the following criteria: ANC >1000/mm3, hemoglobin >8.0 mg/dL, platelets >50000/mm3, or non-hematological toxicity recovered to <Grade 1 or tolerable Grade 2).

In some embodiments, the disease control rate/clinical benefit rate (CR+PR+SD) is 100%. In some embodiments, the disease control rate/clinical benefit rate (CR+PR+SD) is ≥95%. In some embodiments, the disease control rate/clinical benefit rate (CR+PR+SD) is ≥90%. In some embodiments, the disease control rate/clinical benefit rate (CR+PR+SD) is ≥85%. In some embodiments, the disease control rate/clinical benefit rate (CR+PR+SD) is ≥80%. In some embodiments, the disease control rate/clinical benefit rate (CR+PR+SD) is ≥75%. In some embodiments, the disease control rate/clinical benefit rate (CR+PR+SD) is ≥70%. In some embodiments, the disease control rate/clinical benefit rate (CR+PR+SD) is ≥65%. In some embodiments, the disease control rate/clinical benefit rate (CR+PR+SD) is ≥60%. In some embodiments, the disease control rate/clinical benefit rate (CR+PR+SD) is ≥55%. In some embodiments, the disease control rate/clinical benefit rate (CR+PR+SD) is ≥50%.

In some embodiments, a rapid and durable response is observed in 100% of subjects. In some embodiments, a rapid and durable response is observed in ≥95% of subjects. In some embodiments, a rapid and durable response is observed in ≥90% of subjects. In some embodiments, a rapid and durable response is observed in ≥85% of subjects. In some embodiments, a rapid and durable response is observed in ≥80% of subjects. In some embodiments, a rapid and durable response is observed in ≥75% of subjects. In some embodiments, a rapid and durable response is observed in ≥70% of subjects. In some embodiments, a rapid and durable response is observed in ≥65% of subjects. In some embodiments, a rapid and durable response is observed in ≥60% of subjects. In some embodiments, a rapid and durable response is observed in ≥55% of subjects. In some embodiments, a rapid and durable response is observed in ≥50% of subjects.

In some embodiments, the adverse events related to treatment are: for leukopenia, less than or equal to grade 1, grade 2 or grade 3; for anemia, less than or equal to grade 1, grade 2 or grade 3; for lymphocytopenia, less than or equal to grade 1 or grade 2; for low calcium, less than or equal to grade 1; for nausea, less than or equal to grade 1 or grade 2; for bloating, less than or equal to grade 1; for neutropenia, less than or equal to grade 1, grade 2, grade 3 or grade 4; for constipation, less than or equal to grade 1; for elevated AST, less than or equal to grade 1; for elevated ALT, less than or equal to grade 1.

In some embodiments, the subject meets one or more inclusion criteria described in Example 1 or Example 2. In some embodiments, the subject meets all inclusion criteria described in Example 1 or Example 1. In some embodiments, the subject does not have one or more exclusion criteria described in Example 1 or Example 2. In some embodiments, the subject does not have any of the exclusion criteria described in Example 1 or Example 1.

In some embodiments, the subject has a FOXO translocation. In some embodiments the subject does not have a FOXO translocation.

In some embodiments, a modified intent to treat (mITT) approach is used for efficacy analysis, in which the mITT population will consist of all subjects who receive any amount of study drug. In some embodiments, tumor response rates are summarized by dose group and for all subjects who receive the RP2D, including those from the dose escalation and expansion phases. In some embodiments, responses are classified as CR, PR, SD or PD according to RECIST v1.1 criteria. In some embodiments, summaries will be based on the best response recorded up until disease progression. In some embodiments, subjects who discontinue prior to the first 6-weekly response assessment will be considered as non-responders in the primary efficacy analysis. In some embodiments, objective tumor response (CR or PR) is summarized, as will PFS, OS, DCR and duration of response and DCR. In some embodiments, time to event data will be summarized by Kaplan-Meier methods, including 25th, 50th (median) and 75th percentiles with point estimates and two-sided 95% confidence intervals, as well as number and percent of censored observations. In some embodiments, the treated subject exhibits an improvement in any one or more efficacy criteria. In some embodiments, the treated subject exhibits an improvement in any one or more efficacy criteria as compared to a reference population.

In some embodiments, treating RMS is provided by a therapeutically effective synergistic combination of mocetinostat and vinorelbine. In some embodiments, the therapeutically effective combination is a synergistic combination. In some embodiments, the methods and therapeutic combinations disclosed herein provide a synergistic effect, e.g., the therapeutic benefit of the combination is greater than the sum of the therapeutic benefit on RMS of each component administered in the absence of the other. In some embodiments, the efficacy of either drug administered alone is low or absent when not administered in combination with the other drug. Such synergy may be achieved, in certain embodiments, by titrating the dose level and dosing frequency (i.e., the dosing regimen) of each component of the combination individually. In some embodiments, the lower dose level or dose frequency of one or both component elicits fewer side effects.

In another embodiment, a method and therapeutic combination are provided for treating RMS comprising a vinca alkaloid and a HDAC inhibitor. Non-limiting examples of vinca alkaloids useful for the purposes herein include vinblastine, vincristine and vindesine. These and other vinca alkaloids are known in the art. Non-limiting examples of HDAC inhibitors useful for the purposes herein include vorinostat, romidepsin, belinostat, and panobinostat. These and other HDAC inhibitors are known in the art. In some embodiments, the treating by administering a HDAC inhibitor and administering a vinca alkaloid is synergistic. In some embodiments, the therapeutic combination is a synergistic combination. Any aspects and embodiments of the disclosure herein regarding vinorelbine and mocetinostat are equally applicable to a method and therapeutic composition comprising another vinca alkaloid and another HDAC inhibitor.

The following examples provide protocols for evaluating the safety and efficacy of a combination of mocetinostat and vinorelbine. Any and all aspects of the protocols such as but not limited to patient selection criteria, inclusion criteria, exclusion criteria, assessment of primary and secondary endpoints, assessments of safety, among other aspects of the protocol are fully embraced herein.

### Example 1

A Phase I/II Dose Escalation/Expansion Clinical Trial of Mocetinostat in Combination with Vinorelbine in Children, Adolescents and Young Adults with Refractory and/or Recurrent Rhabdomyosarcoma

Study period: 18-30 months Clinical phase: I/II

Objectives—Phase 1 Dose Escalation Cohorts: Primary Objective:
The primary objective of the Phase 1 dose escalation of mocetinostat segment is to determine the first cycle dose-limiting toxicities (DLTs), maximum tolerated dose (MTD), and a biologically effective and recommended Phase 2 dose (RP2D) of mocetinostat administered orally three times per week starting on day 3 of each cycle for a total of 9 doses per 21 day cycle given in combination with vinorelbine administered intravenously on days 1, 8, 15 of a 21 day cycle in subjects with refractory or recurrent RMS.

### Secondary Objectives:

Safety profile of Mocetinostat in combination with vinorelbine as characterized by Adverse Event (AE) type, severity, timing and relationship to study drugs, as well as laboratory abnormalities in the first and subsequent treatment cycles
Pharmacokinetics (PK) of Mocetinostat in plasma
Clinical benefit rate (CBR)=complete response (CR)+partial response (PR) and stable disease (SD)) of mocetinostat+vinorelbine in metastatic/refractory/unresectable RMS according to RECIST v1.1.
Antitumor activity of mocetinostat+vinorelbine in refractory/recurrent RMS as measured by Overall Response Rate (ORR), Duration of Response (DOR), Disease Control (DC), Duration of Disease Control, as well as Progression-Free Survival (PFS) according to RECIST v1.1.
ORR includes Complete Response (CR) and Partial Response (PR).
DOR as defined from the first date a response is identified (either CR or PR) until the date of disease progression
Disease Control (DC) defined as the proportion of subjects with a confirmed CR, PR or Stable Disease (SD).
Duration of Disease Control defined as first date of disease control identified (either CR, PR or SD) until the date of progression
PFS as defined by time from first dose of vinorelbine to tumor progression or death due to any cause
Pharmacodynamics of mocetinostat on molecular targets in surrogate tissue.
Exploratory biomarker development to enable prediction of drug toxicity, tumor response and the mechanism(s) of acquired study drug resistance.
Obtain RMS tissue biological samples pre-treatment and at progression to assess for differences in gene expression by Next Gen Sequencing and RNA Seq.

### Objectives—Phase 2 Expansion Cohort:

### Primary Objective:

The primary objective of the expansion cohort is to determine the PFS, defined as time from first dose of vinorelbine to tumor progression or death due to any cause, at the RP2D of mocetinostat administered orally three times per week starting on day 3 for a total of 9 doses per 21 day cycle given in combination with vinorelbine on days 1, 8, 15 of a 21 day cycle in subjects with refractory or recurrent RMS.

### Secondary Objectives:

Determine PFS at 4 months and 6 months.
Antitumor activity of mocetinostat+vinorelbine in metastatic/refractory RMA as measured by Overall Response Rate (ORR) and Duration of Response (DOR), Disease Control (DC), Duration of Disease Control, as well as Progression-Free Survival (PFS) according to RECIST v1.1
ORR includes Complete Response (CR) and Partial Response (PR).
DOR as defined from the first date a response is identified (either CR or PR) until the date of disease progression
Disease Control (DC) defined as the proportion of subjects with a confirmed CR, PR or Stable Disease (SD)
Duration of Disease Control defined as first date of disease control identified (either CR, PR or SD) until the date of progression
Safety and tolerability of mocetinostat and vinorelbine as characterized by Adverse Event type, severity, timing and relationship to study drug, as well as laboratory abnormalities
Pharmacodynamics of mocetinostat on molecular targets in surrogate tissue.
Exploratory biomarker development to enable prediction of drug toxicity, tumor response and the mechanism(s) of acquired study drug resistance.
Obtain RMS tissue biological samples pre-treatment and at progression to assess for differences in gene expression by Next Gen Sequencing and RNA Seq.

### Study Design:

RMS001 is a single center, open-label, Phase 1/2 study in which the safety and efficacy of mocetinostat in combination with vinorelbine will be evaluated in subjects with locally advanced/unresectable RMS or metastatic or recurrent RMS who have failed front line therapies. Once an appropriate subject has been identified, a 30-day screening period will begin to evaluate eligibility using the defined study inclusion and exclusion criteria.

Vinorelbine will be administered at a dose of 25 mg/m2 given intravenously on days 1, 8, and 15 of a 21 day cycle.

Mocetinostat will be administered orally three times per week beginning on day 3 for a total of 9 doses in a 21 day cycle.

### Phase 1 Dose Escalation Cohorts:

The starting daily dose level for mocetinostat in the dose escalation segment will be 40 mg per dose for cohort 1. Cohort 2 dosing will be mocetinostat 70 mg per dose. Cohort 3 dosing will be mocetinostat 90 mg per dose.

TABLE 1
Phase 1 Dose Escalation Cohort Mocetinostat Dosing:

| Cohort | Mocetinostat Dose |
|---|---|
| Cohort 1 | 40 mg/dose |
| Cohort 2 | 70 mg/dose |
| Cohort 3 | 90 mg/dose |

A “3+3” subject enrollment scheme will be followed during the dose escalation. This segment will be performed in sequential cohorts of subjects receiving mocetinostat orally. Cycles will consist of vinorelbine treatment once daily on days 1, 8 and 15 and mocetinostat treatment three times per week beginning on day 3 for a total of 9 doses in a 21 day cycle. If 2 of 3 subjects experience a first-cycle DLT then accrual to the cohort will cease. If a first-cycle DLT is seen in 1 of the 3 subjects in a cohort, that cohort will enroll an additional 3 subjects. The dose escalation will continue until a first cycle DLT has been observed in 2 of 3 or 2 of 6 subjects. DLT is defined as an adverse event occurring during the first cycle that is at least possibly related to mocetinostat and meets the DLT definition outlined in Section 4, Study Design. When 0 of 3, or 1 of 6 subjects in a cohort experience DLT, the dose will be escalated in the subsequent cohort.

Dose escalation will begin with dose increase in successive cohorts of 3 subjects until 1 subject experiences a first-cycle DLT (as defined in Section 4, Study Design); or 2 subjects experience similar AEs that are greater than or equal to grade 2 severity (grade 3 severity for hematological AEs) which occur during the first cycle. A toxicity that is clearly and incontrovertibly unrelated to mocetinostat may be excluded in consultation with the Primary Investigators (PIs).

Once this predetermined toxicity level has been encountered, subsequent cohorts will dose escalate following review of AEs and discussion with the investigators.

The MTD is the dose level at which 0 of 6 or 1 of 6 subjects experience first-cycle DLT, and at least 2 of 3 or 2 of 6 subjects experience first-cycle DLT at the next higher dose level. Effectively, the MTD is the highest dose associated with first-cycle DLT in <33% of subjects.

It is anticipated that there will be 3 cohorts enrolled during the dose escalation segment of this trial.

After the RP2D has been determined, an expansion cohort will be enrolled in the phase 2 portion of the study.

### Phase 2 Expansion Cohort:

The expansion cohort segment of this study will consist of 20 additional subjects with refractory alveolar RMS >12 years of age. All subjects in the expansion cohort will receive the RP2D of Mocetinostat.

Dose escalation and de-escalation in Phase 1 Dose Escalation and Phase 2 Dose Expansion Cohorts:

There will be no intra-patient dose escalation of vinorelbine or mocetinostat in the Phase 1 Dose Escalation and Phase 2 Dose Expansion Cohorts.

Additional cycles of therapy may be administered provided that the subject meets the following criteria on Day 1 of each cycle:
ANC>1,000/mm3.
Hemoglobin >8.0 gm/dL (Blood transfusions are permitted).
Platelets >50,000/mm3
Non-hematologic toxicity recovered to <Grade 1 (or tolerable Grade 2)

Subjects with toxicities that are manageable with supportive therapy may not require dose reductions (e.g., nausea/vomiting may be treated with antiemetics, anemia may be treated with blood transfusions).

Dose de-escalation for subjects may be warranted after Cycle 1 as a consequence of drug-related toxicities. Dose reduction will be documented in the CRF along with reason for reduction. In the Phase 1 Dose Escalation Cohorts, mocetinostat dose de-escalation of 1-2 levels is allowed but there will be no dose de-escalation below Cohort 1 level dosing of 40 mg. If unacceptable toxicity occurs at the cohort 1 dosing, subjects will be instructed to hold or discontinue treatment. In the Phase 2 Dose Expansion Cohorts, mocetinostat dose de-escalation of 1-2 levels below the dose chosen for this phase is allowed unless the Cohort 1 (40 mg) dose is chosen. At this point, if a subject experiences unacceptable toxicity, they will be instructed to hold or discontinue treatment. Doses reduced for drug-related toxicity should not be re-escalated, even if there is minimal or no toxicity with the reduced dose.

For adverse events not specified below, doses may be reduced or held at the discretion of the investigator for the subject's safety. The sponsor should be made aware of such reductions. Recommended dose modifications based on type of AE or laboratory findings:

TABLE 2
Recommended dose modifications based
on type of AE or laboratory findings:

| AE or lab finding | Dose modification |
|---|---|
| ≥Grade 2 | Subjects experiencing ≥Grade 2 neutropenia |
| neutropenia | may receive G-CSF or other myeloid growth |
| | factors after the first cycle or as defined |
| | in the dose-escalation plan. |
| Grade 3 or 4 | First occurrence-hold mocetinostat and |
| neutropenia | vinorelbine until ANC >1,000/mm³, then |
| | resume mocetinostat and vinorelbine at |
| | same dose. |
| | Second occurrence-hold mocetinostat and |
| | vinorelbine until ANC >1,000/mm3, then |
| | reduce mocetinostat dose to one dose cohort |
| | level lower than the current dose and |
| | resume vinorelbine at same dose |
| | Dose reduction for neutropenia should occur |
| | when the next cycle of study drug is begun. |
| Grade 4 | Any occurrence despite use of G-CSF or |
| neutropenia lasting | other myeloid growth factors-hold |
| longer than 7 days | mocetinostat and vinorelbine until |
| | ANC >1,000/mm3, then reduce mocetinostat |
| | dose to one dose cohort level lower than |
| | the current dose and resume |
| Grade 3 or 4 | Any occurrence despite use of G-CSF or |
| febrile | other myeloid growth factors-hold |
| neutropenia | mocetinostat and vinorelbine until |
| | ANC >1,000/mm3 and temperature <38 |
| | degrees Celsius, reduce mocetinostat |
| | dose to one dose cohort level lower than |
| Grade 3 or 4 | First occurrence-hold mocetinostat and |
| thrombocytopenia | vinorelbine until platelets ≥50,000/mm3, |
| | then resume mocetinostat and vinorelbine |
| | at same dose. |
| | Second occurrence-hold mocetinostat and |
| | vinorelbine until platelets ≥50,000/mm3, |
| | then reduce mocetinostat dose to one dose |
| | cohort level lower than the current dose |
| | and resume vinorelbine at the same dose. |
| Hemoglobin <8.0 | Any occurrence-hold mocetinostat and |
| gm/dL | vinorelbine until hemoglobin >8.0 gm/dL, |
| | then resume mocetinostat and vinorelbine |
| | at same dose. |
| | Blood transfusions are permitted. |
| Grade 2 or greater | Any occurrence-hold mocetinostat and |
| non-hematologic | vinorelbine until toxicities have resolved |
| toxicity (unless | or improved to Grade 2 severity levels, |
| clearly and | then resume mocetinostat and vinorelbine |
| incontrovertibly | at same dose if event was a tolerable Grade |
| unrelated to | 2 (at PI's discretion). Reduce mocetinostat |
| mocetinostat): | dose to one dose cohort level lower than the |
| | current dose if event is Grade 3 or 4 or |
| | intolerable Grade 2 in severity. |
| Management of | In the event of symptoms of cystitis (e.g. |
| Mocetinostat | dysuria, pollakiuria, hematuria, urgency, |
| Associated Cystitis | or bladder spasm) suspected to be attributable |
| | to mocetinostat treatment: |
| | Perform diagnostic evaluation and manage per |
| | institutional standards |
| | If clinically significant symptoms persist |
| | despite a negative diagnostic assessments or |
| | treatment of an associated condition, interrupt |
| | study treatment until resolution of clinically |
| | significant symptoms; and Resume dosing of |
| | mocetinostat when medically appropriate, |
| Management of | Patients will be assessed for evidence of |
| Mocetinostat in Event | pericardial toxicity during scheduled visits |
| of Pericardial Toxicity | according to the Schedule of Assessments |
| | The following findings would heighten suspicion |
| | of pericardial effusion or pericarditis and |
| | prompt immediate evaluation by ECHO: |
| | Clinical exam: hypotension, jugular venous |
| | distension, pulsus paradoxus, faint heart |
| | sounds, friction rub, and/or arrhythmia |
| | ECG: sinus tachycardia, atrial fibrillation, |
| | atrial flutter, low voltage with nonspecific |
| | ST-T wave changes and ST elevation or PR |
| | depressions, arrhythmia |
| | ECHOs will be used to assess and categorize |
| | pericardial fluid as minimal (or trivial), |
| | small, moderate or large and will assess for |
| | hemodynamic compromise. |

TABLE 3
Pericardial Effusion and Patient Management Guidelines

| Category | Definitions | Patient Management |
|---|---|---|
| Minimal | A small echo-free | De novo (i.e., not |
| (or trivial) | space in the posterior | present at baseline) |
| | atrioventricular groove | pericardial effusion: |
| | that is visible only | Study treatment may |
| | in systole when the | be continued at the |
| | heart has pulled away | discretion of the |
| | from the pericardium. | investigator. |
| | Typically represents | Increased ECHO and |
| | a normal amount of | ECG monitoring weekly |
| | pericardial fluid in | until effusion is no |
| | a disease-free state. | longer present or has |
| | | not progressed over a |
| | | period of 2 weeks. |
| | | Regular assessment |
| | | schedule afterwards. |
| Small | <1 cm of posterior | Study drug will not |
| | echo-free space, with | be discontinued in |
| | or without fluid | these Patients, at the |
| | accumulation elsewhere, | discretion of the |
| | present throughout the | investigator, unless |
| | cardiac cycle, including | the effusion progresses. |
| | diastole (and not only | Increased ECHO and |
| | systole). | ECG monitoring weekly |
| | | for the first month |
| | | after the new effusion |
| | | first noted or until |
| | | the effusion has |
| | | regressed (if sooner). |
| | | Treatment for the |
| | | effusion may be |
| | | administered at the |
| | | discretion of the |
| | | Investigator. |
| Moderate | 1 to 2 cm of echo- | Remove immediately |
| | free space. Moderate | from study treatment. |
| | effusions tend to be | Manage according to |
| | seen along the length | the standard of care |
| | of the posterior wall | at the discretion of |
| | but not anteriorly. | the investigator. |
| Large | >2 cm of maximal | Refer to cardiologist |
| | separation. Large | for follow-up as |
| | effusions tend to be | clinically indicated, |
| | seen circumferentially. | until resolution of |
| | | stabilization. |
| Hemodynamic | RV compression, IVC | Remove immediately |
| Compromise | dilation without respiratory | from study treatment. |
| | variation, abnormal flow | Refer to cardiologist |
| | variation across the AV | for follow up as |
| | valves without respiratory | clinically indicated, |
| | variation, enlarged or | until resolution or |
| | collapsed ventricles. | stabilization. |
| | RA diastolic collapse | Collect blood and test |
| | in isolation is too | for anti-nuclear |
| | non-specific to signal | antibody (ANA) and |
| | hemodynamic compromise, | anti-histone antibody |
| | but should be considered | |
| | consistent with this | |
| | diagnosis when | |
| | accompanied by other | |
| | findings | |

In exceptional circumstances where ECHO is not considered a technically optimal assessment of pericardial space (e.g., overweight patient), other methods (e.g., MRI) should be used for pericardial assessments. In such cases, the guidelines provided in Table 3 would not apply, and the evaluation should be performed in consultation with the Sponsor. In the event that a pericardial effusion is first identified by a method other than ECHO, efforts should be made to obtain an ECHO for assessment of effusion size.

Adverse event (AE) monitoring for subjects will begin upon the initiation of vinorelbine and will continue for 28 days after the last administration of mocetinostat. AEs will be graded according to the National Cancer Institute Common Terminology Criteria for Adverse Events Version 5.0 (NCI CTCAE v5.0). For events not reported in the CTCAE, the Investigator will use the grade or adjectives as defined in the Adverse Events section of the study protocol.

Subjects will have tumor assessment performed approximately every 6 weeks (+/−1 week), beginning from the initiation of vinorelbine. Tumor assessments will cease if the subject begins a different cancer therapy or withdraws consent. An End of Treatment Visit will be conducted within 7 days after the end of the last cycle. A Safety Follow-Up telephone call will be conducted approximately 28 days following the last day of mocetinostat. Safety will be monitored via laboratory assessments, physical examinations, electrocardiograms (ECG), vital signs, and AEs. Study assessments will be performed as per Schedule of Assessments (see Tables at end of the synopsis).

### Number of Subjects (Planned and Analyzed):

Approximately 18 subjects may be enrolled in the dose escalation portion of the study (3 cohorts). Approximately 20 subjects will be enrolled in the expansion cohort.

### Inclusion Criteria:

Subjects who meet the following inclusion criteria will be eligible to participate in this study:
1. Ages 13-35 years old
2. Willing and able to provide written IRB/IEC-approved Informed Consent. For subjects <18 years of age, their parents or legal guardians must sign a written informed consent. Assent, when appropriate, will be obtained according to institutional guidelines.
3. Have histologically or cytologically confirmed diagnosis of Rhabdomyosarcoma with locally advanced/unresectable, metastatic, refractory or relapsed disease who have failed standard therapy and for whom no known curative therapy exists.
4. Measurable disease according to RECIST version 1.1.
5. Prior cancer therapy: Subjects may have received any number of prior therapy regimens. In the Investigator's opinion, subjects must have tolerated prior cytotoxic therapies well and have adequate bone marrow reserve. At the time of treatment initiation, at least 3 weeks must have elapsed after prior cytotoxic chemotherapy. At least 7 days must have elapsed since completion of any prior non-cytotoxic cancer therapy and any associated AEs must have resolved.
6. Prior radiotherapy is allowed if ≥2 weeks have elapsed for local palliative XRT (small port); ≥6 months must have elapsed if prior total body irradiation, craniospinal XRT or if >50% radiation of the pelvis; >6 weeks must have elapsed if other substantial bone marrow radiation (defined per PI's discretion). Subjects who have received brain irradiation must have completed whole brain radiotherapy and/or gamma knife at least 4 weeks prior to enrollment.
7. Subjects with controlled asymptomatic CNS involvement which has not required treatment in >/=28 days are eligible.
8. Resolution of all acute toxic effects (excluding alopecia) of any prior anti-cancer therapy to NCI CTCAE (Version 4.03) Grade <1 or to the baseline laboratory values as defined in the table below.
9. Eastern Cooperative Oncology Group (ECOG) performance status (PS)≤2 in subjects ≥17 years old; or Karnofsky/Lansky >50 in subjects <16 years old.
10. Subjects age >18 years for phase 1 dose escalation cohort. Subjects must be >12 years old for the phase 2 dose expansion cohort.
11. Life expectancy of at least 3 months.
12. Baseline laboratory values fulfilling the following requirements:

| | |
|---|---|
| Absolute Neutrophil Count | ≥1000/mm3 (≥1.0 × 109/L) |
| (ANC) | |
| Platelets (PLT) | ≥100,000/mm3 (≥100 × 109/L) |
| | (transfusion independent, defined |
| | as not receiving platelet transfusions |
| | within a 7 day period prior to screening) |
| Hemoglobin >9.0 g/dL | >9.0 g/dL (transfusions are allowed) |
| (transfusions are allowed) | |
| Serum Creatinine | ≤1.5 × ULN |
| Or Creatinine | >60 mL/min |
| Clearance | |
| Total Serum Bilirubin | ≤1.5 × ULN ≤5 × ULN if Gilbert' sSyndrome |
| Liver Transaminases | ≤2.5 × ULN; ≤5 × ULN if liver |
| (AST/ALT) | metastases are present |
| Pregnancy test if female | Negative within 7 days of starting treatment |
| of child-bearing potential | |

AST/ALT = aspartate aminotransferase/alanine aminotransferase, ULN = upper limit of normal
Growth factor(s): Growth factors that support platelet or white cell number or function must not have been administered within the 7 days prior to screening.

13. Cardiac ejection fraction >50% or shortening fraction >28% by ECHO or MUGA.
14 Females of child-bearing potential must have a negative pregnancy test during screening and be neither breastfeeding nor intending to become pregnant during study participation. Females of childbearing potential must agree to avoid pregnancy during the study and commit to abstinence from heterosexual intercourse or agree to use two methods of birth control (one highly effective method and one additional effective method) at least 4 weeks before the start of protocol therapy, for the duration of study participation, and for 6 months after the last dose of mocetinostat.
15. Males with partner(s) of childbearing potential must take appropriate precautions to avoid fathering a child from the screening period until 90 days after receiving the last dose of mocetinostat. They must commit to abstinence from heterosexual intercourse or agree to use appropriate barrier contraception.
16. Prior to enrollment of females or males of reproductive potential, the investigator must document confirmation of the subject's understanding of the possible teratogenic effects of mocetinostat.
17. Willingness and ability to comply with scheduled visits, treatment plan, laboratory tests and other study procedures.

### Exclusion Criteria:

Subjects will not be enrolled if they meet any one of the following exclusion criteria:
1. Current participation in another therapeutic clinical trial.
2. Symptomatic brain metastases.
3. History of previous cancer (non RMS), except squamous cell or basal-cell carcinoma of the skin or any in situ carcinoma that has been completely resected, which required therapy within the previous 3 years. Other low grade cancers can be reviewed and allowed at the discretion of the PI.
4. Incomplete recovery from any surgery (other than central venous catheter or port placement) prior to treatment.
5. Any of the following in the past 6 months: pericarditis, pericardial effusion, symptomatic congestive heart failure, cerebrovascular accident or transient ischemic attack, pulmonary embolism, deep vein thrombosis, symptomatic bradycardia, requirement for anti-arrhythmic medication.
6. History of prolonged QTc interval (e.g., repeated demonstration of a QTc interval >450 milliseconds, unless associated with the use of medications known to prolong the QTc interval). QTc will be calculated using the Bazett formula (RR interval=60/heart rate; QTI Corrected=QT interval/sqr (RRinterval)).
7. History of additional risk factors for torsade de pointes (e.g., heart failure, family history of long QT syndrome).
8. Use of concomitant medications that increase or possibly increase the risk to prolong the QTc interval and/or induce torsades de pointes ventricular arrhythmia.
9. Females who are breastfeeding/lactating.
10. Known active infections (e.g., bacterial, fungal, viral including hepatitis and HIV positivity).
11. Other severe acute or chronic medical or psychiatric condition or laboratory abnormality that may increase the risk associated with study participation or study drug administration or may interfere with the interpretation of study results and, in the judgment of the Investigator, would make the Subject inappropriate for entry into this study or compromise protocol objectives in the opinion of the Investigator and/or the Sponsor.

### Test Drugs, Dosage, and Mode of Administration:

Mocetinostat is formulated as 20 mg and 50 mg hard gelatin capsules. The composition of the drug product consists of a blend of mocetinostat free base drug substance, microcrystalline cellulose (Avicel® PH112), sodium starch glycolate, colloidal silicon dioxide and magnesium stearate (non-bovine). The drug product must be stored in the original packaging under storage conditions on label instructions 20 and 50 mg capsules stored at room temperature conditions between 15° C. and 30° C.).

The appropriate dose of mocetinostat to be administered to each subject will be calculated at each treatment cycle.

The starting dose level for the Phase 1 dose escalation portion will be 40 mg. Dose will be increased until the MTD or the RP2D has been determined.

A treatment cycle will consist of mocetinostat administered orally three times per week starting on day 3 for a total of 9 doses per 21 day cycle (1 cycle=21 days). Vinorelbine will also be given, please see below. All subjects are eligible to receive repeated cycles as long as in the Principal Investigator's judgment, continued treatment is warranted.

Subjects should not be given G-CSF or other myeloid growth factors during Cycle 1, except to alleviate neutropenia in subjects with documented bacterial sepsis after initiating treatment with mocetinostat and vinorelbine or as defined in the dose-escalation plan.

Subjects experiencing ≥Grade 2 neutropenia during any cycle may receive G-CSF or other myeloid growth factors in subsequent cycles.

Other study drugs, dosage, mode of administration:

Vinorelbine is formulated as an injectable solution containing an equivalent of 10 mg (1 ml vial or 50 mg (5 ml vial) in sterile water. The drug product must be stored in the original packaging under storage conditions on label instructions. Unopened vials of vinorelbine are stable at temperatures up to 25° C. (77° F.) for up to 72 hours or until the date indicated on the package when stored under refrigeration at 2-8° C. (36-46° F.) and protected from light in the carton. Diluted vinorelbine may be used for up to 24 hours under normal room light when stored in polypropylene syringes or polyvinyl chloride bags at 5-30° C. (41-85° F.).

The vinorelbine dose will be 25 mg/m2/dose.

The dose of vinorelbine is appropriate dose of vinorelbine to be administered to each subject will be calculated at each treatment cycle based on the subject's BSA.

A treatment cycle will consist of vinorelbine administered intravenously via a central venous catheter on days 1, 8, and 15 of 21 day cycles. Mocetinostat will also be administered, please see above. All subjects are eligible to receive repeated cycles as long as in the Principal Investigator's judgment, continued treatment is warranted.

Subjects in cohorts 1, 2 and 3 and the expansion cohort will be seen in clinic for all 3 days of vinorelbine infusions during each cycle. Subjects should not be given G-CSF or other myeloid growth factors during Cycle 1, except to alleviate neutropenia in subjects with documented bacterial sepsis after initiating treatment with mocetinostat and vinorelbine or as defined in the dose-escalation plan.

Subjects experiencing ≥Grade 2 neutropenia during any cycle may receive G-CSF or other myeloid growth factors in subsequent cycles.

### Drug Administration:

Mocetinostat will be administered orally three times a week starting on day 3 for a total of 9 doses for each 21 day cycle.

Vinorelbine will be administered intravenously via a central venous catheter on days 1, 8 and 15 of each 21 day cycle.

### Duration of Treatment:

Treatment with mocetinostat and vinorelbine may continue until the subject experiences disease progression as defined in RECIST 1.1, withdraws consent, or experiences unacceptable toxicity. Treatment may continue as long as the Investigator believes the subject continues to derive clinical benefit in the absence of disease progression. There is no limit on the number of cycles a subject can receive.

### Criteria for Evaluation:

### Safety:

Safety and tolerability of mocetinostat as characterized by type, severity (graded using NCI CTCAE v4.03), timing, and relationship to study therapy of all adverse events and laboratory abnormalities, abnormal ECGs and physical exam findings in the first and in subsequent cycles. Laboratory values will be summarized by toxicity grade.

### Pharmacokinetics:

PK samples will be obtained from all subjects to determine the systemic exposure to mocetinostat. PK parameters such as Css, CL, Vd, and t1/2 will be calculated for each subject using non-compartmental methods. For the dose escalation segment, blood samples will be collected at the following times for all subjects in each cohort: For the first treatment cycle, subject will take their oral dose of mocetinostat on day 14 preferably in the morning to enable more convenient times for PK sampling. Blood samples for PK analysis will be drawn pre-dose, and at 1, 3 and 7 hours after mocetinostat dose.

### Pharmacodynamics:

Pharmacodynamic studies will be conducted on blood samples obtained from all subjects to determine potential biomarkers. Archived tumor samples may be requested at a future date for exploratory biomarkers and/or other analyses. Pharmacodynamic blood samples will be collected at the following times during Cycle 1: day 1 prior to vinorelbine dosing and day 14 pre-mocetinostat dosing and 3 hours post-mocetinostat dosing.

### Criteria for Evaluation:

### Phase 1 Dose Escalation

### Primary Endpoint:

First cycle DLTs, MTD, and RP2D DLTs will be graded according to the NCI CTCAE v4.03. DLT will be defined as any of the following events for which causal relationship to mocetinostat cannot be excluded.

| Category | Criteria |
|---|---|
| Hematology | Grade 4 neutropenia (absolute neutrophil count |
| toxicities | [ANC] <500/mm3) lasting >7 days; |
| | Grade 4 anemia; |
| | Febrile neutropenia (ANC <1000/mm3 with a single |
| | temperature of >38.3° C. or a sustained temperature |
| | of ≥38° C. for more than one hour); |
| | Grade ≥3 neutropenic infection (i.e., infection |
| | documented clinically or microbiologically with |
| | grade ≥3 neutropenia); |
| | Grade 4 thrombocytopenia (platelet count <25,000/mm3); |
| | Grade 3 thrombocytopenia (platelet count <50,000- |
| | 25,000/mm3) lasting >7 days or associated with |
| | clinically significant bleeding. |
| Gastrointestinal | All ≥Grade 4 vomiting or diarrhea; |
| toxicities | Grade 3 nausea or vomiting despite optimal antiemetic |
| | therapy that fails to recover to at least Grade 2 |
| | within 72 hours; |
| | Grade 3 diarrhea despite optimal management of the |
| | event that fails to recover to at least Grade 2 |
| | within 72 hours. |
| CNS toxicities | Grade ≥3 |
| Other | Grade ≥3; |
| nonhemato- | Grade 2 increase in AST/ALT in combination with a |
| logical | grade 2 increase in bilirubin; |
| toxicities | For subjects with liver metastases with elevated |
| | liver transaminases at baseline (2.5-5x ULN), |
| | DLT shall be defined as a doubling of the baseline |
| | liver transaminase value(s); |
| Failure to | Failure to recover to Grade ≤2 toxicity or to |
| recover (except | baseline values after delaying the initiation of |
| alopecia) | next cycle for a maximum of 21 days |

To determine PFS of the RP2D of mocetinostat administered orally three times per week starting on day 3 for a total of 9 doses a 21 day cycle, given in combination with vinorelbine on days 1, 8, 15 of a 21 day cycle in subjects with refractory or recurrent RMS. PFS is defined as time from first dose of vinorelbine to tumor progression or death due to any cause.

### Secondary Endpoints:

Determine PFS at 4 months and 6 months.
ORR is defined as the proportion of subjects with a confirmed CR or PR according to RECIST v1.1 as assessed by the Investigator.
DC according to RECIST v1.1 defined as the proportion of subjects with a confirmed CR, PR, or SD
DOR as defined from the first date a response is identified (either CR or PR) until the date of disease
Pharmacodynamic/plasma inhibitory profile of mocetinostat against molecular targets of interest to help confirm the biologically effective dose and RP2D of mocetinostat.

### Statistical Methods:

The clinical outcomes, laboratory, PK, and other safety data from both segments of the study will be analyzed descriptively. In addition to determining DLT and RP2D, results will be analyzed to determine if a sufficient response signal and safety profile justifies further study. Descriptive statistical summaries for demographic and subject baseline characteristics will be produced, as well as statistical summaries of safety, efficacy and pharmacokinetic/pharmacodynamic results, where categories for statistical summaries will consist of the dose level initially assigned for Phase 1 dose escalation, and the recommended initial dose in Phase 2 (RP2D). In addition, exploratory analyses of both toxicity, response (ORR) and pharmacodynamic data will be performed for both the assigned and actual daily dose of drug, the actual number of days of treatment, and for cumulative exposure to study drug as expressed by the product sum of dose over time (area under the dose-time curve).

### Safety:

All subjects who receive any amount of study drug will be included in the safety analyses. All adverse events will be mapped to preferred terms and system organ classes using CTCAE v5.0. Subject incidence of adverse events will be displayed by dose group and by system organ class. Adverse events will also be summarized by severity and relationship to study drug. Subject incidence of serious adverse events will also be summarized. The type and number of DLTs will be separately presented by dose group, as appropriate. Laboratory parameters will be summarized using descriptive statistics at baseline and at each post-baseline time point. Changes from baseline will also be summarized.

### Pharmacokinetics:

Pharmacokinetic parameter values will be summarized by descriptive statistics at each dose level.

### Pharmacodynamics:

Pharmacodynamic variables will be summarized by dose group and time point. Correlations between pharmacodynamic variables and efficacy variables may also be performed.

### Efficacy:

A modified intent to treat (mITT) approach will be used for efficacy analysis, in which the mITT population will consist of all subjects who receive any amount of study drug. Tumor response rates will be summarized by dose group and for all subjects who receive the RP2D, including those from the dose escalation and expansion phases. Responses will be classified as CR, PR, SD or PD according to RECIST v1.1 criteria. Summaries will be based on the best response recorded up until disease progression. Subjects who discontinue prior to the first 6-weekly response assessment will be considered as non-responders in the primary efficacy analysis. Objective tumor response (CR or PR) will also be summarized, as will PFS, OS, DCR and duration of response and DCR. Time to event data will be summarized by Kaplan-Meier methods, including 25th, 50th (median) and 75th percentiles with point estimates and two-sided 95% confidence intervals, as well as number and percent of censored observations.

### Sample Size:

The sample size for the dose-escalation phase of the study will be determined by the required sample within each cohort (3 or 6 subjects). The sample size for the expansion phase will be 20 subjects.

### Schedules

Schedule of Medication administration
and study visits for all cycles

| | | Vinorelbine | Mocetinostat | |
|---|---|---|---|---|
| Week | Day | dosing | dosing | Study Visit |
| 1 | 1 | Vinorelbine | | * |
| 1 | 2 | | | |
| 1 | 3 | | Mocetinostat | |
| 1 | 4 | | | |
| 1 | 5 | | Mocetinostat | |
| 1 | 6 | | | |
| 1 | 7 | | Mocetinostat | |
| 2 | 8 | Vinorelbine | | * |
| 2 | 9 | | | |
| 2 | 10 | | Mocetinostat | |
| 2 | 11 | | | |
| 2 | 12 | | Mocetinostat | |
| 2 | 13 | | | |
| 2 | 14 | | Mocetinostat | * (PK collection) |
| 3 | 15 | Vinorelbine | | * |
| 3 | 16 | | | |
| 3 | 17 | | Mocetinostat | |
| 3 | 18 | | | |
| 3 | 19 | | Mocetinostat | |
| 3 | 20 | | | |
| 3 | 21 | | Mocetinostat | |

Schedule of Events (Phase 1 Dose Escalation Cohort)
Screening, Cycle 1

| | | | | | | | Early |
|---|---|---|---|---|---|---|---|
| Assessment | Screening | Day 1 | Day 8 | Day 14 | Day 15 | Day 22ⁿ | Term/EOT^{o} |
| Informed Consent^{a} | X | | | | | | |
| Demographics | X | | | | | | |
| Medical History | X | X | | | | X | |
| ECOG or | X | X | | | | X | X |
| Karnofsky/Lansky | | | | | | | |
| Performance Scale^{b}** | | | | | | | |
| Outpatient | X | X | X | | X | X | X |
| clinic visit | | | | | | | |
| Adverse events | | X | X | | X | X | X |
| Concomitant | X | X | X | | X | X | X |
| Medications^{c} | | | | | | | |
| Urine pregnancy | X | X | | | | X | X |
| test (females)** | | | | | | | |
| Height^{d}** | X | X | | | | X | X |
| Weight^{d}** | X | X | X | | X | X | X |
| Complete | X | X | | | | X | X |
| Physical Exam | | | | | | | |
| Symptom-drive | | | X | | X | | |
| physical exam^{e} | | | | | | | |
| Vital signs^{f} | X | X | X | | X | X | X |
| 12-lead ECG^{g} | X | | | | | X | X |
| ECHO^{h} | X | | | | | X | |
| Hematology, | X | X | X | | X | X | X |
| Clinical | | | | | | | |
| Chemistryⁱ** | | | | | | | |
| Urinalysisⁱ** | X | X | | | | X | X |
| Vinorelbine | | X | X | | X | X | |
| administration | | | | | | | |
| Mocetinostat | | | | X | | | |
| administration^{j} | | | | (in | | | |
| | | | | clinic) | | | |
| Pharmacokinetic | | | | X | | | |
| samples^{k} | | | | | | | |
| Pharmacodynamic | | X | | X | | | X |
| samples^{l} | | | | | | | |
| Tumor/Disease | X | | | | | | X |
| Evaluation^{m} | | | | | | | |

Schedule of Events (Phase 1 Dose Escalation Cohorts; Screening, Cycle 1)
^{a}For subjects <18 years of age, their parent or legal guardian must sign a written informed consent. Assent, when appropriate, will be obtained according to institutional guidelines.
^{b}ECOG performance status in subjects ≥17 years old; Karnofsky/Lansky status in subjects ≤16 years old.
^{c}Screening: Chronic medications; Baseline; medications taken <14 days prior to first dose of study medication; Post dosing: Concomitant medications.
^{d}Screening and day 1: weight and height; weight thereafter.
^{e}As needed, determined by the Principal Investigator’s interpretation of available safety data and subject-reported adverse events.
^{f}Temperature, heart rate, respiratory rate and blood pressure will be measured in the seated position, after at least 12 minutes rest.
^{g}A 12-lead ECG will be done at screening.
^{h}An ECHO will be done at screening and within 1 week prior to starting cycles 1,2,3 and4, and at the end of the study. If the screening ECHO has been done within 1 week prior to cycle 1, day 1, it does not need to be repeated in order to start cycle 1, day 1.
ⁱHematology, clinical chemistry, and urinalysis will be performed at Screening and on Day 1. Hematology and clinical chemistry will be performed on Day 8 and Day15.
i. hematology: complete blood count (CBC) including WBC with differential (neutrophils, lymphocytes, monocytes, eosinophils, basophils) RBC, hemoglobin, hematocrit, and platelet count;
ii. clinical chemestry: serum sodium, potassium, chloride, bicarbonate, BUN, creatinine, eGFR, calcium, gluclose, total bilirubin (direct (conjugated)/indirect (unconjugated) bilirubin fractionation is only needed if total bilirubin is abnormal), alanine transaminase (ALT), aspartate transaminase (AST), alkaline phosphate, total protein, albumin;
iii. urinalysis: color, protein, glucose, bilirubin, ketones, blood, pH, specific gravity, leukocytes, and leukocyte esterase (NOTE: urinalysis is not required at the Day 8 and day 15 visits).
^{j}Mocetinostat will be administered during the clinic visit on cycle 1, day 14. Other doses of mocetinostat will be given by the subject, three times per week starting on day 3 for a total of 9 doses per 21 day cycle (Days 3, 5, 7, 10, 12, 14, 17, 19, 21). Administration is PO.
^{k}To facilitate collection of pharmacokinetic samples, mocetinostat should be administered in the morning on Day 14. Plasma PK samples will be collected pre-dose on Day 14, and 1, 3, and 7 hours post-dose.
^{l}Pharmacodynamic samples will be collected on day 1 prior to vinorelbine administration and on day 14 pre- moncetinostat dosing and 3 hours post-moncetinostat dosing
^{m}Tumor assesments will be done by CT scan or MRI q6weeks +/− 1 week. The same method should be used throughout the study.
ⁿIf the subject is eligible to continue to Cycle 2, the Day 22 visit can be considered the Day 1 visit of the next cycle. If a subject completes Cycle 1 and does not continue, the subject will be considered to have completed the study. Any subject discontinuing after completing Cycle 1 should complete the early termination visit rather than the Day 22 visit (but will indicate in the CRF that the subject has completed the study). Subsequent cycles will begin not earlier than 21 days, and not later than 3 days from the scheduled start date
^{o}The early termination visit will be done if the subject discontinues prior to the end of the cycle and should be done within 2 days of discontinuation. The End of Treatment visit (EOT) will be conducted if the subject completes a cycle and doesn’t continue treatment. The EOT visit should be done either on the last day of the cycle (Day 22) or within 7 days after the completion of the cycle. A tumor assessment should be done if the subject terminates at a time that coincides with when a tumor assessment would normally be done.
**If the screening assessments for ECOG or Karnofsky/Lansky performance status, height, weight, clinical laboratory tests and urine pregnancy test were performed within approximately 72 hours prior to dosing on Day 1 of Cycle 1, these assessments do not need to be repeated except as required by institutional standards.

Schedule of Events (Phase 1 Dose Escalation Cohort)
Cycles 2, 3, 4

| | | | | | Early |
|---|---|---|---|---|---|
| Assessment | Day 1 | Day 8 | Day 15 | Day 22J | Term/EOT^{k} |
| Demographics | | | | | |
| Medical History | X | | | X | |
| ECOG or | X | | | X | X |
| Karnofsky/Lansky | | | | | |
| Performance Scale^{a} | | | | | |
| Outpatient clinic | X | X | X | X | X |
| visit | | | | | |
| Adverse events | X | X | X | X | X |
| Concomitant | X | X | X | X | X |
| Medications^{b} | | | | | |
| Urine pregnancy test | X | | | X | X |
| (females) | | | | | |
| Height^{c} | X | | | X | X |
| Weight^{c} | X | X | X | X | X |
| Complete Physical | X | | | X | X |
| Exam | | | | | |
| Symptom-drive | | X | X | | |
| physical exam^{d} | | | | | |
| Vital signs^{e} | X | X | X | X | X |
| 12-lead ECG | X | | | X | X |
| ECHO^{f} | X | | | X | X |
| Hematology, | X | X | X | X | X |
| Clinical | | | | | |
| Chemistry^{g} | | | | | |
| Urinalysis^{g} | X | | | X | X |
| Vinorelbine | X | X | X | X | |
| administration | | | | | |
| Mocetinostat | | | | | |
| administration^{h} | | | | | |
| Pharmacodynamic | | | | | X |
| samples | | | | | |
| Tumor/Disease | | | | | X |
| Evaluationⁱ | | | | | |

Schedule of Events Footnotes (Phase 1 Dose Escalation Cohorts; Cycle 2, 3, 4)
^{a}ECOG performance status in subjects ≥17 years old; Karnofsky/Lansky status in subjects ≤16 years old.
^{b}Screening: Chronic medications; Baseline: Medications taken <14 days prior to first dose of study medication; Post dosing: Concomitant medications.
^{c}Day 1: weight and height; weight thereafter.
^{d}As needed, determined by the Principal Investigator's interpretation of available safety data and subject-reported adverse events.
^{e}Temperature, heart rate, respiratory rate and blood pressure will be measured in the seated position, after at least 2 minutes rest.
^{f}An ECHO will be done at screening and within 1 week prior to starting cycles 1, 2, 3 and 4, and at the end of the study. If the screening ECHO has been done within 1 week prior to cycle 1, day 1, it does not need to be repeated in order to start cycle 1, day 1.
^{g}Hematology, clinical chemistry, and urinalysis will be preformned at Screening and on Day 1. Hematology and clinical chemistry will be preformed on Day 8 and Day 15.
i. hematology: complete blood count (CBC) including WBC with differential (neutrophils, lymphocytes, monocytes, eosinophils, basophils) RBC, hemoglobin, hematocrit, and platelet count;
ii. clinical chemistry: serum sodium, potassium, chloride, bicarbonate, BUN, creatinine, eGFR, calcium, glucose, toal bilirubin (direct (conjugated)/indirect (unconjugated) bilirubin fractionation is only needed if total bilirubin is abnormal), alanine transaminase (ALT), aspartate transaminase (AST), alkaline phosphatase, total protein, albumin;
iii. urinalysis: color, protein, glucose, bilirubin, ketones, blood, pH, specific gravity, leukocytes and leukocyte esterase
(NOTE:
urinalysis is not required at the Day 8 and day 15 visits).
^{h}Mocetinostat will be administered by the subject, three times per week starting on day 3 for a toal of 9 doses per 21 day cycle (Days 3, 5, 7, 10, 12, 14, 17, 19, 21). Administration is PO.
ⁱTumor assessments will be done by CT scan or MRI q6 weeks +/− 1week. The same method should be used throughout the study.
^{j}If the subject is eligible to continue to Cycles 2, 3, and 4 the Day 22 visit can be considered the Day 1 visit of the next cycle. If a subject completes a cycle and does not continue, the subject will be considered to have completed the study. Any subject discontinuing after completing of a cycle should complete the early termination visit rather than the Day 22 visit (but will indicate in the CRF that the subject has completed the study). Subsequent cycles will begin not earlier than 21 says, and not later than 3 days from the scheduled start date.
^{k}The early termination visit will be done if the subject discontinues prior to the end of the cycle and should be done within 2 days of disontinuation. The EOT visit should be done either the last day of the cycle (Day 22) or within 7 days after the completion of the cycle. A tumor assessment should be done if the subject terminates at a time that coincides with when a tumor assessment would normally be done.

Schedule of Events (Phase 1 Dose Escalation
Cohort) Cycle 5 and subsequent cycles

| | | | | | Early |
|---|---|---|---|---|---|
| Assessment | Day 1 | Day 8 | Day 15 | Day 22J | Term/EOT^{k} |
| Demographics | | | | | |
| Medical History | X | | | X | |
| ECOG or | X | | | X | X |
| Karnofsky/Lansky | | | | | |
| Performance Scale^{a} | | | | | |
| Outpatient clinic | X | X | X | X | X |
| visit | | | | | |
| Adverse events | X | X | X | X | X |
| Concomitant | X | X | X | X | X |
| Medications^{b} | | | | | |
| Urine pregnancy test | X | | | X | X |
| (females) | | | | | |
| Height^{c} | X | | | X | X |
| Weight^{c} | X | X | X | X | X |
| Complete Physical | X | | | X | X |
| Exam | | | | | |
| Symptom-drive | | X | X | | |
| physical exam^{d} | | | | | |
| Vital signs^{e} | X | X | X | X | X |
| 12-lead ECG | X | | | X | X |
| ECHO^{f} | | | | | X |
| Hematology, | X | X | X | X | X |
| Clinical | | | | | |
| Chemistry^{g} | | | | | |
| Urinalysis^{g} | X | | | X | X |
| Vinorelbine | X | X | X | X | |
| administration | | | | | |
| Mocetinostat | | | | | |
| administration^{h} | | | | | |
| Pharmacodynamic | | | | | X |
| samples | | | | | |
| Tumor/Disease | | | | | X |
| Evaluationⁱ | | | | | |

### Schedule of Events Footnotes (Phase 1 Dose Escalation Cohorts; Cycle 5 and Subsequent Cycles)

a) ECOG performance status in subjects ≥17 years old; Karnofsky/Lansky status in subjects ≤16 years old.
b) Screening: Chronic medications; Baseline: Medications taken <14 days prior to first dose of study medication; Post dosing: Concomitant medications.
c) Day 1: weight and height; weight thereafter.
d) As needed, determined by the Principal Investigator's interpretation of available safety data and subject-reported adverse events.
e) Temperature, heart rate, respiratory rate and blood pressure will be measured in the seated position, after at least 2 minutes rest.
f) An ECHO will be done at screening and within 1 week prior to starting
f) cycles 1, 2, 3 and 4, and at the end of the study. If the screening ECHO has been done within 1 week prior to cycle 1, day 1, it does not need to be repeated in order to start cycle 1, day 1.
g) Hematology, clinical chemistry, and urinalysis will be performed at Screening and on Day 1. Hematology and clinical chemistry will be performed on Day 8 and Day 15.
h) hematology: complete blood count (CBC) including WBC with differential (neutrophils, lymphocytes, monocytes, eosinophils, basophils) RBC, hemoglobin, hematocrit, and platelet count;
   i. clinical chemistry: serum sodium, potassium, chloride, bicarbonate, blood urea nitrogen, creatinine, calcium, glucose, total bilirubin (direct (conjugated)/indirect
   ii. (unconjugated) bilirubin fractionation is only needed if total bilirubin is abnormal), alanine transaminase (ALT), aspartate transaminase (AST), alkaline phosphatase, total protein, albumin;
   iii. urinalysis: color, protein, glucose, bilirubin, ketones, blood, pH, specific gravity, leukocytes, and leukocyte esterase (NOTE: urinalysis is not required at the Day 8 and day 15 visits).
i) Mocetinostat will be administered by the subject, three times per week starting on day 3 for a total of 9 doses per 21 day cycle (Days 3, 5, 7, 10, 12, 14, 17, 19, 21). Administration is PO.
j) Tumor assessments will be done by CT scan or MRI q6 weeks+/−1 week. The same method should be used throughout the study.
k) If the subject is eligible to continue to the next cycle, the Day 22 visit can be considered the Day 1 visit of the next cycle. If a subject completes a cycle and does not continue, the subject will be considered to have completed the study. Any subject discontinuing after completing a cycle should complete the early termination visit rather than the Day 22 visit (but will indicate in the CRF that the subject has completed the study). Subsequent cycles will begin not earlier than 22 days, and not later than 3 days from the scheduled start date
l) The early termination visit will be done if the subject discontinues prior to the end of the cycle and should be done within 2 days of discontinuation. The End of Treatment visit (EOT) will be conducted if the subject completes a cycle and doesn't continue treatment. The EOT visit should be done either on the last day of the cycle (Day 22) or within 7 days after the completion of the cycle. A tumor assessment should be done if the subject terminates at a time that coincides with when a tumor assessment would normally be done.

Schedule of Events (Phase 2 Dose Expansion cohort)
Screening, Cycle 1

| | | | | | | | Early |
|---|---|---|---|---|---|---|---|
| Assessment | Screening | Day 1 | Day 8 | Day 14 | Day 15 | Day 22^{m} | Term/EOTⁿ |
| Informed Consent^{a} | X | | | | | | |
| Demographics | X | | | | | | |
| Medical History | X | X | | | | X | |
| ECOG or | X | X | | | | X | X |
| Karnofsky/Lansky | | | | | | | |
| Performance Scale^{b}** | | | | | | | |
| Outpatient clinic | X | X | X | | X | X | X |
| visit | | | | | | | |
| Adverse events | | X | X | | X | X | X |
| Concomitant | X | X | X | | X | X | X |
| Medications^{c} | | | | | | | |
| Urine pregnancy | X | X | | | | X | X |
| test (females)** | | | | | | | |
| Height^{d}** | X | X | | | | X | X |
| Weight^{d}** | X | X | X | | X | X | X |
| Complete Physical | X | X | | | | X | X |
| Exam | | | | | | | |
| Symptom-drive | | | X | | X | | |
| physical exam^{e} | | | | | | | |
| Vital signs^{f} | X | X | X | | X | X | X |
| 12-lead ECG | X | | | | | X | X |
| ECHO^{g} | X | | | | | X | X |
| Hematology, | X | X | X | | X | X | X |
| Clinical | | | | | | | |
| Chemistry^{h}** | | | | | | | |
| Urinalysis^{h}** | X | X | | | | X | X |
| Vinorelbine | | X | X | | X | X | |
| administration | | | | | | | |
| Mocetinostat | | | X | X | | | |
| administrationⁱ | | | (in | (in | | | |
| | | | clinic) | clinic) | | | |
| Pharmacokinetic | | | X | X | | | |
| samples^{j} | | | | | | | |
| Pharmacodynamic | | X | | X | | | X |
| samples^{k} | | | | | | | |
| Tumor/Disease | X | | | | | | X |
| Evaluation^{l} | | | | | | | |

### Schedule of Events Footnotes (Phase 2 Dose Expansion Cohort; Screening, Cycle 1)

a) For subjects <18 years of age, their parent or legal guardian must sign a written informed consent. Assent, when appropriate, will be obtained according to institutional guidelines.
b) ECOG performance status in subjects ≥17 years old; Karnofsky/Lansky status in subjects ≤16 years old.
c) Screening: Chronic medications; Baseline: Medications taken <14 days prior to first dose of study medication; Post dosing: Concomitant medications.
d) Screening and day 1: weight and height; weight thereafter.
e) As needed, determined by the Principal Investigator's interpretation of available safety data and subject-reported adverse events.
f) Temperature, heart rate, respiratory rate and blood pressure will be measured in the seated position, after at least 2 minutes rest.
g) An ECHO will be done at screening and within 1 week prior to starting cycles 1, 2, 3 and 4, and at the end of the study. If the screening ECHO has been done within 1 week prior to cycle 1, day 1, it does not need to be repeated in order to start cycle 1, day 1.
h) Hematology, clinical chemistry, coagulation and urinalysis will be performed at Screening and on Day 1. Hematology and clinical chemistry will be performed on Day 8 and Day 15.
   i. hematology: complete blood count (CBC) including WBC with differential (neutrophils, lymphocytes, monocytes, cosinophils, basophils) RBC, hemoglobin, hematocrit, and platelet count;
   ii. clinical chemistry: serum sodium, potassium, chloride, bicarbonate, BUN, creatinine, calcium, glucose, eGFR, total bilirubin (direct (conjugated)/indirect (unconjugated) bilirubin fractionation is only needed if total bilirubin is abnormal), alanine transaminase (ALT), aspartate transaminase (AST), alkaline phosphatase, lactate dehydrogenase (LDH), total protein, albumin;
   iii. urinalysis: color, protein, glucose, bilirubin, ketones, blood, pH, specific gravity, leukocytes and leukocyte esterase (NOTE: urinalysis is not required at the Day 8 and day 15 visits).
i) Mocetinostat will be administered during the clinic visit on cycle 1, day 14. Other doses of mocetinostat will be given by the subject, three times per week starting on day 3 for a total of 9 doses per 21 day cycle (Days 3, 5, 7, 10, 12, 14, 17, 19, 21). Administration is PO.
j) To facilitate collection of pharmacokinetic samples, mocetinostat should be administered in the morning on Day 14. Plasma PK samples will be collected pre-dose on Day 1, and 1, 3 and 7 hours post-dose.
k) Pharmacodynamic samples will be collected on day 1 prior to vinorelbine administration and on day 14 pre-mocetinostat dosing and 3 hours post-mocetinostat dosing
l) Tumor assessments will be done by CT scan or MRI q6 weeks+/−1 week. The same method should be used throughout the study.
m) If the subject is eligible to continue to Cycle 2, the Day 22 visit can be considered the Day 1 visit of the next cycle. If a subject completes Cycle 1 and does not continue, the subject will be considered to have completed the study. Any subject discontinuing after completing Cycle 1 should complete the early termination visit rather than the Day 22 visit (but will indicate in the CRF that the subject has completed the study). Subsequent cycles will begin not earlier than 21 days, and not later than 3 days from the scheduled start date
n) The early termination visit will be done if the subject discontinues prior to the end of the cycle and should be done within 2 days of discontinuation. The End of Treatment visit (EOT) will be conducted if the subject completes a cycle and doesn't continue treatment. The EOT visit should be done either on the last day of the cycle (Day 22) or within 7 days after the completion of the cycle. A tumor assessment should be done if the subject terminates at a time that coincides with when a tumor assessment would normally be done.
**If the screening assessments for ECOG or Karnofsky/Lansky performance status, height, weight, clinical laboratory tests and urine pregnancy test were performed within approximately 72 hours prior to dosing on Day 1 of Cycle 1, these assessments do not need to be repeated except as required by institutional standards.

Schedule of Events (Phase 2 Dose Expansion cohort)
Cycles 2, 3, 4

| | | | | | Early |
|---|---|---|---|---|---|
| Assessment | Day 1 | Day 8 | Day 15 | Day 22J | Term/EOT^{k} |
| Demographics | | | | | |
| Medical History | X | | | X | |
| ECOG or | X | | | X | X |
| Karnofsky/Lansky | | | | | |
| Performance Scale^{a} | | | | | |
| Outpatient clinic | X | X | X | X | X |
| visit | | | | | |
| Adverse events | X | X | X | X | X |
| Concomitant | X | X | X | X | X |
| Medications^{b} | | | | | |
| Urine pregnancy test | X | | | X | X |
| (females) | | | | | |
| Height^{c} | X | | | X | X |
| Weight^{c} | X | X | X | X | X |
| Complete Physical | X | | | X | X |
| Exam | | | | | |
| Symptom-drive | | X | X | | |
| physical exam^{d} | | | | | |
| Vital signs^{e} | X | X | X | X | X |
| 12-lead ECG | X | | | X | X |
| ECHO^{f} | X | | | X | X |
| Hematology, | X | X | X | X | X |
| Clinical | | | | | |
| Chemistry^{g} | | | | | |
| Urinalysis^{g} | X | | | X | X |
| Vinorelbine | X | X | X | X | |
| administration | | | | | |
| Mocetinostat | | | | | |
| administration^{h} | | | | | |
| Pharmacodynamic | | | | | X |
| samples | | | | | |
| Tumor/Disease | | | | | X |
| Evaluationⁱ | | | | | |

### Schedule of Events Footnotes (Phase 2 Dose Expansion Cohort; Cycles 2, 3, 4)

a) ECOG performance status in subjects ≥17 years old; Karnofsky/Lansky status in subjects ≤16 years old.
b) Screening: Chronic medications; Baseline: Medications taken <14 days prior to first dose of study medication; Post dosing: Concomitant medications.
c) Day 1: weight and height; weight thereafter.
d) As needed, determined by the Principal Investigator's interpretation of available safety data and subject-reported adverse events.
e) Temperature, heart rate, respiratory rate and blood pressure will be measured in the seated position, after at least 2 minutes rest.
f) An ECHO will be done at screening which must be within 1 week prior to starting cycle 1, within 1 week prior to starting cycle 2, cycle 3 and cycle 4 and at the end of the study.
g) Hematology, clinical chemistry, coagulation and urinalysis will be performed at Screening and on Day 1. Hematology and clinical chemistry will be performed on Day 8 and Day 15.
   i. hematology: complete blood count (CBC) including WBC with differential (neutrophils, lymphocytes, monocytes, cosinophils, basophils) RBC, hemoglobin, hematocrit, and platelet count;
   ii. clinical chemistry: serum sodium, potassium, chloride, bicarbonate, BUN, creatinine, calcium, glucose, eGFR, total bilirubin (direct (conjugated)/indirect (unconjugated) bilirubin fractionation is only needed if total bilirubin is abnormal), alanine transaminase (ALT), aspartate transaminase (AST), alkaline phosphatase, lactate dehydrogenase (LDH), total protein, albumin;
   iii. urinalysis: color, protein, glucose, bilirubin, ketones, blood, pH, specific gravity, leukocytes, and leukocyte esterase (NOTE: urinalysis is not required at the Day 8 and day 15 visits).
h) Mocetinostat will be administered by the subject, three times per week starting on day 3 for a total of 9 doses per 21 day cycle (Days 3, 5, 7, 10, 12, 14, 17, 19, 21). Administration is PO.
i) Tumor assessments will be done by CT scan or MRI q6 weeks+/−1 week. The same method should be used throughout the study.
j) If the subject is eligible to continue to Cycle 2, the Day 22 visit can be considered the Day 1 visit of the next cycle. If a subject completes a cycle and does not continue, the subject will be considered to have completed the study. Any subject discontinuing after completing a cycle should complete the early termination visit rather than the Day 22 visit (but will indicate in the CRF that the subject has completed the study). Subsequent cycles will begin not earlier than 21 days, and not later than 3 days from the scheduled start date
k) The early termination visit will be done if the subject discontinues prior to the end of the cycle and should be done within 2 days of discontinuation. The End of Treatment visit (EOT) will be conducted if the subject completes a cycle and doesn't continue treatment. The EOT visit should be done either on the last day of the cycle (Day 22) or within 7 days after the completion of the cycle. A tumor assessment should be done if the subject terminates at a time that coincides with when a tumor assessment would normally be done.

Schedule of Events (Phase 2 Dose Expansion cohort)
Cycle 5 and subsequent cycles

| | | | | | Early |
|---|---|---|---|---|---|
| Assessment | Day 1 | Day 8 | Day 15 | Day 29J | Term/EOT^{k} |
| Demographics | | | | | |
| Medical History | X | | | X | |
| ECOG or | X | | | X | X |
| Karnofsky/Lansky | | | | | |
| Performance Scale^{a} | | | | | |
| Outpatient clinic | X | X | X | X | X |
| visit | | | | | |
| Adverse events | X | X | X | X | X |
| Concomitant | X | X | X | X | X |
| Medications^{b} | | | | | |
| Urine pregnancy test | X | | | X | X |
| (females) | | | | | |
| Height^{c} | X | | | X | X |
| Weight^{c} | X | X | X | X | X |
| Complete Physical | X | | | X | X |
| Exam | | | | | |
| Symptom-drive | | X | X | | |
| physical exam^{d} | | | | | |
| Vital signs^{e} | X | X | X | X | X |
| 12-lead ECG | X | | | X | X |
| ECHO^{f} | X | | | X | X |
| Hematology, | X | X | X | X | X |
| Clinical | | | | | |
| Chemistry^{g} | | | | | |
| Urinalysis^{g} | X | | | X | X |
| Vinorelbine | X | X | X | X | |
| administration | | | | | |
| Mocetinostat | | X | | | |
| administration^{h} | | | | | |
| Pharmacodynamic | | | | | X |
| samples | | | | | |
| Tumor/Disease | | | | | X |
| Evaluationⁱ | | | | | |

### Schedule of Events Footnotes (Phase 2 Dose Expansion Cohort; Cycle 5 and Subsequent Cycles)

a) ECOG performance status in subjects ≥17 years old; Karnofsky/Lansky status in subjects ≤16 years old.
b) Screening: Chronic medications; Baseline: Medications taken <14 days prior to first dose of study medication; Post dosing: Concomitant medications.
c) Screening: weight and height; weight thereafter
d) As needed, determined by the Principal Investigator's interpretation of available safety data and subject-reported adverse events.
e) Temperature, heart rate, respiratory rate and blood pressure will be measured in the seated position, after at least 2 minutes rest.
f) An ECHO will be done at screening and within 1 week prior to starting cycles 1, 2, 3 and 4, and at the end of the study. If the screening ECHO has been done within 1 week prior to cycle 1, day 1, it does not need to be repeated in order to start cycle 1, day 1.
g) Hematology, clinical chemistry, coagulation and urinalysis will be performed at Screening and on Day 1. Hematology and clinical chemistry will be performed on Day 8 and Day 15.
   i. hematology: complete blood count (CBC) including WBC with differential (neutrophils, lymphocytes, monocytes, cosinophils, basophils) RBC, hemoglobin, hematocrit, and platelet count;
   ii. clinical chemistry: serum sodium, potassium, chloride, bicarbonate, BUN, creatinine, calcium, glucose, eGFR, total bilirubin (direct (conjugated)/indirect (unconjugated) bilirubin fractionation is only needed if total bilirubin is abnormal), alanine transaminase (ALT), aspartate transaminase (AST), alkaline phosphatase, lactate dehydrogenase (LDH), total protein, albumin;
   iii. urinalysis: color, protein, glucose, bilirubin, ketones, blood, pH, specific gravity, leukocytes, and leukocyte esterase (NOTE: urinalysis is not required at the Day 8 and day 15 visits).
h) Mocetinostat will be administered by the subject, three times per week starting on day 3 for a total of 9 doses per 21 day cycle (Days 3, 5, 7, 10, 12, 14, 17, 19, 21). Administration is PO.
i) Tumor assessments will be done by CT scan or MRI q6 weeks+/−1 week. The same method should be used throughout the study.
j) If the subject is eligible to continue to Cycle 2, the Day 22 visit can be considered the Day 1 visit of the next cycle. If a subject completes a cycle and does not continue, the subject will be considered to have completed the study. Any subject discontinuing after completing a cycle should complete the early termination visit rather than the Day 22 visit (but will indicate in the CRF that the subject has completed the study). Subsequent cycles will begin not earlier than 21 days, and not later than 3 days from the scheduled start date
k) The early termination visit will be done if the subject discontinues prior to the end of the cycle and should be done within 2 days of discontinuation. The End of Treatment visit (EOT) will be conducted if the subject completes a cycle and doesn't continue treatment. The EOT visit should be done either on the last day of the cycle (Day 29) or within 7 days after the completion of the cycle. A tumor assessment should be done if the subject terminates at a time that coincides with when a tumor assessment would normally be done.

Rationale. Rhabdomyosarcoma (RMS) is a rare form of soft tissue cancer that affects children, adolescents and adults. It is the most common soft tissue cancer in children. Greater than one half of cases are diagnosed in children younger than ten years of age (Ognjanovic et al., 2009) but it can occur at any age. Despite advances in the treatment for RMS, outcomes remain suboptimal. The 5-year survival for those with apparently localized disease is approximately 80% while for patients with metastatic disease it is approximately 30% (Punyko et al., 2005). Additionally, unfortunately, about 30% of patients with RMS will relapse. The prognosis for patient with recurrent RMS is very poor with 5-year survival after recurrence of approximately 20-50% (Pappo et al., 1999 and Winter et al., 2015). While RMS is rarer in adults, the outcomes are worse with overall five-year survival of 27%. (Sultan et al., 2009). Safe and effective treatments for those with refractory, metastatic, and progressive rhabdomyosarcoma are needed.

The first line treatment for RMS is based on risk-adapted protocols and is multimodal with conventional chemotherapy and surgery and/or radiation. Standard chemotherapy regimens consist of a combination of vincristine, dactinomycin with addition of cyclophosphamide and irinotecan for selected patients.

Patients who relapse or those with disease refractory to first-line treatment typically receive additional chemotherapy. Vinorelbine is a semi-synthetic vinca alkaloid that has shown efficacy as a single agent and part of multi-agent regimen with cyclophosphamide in refractory rhabdomyosarcoma (Casanova et al., 2002, Casanova et al, 2004, Kuttesch et al. 2009, Minard-Colin et al., 2012). Other regimens include doxorubicin, ifosfamide and etoposide; cyclophosphamide and topotecan, and others. The lack of highly effective treatment for patients with refractory, metastatic, or recurrent RMS has catalyzed research into new approaches for treatment.

HDACs are well recognized enzymes involved in cancer pathogenesis. HDACs result in silencing of gene expression and lead to cell proliferation, evasion of apoptosis tumor growth and metastasis. Mocetinostat is an investigational selective HDAC inhibitor that binds to and inhibits HDAC1, HDAC2, HDAC3, and HDAC11. This alters gene expression including increasing expression of tumor suppressor genes which may be essential to halting tumorigenesis. This combination therapy is a novel approach to the treatment of RMS. The current study will demonstrate the efficacy of mocetinostat in combination with vinorelbine for recurrent RMS or RMS refractory to first line therapies. As discussed above, vinorelbine is a drug which is already used for refractory RMS and human tumor xenograft data shown in FIG. 1 shows that mocetinostat enhances the efficacy of vinorelbine.

### Objectives

Phase 1 Dose Escalation The primary objective of the Phase 1 dose escalation segment is to determine the first cycle dose-limiting toxicities (DLTs), maximum tolerated dose (MTD), and a biologically effective and recommended Phase 2 dose (RP2D) of Mocetinostat administered orally three times per week for a total of 9 doses per 21 day cycle given in combination with vinorelbine on days 1, 8, 15 of 21 day cycles in subjects with refractory or recurrent RMS.

Secondary objectives of the dose escalation segment of the study are to assess:
Safety profile of Mocetinostat in combination with vinorelbine as characterized by Adverse Event (AE) type, severity, timing and relationship to study drugs, as well as laboratory abnormalities in the first and subsequent treatment cycles
Pharmacokinetics (PK) of Mocetinostat in plasma
Clinical benefit rate (CBR=complete response (CR)+partial response (PR) and stable disease (SD)) of mocetinostat+vinorelbine in metastatic/refractory/unresectable RMS
Antitumor activity of mocetinostat+vinorelbine in refractory/recurrent RMS as measured by Overall Response Rate (ORR), Duration of Response (DOR), Disease Control (DC), Duration of Disease Control, as well as Progression-Free Survival (PFS)
   ORR includes Complete Response (CR) and Partial Response (PR)
   DOR as defined from the first date a response is identified (either CR or PR) until the date of disease progression
   Disease Control (DC) defined as the proportion of subjects with a confirmed CR, PR or Stable Disease (SD)
   Duration of Disease Control defined as first date of disease control identified (either CR, PR or SD) until the date of progression
   PFS as defined by time from first dose of vinorelbine to tumor progression or death due to any cause
Pharmacodynamics of mocetinostat on molecular targets in surrogate tissue
Exploratory biomarker development to enable prediction of drug toxicity, tumor response and the mechanism(s) of acquired study drug resistance.
Obtain RMS tissue biological samples pre-treatment and at progression to assess for differences in gene expression by Next Gen Sequencing and RNA Seq.

Expansion Cohort. The primary objective of the expansion cohort is to determine the PFS, defined as time from first dose of vinorelbine to tumor progression or death due to any cause, at the RP2D of mocetinostat administered orally three times per week starting on day 3 for a total of 9 doses per 21 day cycle given in combination with vinorelbine on days 1, 8, 15 of a 21 day cycle in subjects with refractory or recurrent RMS.
Determine PFS at 4 months.
Determine PFS at 6 months

Secondary objectives of the Expansion Cohort segment of the study are to assess:
Antitumor activity of mocetinostat+vinorelbine in metastatic/refractory RMA as measured by Overall Response Rate (ORR) and Duration of Response (DOR), Disease Control (DC), Duration of Disease Control, as well as
Progression-Free Survival (PFS) According to RECISTv1.1
   ORR includes Complete Response (CR) and Partial Response (PR)
   DOR as defined from the first date a response is identified (either CR or PR) until the date of disease progression
   Disease Control (DC) defined as the proportion of subjects with a confirmed CR, PR or Stable Disease (SD)
   Duration of Disease Controldefined as first date of disease control identified (either CR, PR or SD) until the date of progression
Safety and tolerability of mocetinostat and vinorelbine as characterized by Adverse Event type, severity, timing and relationship to study drug, as well as laboratory abnormalities
Pharmacodynamics of mocetinostat on molecular targets in surrogate tissue
Exploratory biomarker development to enable prediction of drug toxicity, tumor response and the mechanism(s) of acquired study drug resistance.
Obtain RMS tissue biological samples pre-treatment and at progression to assess for differences in gene expression by Next Gen Sequencing and RNA Seq.

Study Design. RMS001 is a single-center, open-label, Phase 1/II study in which the safety and efficacy of mocetinostat in combination with vinorelbine will be evaluated in subjects with locally advanced/unresectable RMS or metastatic or recurrent RMS who have failed front line therapies. Once an appropriate subject has been identified, a 30-day screening period will begin to evaluate eligibility using the defined study inclusion and exclusion criteria.

### Drug Administration

Vinorelbine will be administered at a dose of 25 mg/m2 given intravenously via a central venous catheter on days 1, 8, and 15 in a 21 day cycle. Standard guidelines, including the package insert and institutional protocols, should be followed to calculate the correct dose as well as preparing the dosing solution. Vinorelbine will be used per its FDA approved labeling.
Mocetinostat will be administered orally three times per week beginning on day 3 for a total of 9 doses in a 21 day cycle.
   Mocetinostat should be administered with water.
   Medications that affect gastric pH, including antacids and H2 antagonists, should be avoided 4 hours before and 1 hour after administration of mocetinostat.

Phase 1 Dose Escalation Segment. There will be no dose escalation or de-escalation for vinorelbine. The starting daily dose level for mocetinostat in the dose escalation segment will be 40 mg per dose for cohort 1. Cohort 2 dosing will be mocetinostat 70 mg per dose. Cohort 3 dosing will be mocetinostat 90 mg per dose.

TABLE 1
Phase 1 Dose Escalation Cohort Mocetinostat Dosing

| Cohort | Mocetinostat Dose |
|---|---|
| Cohort 1 | 40 mg/dose |
| Cohort 2 | 70 mg/dose |
| Cohort 3 | 90 mg/dose |

A “3+3” subject enrollment scheme will be followed during the dose escalation. This segment will be performed in sequential cohorts of subjects receiving mocetinostat orally. Cycles will consist of vinorelbine treatment once daily on days 1, 8 and 15 and mocetinostat treatment three times per week beginning on day 3 for a total of 9 doses in a 21 day cycle. If 2 of 3 subjects experience a first-cycle DLT then accrual to the cohort will cease. If a first-cycle DLT is seen in one of the 3 subjects in a cohort, that cohort will enroll an additional 3 subjects. The dose escalation will continue until a first cycle DLT has been observed in 2 of 6 or 2 of 3 subjects. DLT is defined as an adverse event occurring during the first cycle that is at least possibly related to mocetinostat and meets the DLT definition. When 0 of 3, or 1 of 6 subjects in a cohort experience DLT, the dose will be escalated in the subsequent cohort. Dose escalation will begin with dose increase in successive cohorts of 3 subjects until 1 subject experiences a first-cycle DLT (as defined in Table 1); or 2 subjects experience similar AEs that are greater than or equal to grade 2 severity (greater than or equal to grade 3 severity for hematological AEs) which occur during the first cycle.

TABLE 2
Dose Limiting Toxicity

| Category | Criteria |
|---|---|
| Hematology | Grade 4 neutropenia (absolute neutrophil count |
| toxicities | [ANC] <500/mm3) lasting >7 days; |
| | Grade 4 anemia; |
| | Neutropenic Sepsis (ANC <1000/mm3 with |
| | documented serious infection); |
| | Grade ≥3 neutropenic infection (i.e., infection |
| | documented clinically or microbiologically with |
| | grade ≥3 neutropenia); |
| | Grade 4 thrombocytopenia (platelet count <25,000/mm3) |
| | with bleeding; |
| | Grade 3 thrombocytopenia (platelet count <50,000- |
| | 25,000/mm3) lasting >7 days or associated with |
| | clinically significant bleeding. |
| Gastrointestinal | All ≥Grade 4 vomiting or diarrhea; |
| toxicities | Grade 3 nausea or vomiting despite optimal antiemetic |
| | therapy that fails to recover to at least Grade 2 |
| | within 72 hours; |
| | Grade 3 diarrhea despite optimal management of the |
| | event that fails to recover to at least Grade 2 |
| | within 72 hours. |
| CNS toxicities | Grade ≥3 |
| Other | Grade ≥3; |
| nonhemato- | Grade 2 increase in AST/ALT in combination with a |
| logical | grade 2 increase in bilirubin; [symbol] |
| toxicities | For subjects with liver metastases with elevated |
| | liver transaminases at baseline (2.5-5x ULN), |
| | DLT shall be defined as a doubling of the baseline |
| | liver transaminase value(s); |
| Failure to | Failure to recover to Grade ≤2 toxicity or to |
| recover (except | baseline values after delaying the initiation of |
| alopecia) | next cycle by a maximum of 21 days |

The MTD is the dose level at which 0 of 6 or 1 of 6 subjects experience first-cycle DLT, and at least 2 of 3 or 2 of 6 subjects experience first-cycle DLT at the next higher dose level. Effectively, the MTD is the highest dose associated with first-cycle DLT in <33% of subjects.

It is anticipated that 3 cohorts will be completed during the dose escalation segment of this trial (cohort 1:40 mg; cohort 2:70 mg; cohort 3:90 mg). With the concurrence of the investigators and the study sponsor, further testing may be performed in up to 12 additional subjects per dose level to refine the estimation of the MTD and RP2D at intermediate dose levels or to define a higher MTD and RP2D while using Cycle 1 primary supportive care prophylaxis (e.g., with growth factors, antidiarrheals, antiemetics) for bone marrow and/or gastrointestinal toxicities.

After the RP2D has been determined, an expansion cohort will be enrolled.

Expansion Cohort. The expansion cohort segment of this study will consist of 20 additional subjects with refractory alveolar RMS >12 years of age.

All subjects in the expansion cohort will receive the RP2D of Mocetinostat.

Dose reductions. Additional cycles of therapy may be administered provided that the subject meets the following criteria on Day 1 of each cycle:
ANC>1,000/mm3
Hemoglobin >8.0 gm/dL (Blood transfusions are permitted.)
Platelets >50,000/mm3
Non-hematologic toxicity recovered to <Grade 1 (or tolerable Grade 2)

Subjects with toxicities that are manageable with supportive therapy may not require dose reductions (e.g., nausea/vomiting may be treated with antiemetics, anemia may be treated with blood transfusions). Dose de-escalation for subjects may be warranted after Cycle 1 as a consequence of drug-related toxicities. Dose reduction will be documented in the CRF along with reason for reduction.

In the Phase 1 Dose Escalation Cohorts, mocetinostat dose de-escalation of 1-2 levels is allowed but there will be no dose de-escalation below Cohort 1 level dosing of 40 mg. If unacceptable toxicity occurs at the cohort 1 dosing, subjects will be instructed to hold or discontinue treatment.

In the Phase 2 Dose Expansion Cohorts, mocetinostat dose de-escalation of 1-2 levels below the dose chosen for this phase is allowed unless the Cohort 1 (40 mg) dose is chosen. At this point, if a subject experiences unacceptable toxicity, they will be instructed to hold or discontinue treatment.

Doses reduced for drug-related toxicity should not be re-escalated, even if there is minimal or no toxicity with the reduced dose.

For adverse events not specified below, doses may be reduced or held at the discretion of the investigator for the subject's safety. The sponsor should be made aware of such reductions.

TABLE 3
Recommended dose modifications based
on type of AE or laboratory findings:

| AE or lab finding | Dose modification |
|---|---|
| ≥Grade 2 | Subjects experiencing ≥Grade 2 neutropenia |
| neutropenia | may receive G-CSF or other myeloid growth |
| | factors after the first cycle or as defined |
| | in the dose-escalation plan. |
| Grade 3 or 4 | First occurrence- hold mocetinostat and |
| neutropenia | vinorelbine until ANC >1,000/mm3, then |
| | resume mocetinostat and vinorelbine at |
| | same dose. |
| | Second occurrence- hold mocetinostat and |
| | vinorelbine until ANC >1,000/mm3, then |
| | reduce mocetinostat dose to one dose cohort |
| | level lower than the current dose and |
| | resume vinorelbine at same dose |
| | Dose reduction for neutropenia should occur |
| | when the next cycle of study drug is begun. |
| Grade 4 | Any occurrence despite use of G-CSF or |
| neutropenia | other myeloid growth factors - hold |
| lasting longer | mocetinostat and vinorelbine until |
| than 7 days | ANC >1,000/mm3, then reduce mocetinostat |
| | dose to one dose cohort level lower than |
| | the current dose and resume vinorelbine |
| | at the same dose. |
| Grade 3 or 4 | Any occurrence despite use of G-CSF or |
| febrile | other myeloid growth factors - hold |
| neutropenia | mocetinostat and vinorelbine until |
| | ANC >1,000/mm3 and temperature <38 |
| | degrees Celsius, reduce mocetinostat |
| | dose to one dose cohort level lower |
| | than the current dose and resume |
| | vinorelbine at the same dose. |
| Grade 3 or 4 | First occurrence- hold mocetinostat and |
| thrombocytopenia | vinorelbine until platelets ≥50,000/mm3, |
| | then resume mocetinostat and vinorelbine |
| | at same dose. |
| | Second occurrence- hold mocetinostat and |
| | vinorelbine until platelets ≥50,000/mm3, |
| | then reduce mocetinostat dose to one dose |
| | cohort level lower than the current dose |
| | and resume vinorelbine at the same dose. |
| | Use of platelet growth factors or platelet |
| | transfusions is permissible. |
| Hemoglobin <8.0 | Any occurrence- hold mocetinostat and |
| gm/dL | vinorelbine until hemoglobin >8.0 gm/dL, |
| | then resume mocetinostat and vinorelbine |
| | at same dose. |
| | Blood transfusions are permitted. |
| Grade 2 or greater | Any occurrence- hold mocetinostat and |
| non- hematologic | vinorelbine until toxicities have resolved |
| toxicity (unless | or improved to Grade 2 severity levels, |
| clearly and | then resume mocetinostat and vinorelbine |
| incontrovertibly | at same dose if event was a tolerable Grade |
| unrelated to | 2 (at Pi's discretion). Reduce mocetinostat |
| mocetinostat): | dose to one dose cohort level lower than the |
| | current dose if event is Grade 3 or 4 or |
| | intolerable Grade 2 in severity. |
| Management of | In the event of symptoms of cystitis (e.g. |
| Mocetinostat | dysuria, pollakiuria, hematuria, urgency, |
| Associated | or bladder spasm) suspected to be attributable |
| Cystitis | to mocetinostat treatment: |
| | Perform diagnostic evaluation and manage per |
| | institutional standards |
| | If clinically significant symptoms persist |
| | despite a negative diagnostic assessments or |
| | treatment of an associated condition, interrupt |
| | study treatment until resolution of clinically |
| | significant symptoms; and |
| | Resume dosing of mocetinostat when medically |
| | appropriate, |
| Management of | Patients will be assessed for evidence of |
| Mocetinostat | pericardial toxicity during scheduled visits |
| in Event of | according to the Schedule of Assessments |
| Pericardial | The following findings would heighten suspicion |
| Toxicity | of pericardial effusion or pericarditis and |
| | prompt immediate evaluation by ECHO: |
| | Symptoms: shortness of breath, orthopnea, |
| | chest pain, dizziness, rapid pulse |
| | Clinical exam: hypotension, jugular venous |
| | distension, pulsus paradoxus, faint heart |
| | sounds, friction rub, and/or arrhythmia |
| | ECG: sinus tachycardia, atrial fibrillation, |
| | atrial flutter, low voltage with nonspecific |
| | ST-T wave changes and ST elevation or PR |
| | depressions, arrhythmia |
| | ECHOs will be used to assess and categorize |
| | pericardial fluid as minimal (or trivial), |
| | small, moderate or large and will assess for |
| | hemodynamic compromise. |
| | Pericardial effusions will be assessed and |
| | managed as per Table 4 below |

TABLE 4
Pericardial Effusion and Patient Management Guidelines

| Category | Definitions | Patient Management |
|---|---|---|
| Minimal | A small echo-free | De novo (i.e., not |
| (or trivial) | space in the posterior | present at baseline) |
| | atrioventricular groove | pericardial effusion: |
| | that is visible only | Study treatment may |
| | in systole when the | be continued at the |
| | heart has pulled away | discretion of the |
| | from the pericardium. | investigator. |
| | Typically represents | Increased ECHO and |
| | a normal amount of | ECG monitoring weekly |
| | pericardial fluid in | until effusion is no |
| | a disease-free state. | longer present or has |
| | | not progressed over a |
| | | period of 2 weeks. |
| | | Regular assessment |
| | | schedule afterwards. |
| Small | <1 cm of posterior | Study drug will not |
| | echo-free space, with | be discontinued in |
| | or without fluid | these Patients, at the |
| | accumulation elsewhere, | discretion of the |
| | present throughout the | investigator, unless |
| | cardiac cycle, including | the effusion progresses. |
| | diastole (and not only | Increased ECHO and |
| | systole). | ECG monitoring weekly |
| | | for the first month |
| | | after the new effusion |
| | | first noted or until |
| | | the effusion has |
| | | regressed (if sooner). |
| | | Treatment for the |
| | | effusion may be |
| | | administered at the |
| | | discretion of the |
| | | Investigator. |
| Moderate | 1 to 2 cm of echo- | Remove immediately |
| | free space. Moderate | from study treatment. |
| | effusions tend to be | |
| | seen along the length | |
| | of the posterior wall | |
| | but not anteriorly. | |
| Large | >2 cm of maximal | Manage according to |
| | separation. Large | the standard of care |
| | effusions tend to be | at the discretion of |
| | seen circumferentially. | the investigator. |
| | | Refer to cardiologist |
| | | for follow-up as |
| | | clinically indicated, |
| | | until resolution of |
| | | stabilization. |
| Hemodynamic | RV compression, IVC | Remove immediately |
| Compromise | dilation without | from study treatment. |
| | respiratory variation, | Refer to cardiologist |
| | abnormal flow variation | for follow up as |
| | across the AV valves | clinically indicated, |
| | without respiratory | until resolution or |
| | variation, enlarged or | stabilization. |
| | collapsed ventricles. | Collect blood and test |
| | RA diastolic collapse | for anti-nuclear |
| | in isolation is too | antibody (ANA) and |
| | non-specific to signal | anti-histone antibody. |
| | hemodynamic compromise, | |
| | but should be considered | |
| | consistent with this | |
| | diagnosis when accom- | |
| | panied by other findings | |

In exceptional circumstances where ECHO is not considered a technically optimal assessment of pericardial space (e.g., overweight patient), other methods (e.g., MRI) should be used for pericardial assessments. In such cases, the guidelines provided in Table 3 would not apply, and the evaluation should be performed in consultation with the Sponsor. In the event that a pericardial effusion is first identified by a method other than ECHO, efforts should be made to obtain an ECHO for assessment of effusion size.

Additional study assessments. Adverse event (AE) monitoring for subject will begin upon the initiation of mocetinostat and will continue for 28 days after the last administration of mocetinostat. AEs will be graded according to the National Cancer Institute Common Terminology Criteria for Adverse Events Version 5.0 (NCI CTCAE v5.0). For events not reported in the CTCAE, the Investigator will use the grade or adjectives as defined in the Adverse Events section of the study protocol.

Subjects will have tumor assessment performed approximately every 6 weeks (+/−1 week), beginning from the initiation of mocetinostat. Tumor assessments will cease if the subject is determined to meet the criteria for progressive disease or begins a different cancer therapy or withdraws consent. An End of Treatment Visit will be conducted either at the Day 22 visit or within 7 days after the end of last cycle. If the subject discontinues prior to completing mocetinostat within a cycle, the Early Termination visit should be done within 2 days of discontinuation. A Safety Follow-Up telephone call will be conducted approximately 28 days following the last day of mocetinostat. Additionally, each subject will be contacted by telephone or email approximately every 3 months following study discontinuation until death, loss to follow-up, or withdrawal of consent in order to assess disease progression status. Safety will be monitored via laboratory assessments, physical examinations, electrocardiograms (ECG), vital signs, and AEs. Study assessments will be performed as per Schedule of Events (see Tables at end of the synopsis).

Subject Eligibility

Inclusion criteria Subjects who meet the following inclusion criteria will be eligible to participate in this study:
1. Ages 13-35 years old
2. Willing and able to provide written IRB/IEC-approved Informed Consent. For subjects <18 years of age, their parents or legal guardians must sign a written informed consent. Assent, when appropriate, will be obtained according to institutional guidelines.
3. Have histologically or cytological confirmed diagnosis of Rhabdomyosarcoma with locally advanced/unresectable, metastatic, refractory or relapsed disease who have failed standard therapy and for whom no known curative therapy exists.
4. Measurable disease according to RECIST version 1.1.
5. Prior cancer therapy: Subjects may have received any number of prior therapy regimens. In the Investigator's opinion, subjects must have tolerated prior cytotoxic therapies well and have adequate bone marrow reserve. At the time of treatment initiation, at least 3 weeks must have elapsed after prior cytotoxic chemotherapy. At least 7 days must have elapsed since completion of any prior non-cytotoxic cancer therapy and any associated AEs must have resolved.
6. Prior radiotherapy is allowed if ≥2 weeks have elapsed for local palliative XRT (small port); ≥6 months must have elapsed if prior total body irradiation, craniospinal XRT or if >50% radiation of the pelvis; >6 weeks must have elapsed if other substantial bone marrow radiation (defined per PI's discretion). Subjects who have received brain irradiation must have completed whole brain radiotherapy and/or gamma knife at least 4 weeks prior to enrollment.
7. Subjects with controlled asymptomatic CNS involvement are allowed in absence of therapy with anticonvulsants. Subjects not requiring steroids or requiring steroids at stable dose (≤4 mg/day dexamethasone or equivalent) for at least 2 weeks are eligible.
8. Resolution of all acute toxic effects (excluding alopecia) of any prior anti-cancer therapy to NCI CTCAE (Version 4.03) Grade <1 or to the baseline laboratory values as defined in the table below.
9. Eastern Cooperative Oncology Group (ECOG) performance status (PS)≤2 in subjects ≥17 years old; or Karnofsky/Lansky >50 in subjects <16 years old.
10. Subjects age >18 years for first cohort. Subjects must be >12 years old for the second and subsequent cohorts
11. Life expectancy of at least 3 months.
12. Baseline laboratory values fulfilling the following requirements:

| | |
|---|---|
| Absolute Neutrophil Count | ≥1000/mm3 (≥1.0 × 109/L) |
| (ANC) | |
| Platelets (PLT) | ≥100,000/mm3 (≥100 × 109/L) |
| | (transfusion independent, defined |
| | as not receiving platelet transfusions |
| | within a 7 day period prior to screening) |
| Hemoglobin >9.0 g/dL | >9.0 g/dL (transfusions are allowed) |
| (transfusions are allowed) | |
| Serum Creatinine | ≤1.5 × ULN |
| Or | |
| Creatinine Clearance | >60 mL/min |
| Total Serum Bilirubin | ≤1.5 × ULN; ≤5 × ULN if Gilbert's |
| | Syndrome |
| Liver Transaminases | ≤2.5 × ULN; ≤5 × ULN if liver |
| (AST/ALT) | metastases are present. |
| Pregnancy test if female | Negative within 7 days of starting treatment |
| of child-bearing potential | |

AST/ALT = aspartate aminotransferase/alanine aminotransferase, ULN = upper limit of normal
Growth factor(s): Growth factors that support platelet or white cell number or function must not have been administered within the 7 days prior to screening.

13. Cardiac ejection fraction >50% or shortening fraction >28% by ECHO or MUGA.
14. Females of child-bearing potential must have a negative pregnancy test during screening and be neither breastfeeding nor intending to become pregnant during study participation. Females of childbearing potential must agree to avoid pregnancy during the study and commit to abstinence from heterosexual intercourse or agree to use two methods of birth control (one highly effective method and one additional effective method) at least 4 weeks before the start of protocol therapy, for the duration of study participation, and for 6 months after the last dose of mocetinostat.
15. Males with partner(s) of childbearing potential must take appropriate precautions to avoid fathering a child from the screening period until 90 days after receiving the last dose of mocetinostat. They must commit to abstinence from heterosexual intercourse or agree to use appropriate barrier contraception.
16. Prior to enrollment of females or males of reproductive potential, the investigator must document confirmation of the subject's understanding of the possible teratogenic effects of mocetinostat.
17. Willingness and ability to comply with scheduled visits, treatment plan, laboratory tests and other study procedures.

Exclusion criteria. Subjects will not be enrolled if they meet any of the following exclusion criteria:
1. Current participation in another therapeutic clinical trial.
2. Symptomatic brain metastases.
3. History of previous cancer (non RMS), except squamous cell or basal-cell carcinoma of the skin or any in situ carcinoma that has been completely resected, which required therapy within the previous 3 years. Other low grade cancers can be reviewed and allowed at the discretion of the PI.
4. Incomplete recovery from any surgery (other than central venous catheter or port placement) prior to treatment.
5. Any of the following in the past 6 months: pericarditis, pericardial effusion, symptomatic congestive heart failure, cerebrovascular accident or transient ischemic attack, pulmonary embolism, deep vein thrombosis, symptomatic bradycardia, requirement for anti-arrhythmic medication.
6. History of prolonged QTc interval (e.g., repeated demonstration of a QTc interval >450 milliseconds, unless associated with the use of medications known to prolong the QTc interval). QTc will be calculated using the Bazett formula (RR interval=60/heart rate; QTI Corrected=QT interval/sqr (RRinterval)).
7. History of additional risk factors for torsade de pointes (e.g., heart failure, family history of long QT syndrome).
8. Use of concomitant medications that increase or possibly increase the risk to prolong the QTc interval and/or induce torsades de pointes ventricular arrhythmia.
9. Females who are breastfeeding/lactating.
10. Known active infections (e.g., bacterial, fungal, viral including hepatitis and HIV positivity).
11. Other severe acute or chronic medical or psychiatric condition or laboratory abnormality that may increase the risk associated with study participation or study drug administration or may interfere with the interpretation of study results and, in the judgment of the Investigator, would make the Subject inappropriate for entry into this study or compromise protocol objectives in the opinion of the Investigator and/or the Sponsor.

### Study Plan

Enrollment, treatment assignment and dose escalation. Subjects will be screened within 30 days prior to enrollment to confirm that they meet the eligibility criteria specified in section 5 of this protocol and as determined by the local PI. Subjects are considered enrolled once they sign the informed consent form (ICF). However, an enrollment number will not be assigned until the subject is determined to be eligible. Each subject at each study center who meet the inclusion/exclusion criteria will be assigned an enrollment number. Subjects will be assigned to dose cohorts of 3 subjects per dose cohort. In the phase 1 portion of the study, the dose of the initial dose cohort will be 40 mg mocetinostat. Dose escalation will continue until 1 subject experiences a first-cycle DLT (as defined in Section 4, Study Design); or 2 subjects experience AEs that are greater than or equal to grade 2 severity which occur during the first cycle. If the criteria for stopping dose escalation are not met, doses will then be escalated in subsequent cohorts. Each dose cohort will include a minimum of 3 evaluable subjects for assessment of toxicity in their first cycle. Subsequent cohorts will not be enrolled until 3 subjects in the previous cohort have completed their first cycle. In any cohort, if 1 subject experiences a first-cycle DLT, 3 additional subjects will be enrolled at that dose level. If 2 of 3 or 2 of 6 subjects experience a first-cycle DLT, the maximum administered dose will have been defined and any further dose-finding will be performed as defined in Section 4.2.

The decision to dose escalate will be made by agreement between the PIs and representatives of the Sponsor and will be based on toxicities. The safety of each cohort will be reviewed prior to the start of the next cohort. The sponsor and PIs will review toxicities on a routine basis via a teleconference call and discuss dose escalations. The frequency of discussions will depend on the enrollment of each of the cohorts.

Study assessments. This section describes study assessment procedures. Please refer to the Synopsis and Section 6.3 for the schedules of assessments.

### Pharmacokinetic Assessments

Blood sample collections for plasma pharmacokinetic assays of mocetinostat. PK samples will be obtained to determine the systemic exposure to mocetinostat. Drug concentration in plasma will be measured using a validated LC-MS/MS method. PK parameters such as Css, CL, Vd, and t1/2 will be calculated for each subject using non-compartmental methods.

For the dose escalation segment of the study, blood samples will be collected at the following times for all subjects in each cohort: For the first treatment cycle, blood samples for PK analysis will be drawn pre-dose, and at 1, 3, and 7 hours after the cycle 1, day 14 dose of mocetinostat.

Instructions for collecting, processing, storing and shipping the pharmacokinetic samples will be provided in the laboratory manual.

Pharmacodynamic assessments. Pharmacodynamic studies will be conducted on blood samples obtained from all subject to determine potential biomarkers. Archived tumor samples may be requested at a future date for exploratory biomarkers and/or other analyses.

Pharmacodynamic blood samples will be collected at the following times:
on cycle 1 day 1 prior to vinorelbine dosing;
on cycle 1, day 14 prior to mocetinostat dosing and 3 hours after mocetinostat dosing;
at end of the study.

### Safety Assessments

Physical examination. A complete physical examination will include the following: HEENT (head, ears, eyes, nose and throat), chest, lungs, heart, lymph nodes, abdomen, skin, musculoskeletal and neurological systems. Symptom-driven physical exams will be done as needed, based on observed adverse events.

Height and weight. Height and weight will be measured during screening. Weight only will be measured at subsequent visits.

Electrocardiogram. An electrocardiogram (ECG) will be obtained using a 12-lead electrocardiogramach ECG is to be evaluated by the study investigator for the presence of abnormalities at the time the ECG is recorded. The evaluating physician is to write his/her diagnosis on the ECG recording and sign and date. In some cases, it may be useful to repeat abnormal ECG's to rule out improper lead placement as contributing to the ECG abnormality.

Echocardiogram. An echocardiogram (or MUGA) will be done during the screening period, within 1 week prior to starting cycles 2, 3 and 4, and at the end of study in order to determine cardiac function and to evaluate for pericardial effusion. If the screening ECHO (or MUGA) is done within 1 week of starting cycle 1, day 1, then it does not need to be repeated in order to start cycle 1, day 1.

Vital signs. Vital signs will include:
blood pressure, respiratory rate, heart rate (with the subject in the sitting position following an approximate 5-minute rest)
temperature (° C.)

Clinical laboratory tests. All screening clinical laboratory test results are to be reviewed and assessed by the investigator, or designee, prior to study enrollment. Any screening laboratory result is required for eligibility that is outside the reference range as allowed by entry criteria may be repeated, as deemed necessary by the Principal Investigator. If any repeat screening values continue to be outside the reference range, the subject will be excluded from the study, unless there is agreement between the sponsor and investigator that the laboratory deviation is not clinically significant. After enrollment into the study, the CBC, serum electrolytes, BUN, creatinine, liver function tests and urinalysis are to be reviewed and assessed for safety prior to drug administration on Day 1 of each treatment cycle.

Blood samples for the following tests will be collected:
Hematology: complete blood count (CBC) including WBC with differential (neutrophils, lymphocytes, monocytes, eosinophils, basophils) RBC, hemoglobin, hematocrit, and platelet count.
Clinical chemistry: serum sodium, potassium, chloride, bicarbonate, BUN, creatinine, glucose, calcium, total bilirubin, direct (conjugated) bilirubin, indirect (unconjugated) bilirubin (calculated), alanine transaminase (ALT), aspartate transaminase (AST), alkaline phosphatase, protein, albumin.

Urine will be collected for the following tests:
Urinalysis: color, protein, glucose, bilirubin, ketones, blood, pH, specific gravity, leukocytes and leukocyte esterase.

### Other Assessments

Urine pregnancy test. Urine pregnancy tests will be conducted for all females of child bearing potential.

Demographics. Information regarding the subject's gender, age, and racial or ethnic origin will be collected.

Concomitant medications. At each clinical assessment, subjects will be monitored for the use of concomitant medications during the study. Subjects will be asked if they used any medications (prescription or over the counter), and herbal or dietary supplements since their previous assessment.

Therapies considered necessary for the subject's well-being (e.g., to manage chronic pathologies or therapies required for life-threatening medical conditions) may be administered at the discretion of the Investigator.

Mocetinostat is metabolized by CYP2E1 and CYP3A and possibly by CYP2C8 and CYP2C19. Mocetinostat is considered to be a strong CYP2C9 inhibitor. To minimize the influence of potential drug-drug interactions on the identification of the maximum tolerated dose of mocetinostat, the concomitant use of medications that are strong inhibitors or inducers of CYP2E1, CYP3A, CYP2C8 or CYP2C19 should be avoided if possible. Additionally, medications that are significantly metabolized by CYP2C9, should be used with caution or replaced with other agents not significantly metabolized by CYP2C9.

Particular attention should be paid to subjects receiving warfarin.
Strong CYP3A inhibitors: boceprevir, cobicistat, conivaptan, danoprevir and ritonavir, elvitegravir and ritonavir, grapefruit juice, indinavir and ritonavir, itraconazole, ketoconazole, lopinavir and ritonavir, paritaprevir and ritonavir and (ombitasvir and/or dasabuvir), posaconazole, ritonavir, saquinavir and ritonavir), telaprevir tipranavir and ritonavir, troleandomycin, voriconazole
Strong CYP3A inducers: cabamazepime, enzalutamide, mitotane, phenytoin, rifampin, St. John's wort
Strong CYP2C8 inhibitors: clopidogrel, gemfibrozil
Strong CYPC19 inhibitors: fluconazole, fluoxetine, fluvoxamine, ticlopidine
Strong CYPC19 inducers: rifampin, ritonavir
CYP2C9 sensitive index substrates: tolbutamide, S-warfarin

Mocetinostat is a substrate and an inhibitor of P-gp. Thus, P-gp sensitive substrates and strong inhibitors of P-gp should be used with caution or replaced with other agents.
P-gp substrates: dabigatran, digoxin, fexofenadine
P-gp inhibitors: amiodarone, carvedilol, clarithromycin, dronedarone, itraconazole, lapatinib, lopinavir and ritonavir, propafenone, quinidine, ranolazine, ritonavir, saquinavir and ritonavir, telaprevir, tipranavir and ritonavir, verapamil

Clinically significant drug interactions have been reported when using vinorelbine with strong CYP450 3A4 inhibitors and inducers. Thus, the concomitant use of vinorelbine with these agents should be avoided if possible.
Strong CYP450 34A inducers: Apalutamide, Carbamazepine, Enzalutamide, Fosphenytoin, Lumacaftor, Mitotane, Phenobarbital, Phenytoin, Primidone, Rifampin.
Strong CYP450 34A inhibitors: Atazanavir, Boceprevir, Clarithromycin, Cobicistat and cobicistat containing coformulations, Darunavir, Idelalisib, Indinavir, Itraconazole, Ketoconazole, Lopinavir, Mifepristone, Nefazodone, Nelfinavir, Ombitasvir-paritaprevir-ritonavir, Ombitasvir-paritaprevir-ritonavir plus dasabuvir, Posaconazole, Ritonavir and ritonavir containing coformulations, Saquinavir, Telaprevir, Telithromycin, Voriconazole

Medical History. The subject's medical history will be taken with particular attention to questions related to 1) a thorough review of body systems including any past or current conditions; 2) previous and current pharmacotherapy or chronic use of any medication within 14 days prior to screening; and 3) history of allergies or hypersensitivity to drugs.

Performance status. The ECOG performance status (in subjects ≥17 years old) or the Karnofsky/Lansky status (in subjects ≤16 years old) will be assessed at Screening and at the beginning of each cycle.

Tumor assessments. Tumor assessments will be performed from evaluation of a CT or an MRI scan at screening and end of cycles 2, 4, 6, etc. (i.e., every 6 weeks)+/−1 week. The same methodology should be used for all assessments throughout the study. Assessments will be done using RECIST, version 1.1. Tumor assessments will continue to be done after the subject discontinues from the study, until the criteria for progressive disease have been met, or the subject begins another cancer therapy, or withdraws consent.

Adverse events. An adverse event (AE) is any untoward medical occurrence in a subject administered a study drug, and that does not necessarily have a causal relationship with the study drug. (An AE can be any unfavorable and unintended sign or symptom, or disease temporally associated with the use of a study drug, whether or not related to the study drug.) Adverse events include any symptom, physical sign, syndrome or disease which either occurs during the study, having been absent at baseline, or, if present at baseline, appears to worsen during the course of a clinical trial, after starting treatment, whether considered treatment related or not.

Events involving adverse drug reactions, illnesses with onset during the study, or exacerbations of pre-existing illnesses should be recorded. Exacerbation of a preexisting illness is defined as a manifestation (sign or symptom) of the illness that indicates a significant increase in the severity of the illness as compared to the severity noted at the start of the study. It may include a worsening or increase in severity of signs or symptoms of the illness, increase in the frequency of signs and symptoms of an intermittent illness, or the appearance of a new manifestation/complication. Exacerbation of a pre-existing illness should be considered when a subject requires new or additional concomitant therapy for the treatment of that illness during the study. Lack of, or insufficient clinical response, benefit, efficacy, therapeutic effect, or pharmacologic action, should not be recorded as an adverse event. The investigator must make the distinction between exacerbation of a pre-existing illness and lack of therapeutic effect.

For all adverse events, the investigator must pursue and obtain information adequate to determine both the outcome of the adverse event and to assess whether it meets the criteria for classification as a serious adverse event requiring immediate notification to the sponsor or its designated representative. For all adverse events, the investigator is required to obtain sufficient information to assess the causality of the adverse event (i.e., study drug or other illness). Follow-up of the adverse event, after study drug has been discontinued, is required if the adverse event or its sequelae persist. Follow-up of all adverse events is required until the event or its sequelae resolve or stabilize at a level acceptable to the investigator and the sponsor or its designated representative.

Adverse events may be volunteered spontaneously by the subject or be discovered as a result of general questioning by the investigator or by physical examination or laboratory tests. Subject s will be continuously monitored for adverse events (AEs) during the study. At each visit during the study or at each telephone contact after the first dose, subject s will be asked to specifically describe any signs, symptoms, or AEs occurring since the previous visit. Questions will be phrased so that they do not “lead” the subject into giving information that is not valid. All adverse events regardless of treatment group or suspected causal relationship to study drug will be recorded in source documentation and on the adverse event page(s) of the case report form (CRF).

Conditions that the subject experienced before treatment with study drug (Pre-dose symptoms) and any new signs, symptoms, or AEs that occur since starting treatment with study drug (regardless of causality) are to be assessed and recorded. Pre-dose baseline assessments must be performed prior to treatment with mocetinostat. Any baseline symptoms or adverse events noted to treatment with mocetinostat should be recorded as part of the Medical History. The assessment and recording of each symptom or AE must also be described by its duration (start date, time and duration), its severity (mild, moderate, severe, very severe), its relationship to the study medication (unrelated, unlikely or possibly related), whether it influenced the course of the study medication, and whether it required specific therapy.

The severity of signs, symptoms, or AEs is to be determined by using the NCI Common Terminology Criteria for Adverse Events (CTCAE) v 5.0. If a sign, symptom or AE is not included in the toxicity severity grading scale, the intensity of the event will be graded as shown below.
mild (grade 1): Symptoms causing no or minimal interference with usual social and functional activities. Symptoms are usually transient and require no special treatment.
moderate (grade 2): Symptoms causing greater than minimal interference with usual social and functional activities. Symptoms are usually ameliorated by simple therapeutic measures.
severe (grade 3): Symptoms causing inability to perform usual social and functional activities. Symptoms traditionally require systemic drug therapy or other treatment
very severe (grade 4, life-threatening): Symptoms causing inability to perform basic self-care functions or require medical or operative intervention to prevent permanent impairment, persistent disability, or death.
death related to AE (grade 5)

Assessment of causal relationship. A medically-qualified investigator must assess the relationship of any AE to the use of study drug, based on available information, using the following guidelines:
Not related: There is not a reasonable causal relationship to the investigational product and the adverse event.
Unlikely related: No temporal association or the cause of the event has been identified, or the drug or biologic cannot be implicated.
Possibly related: There is reasonable evidence to suggest a causal relationship between the drug and adverse event.
Related: There is evidence to suggest a causal relationship, and the influence of other factors is unlikely.

Expectedness of an adverse event. The expectedness of an adverse event or suspected adverse reaction shall be determined according to the specified reference document containing safety information (e.g., most current investigator's brochure or product label). Any AE that is not identified in nature, severity, or specificity in the current study drug reference document(s) (e.g., investigator's brochure or FDA package insert) is considered unexpected.

Events that are mentioned in the investigator's brochure as occurring with a class of drugs or as anticipated from the pharmacological properties of the drug, but not specifically mentioned as occurring with the particular drug under investigation are considered unexpected.

Abnormal clinical test findings. Any clinically significant changes in physical examination findings and abnormal objective test findings (e.g., laboratory, x-ray, ECG) should also be recorded as adverse events. The criteria for determining whether an abnormal objective test finding should be reported as an adverse event are as follows:
1. test result is associated with accompanying symptoms, and/or
2. test result requires additional diagnostic testing or medical/surgical intervention, and/or
3. test result leads to a change in study dosing or discontinuation from the study significant additional concomitant drug treatment or other therapy, and/or
4. test result leads to any of the outcomes included in the definition of a serious adverse event, and/or
5. test result is considered to be an adverse event by the investigator or sponsor

Merely repeating an abnormal test, in the absence of any of the above conditions, does not meet condition #2 above for reporting as an adverse event. If additional diagnostic testing (condition #2) is performed to rule out a potential problem/abnormality, and if the test does not confirm the problem/abnormality, the abnormal laboratory result for which diagnostic testing was performed would not be considered an adverse event.

All clinically important abnormal test results occurring during the study will be repeated at appropriate intervals until the abnormal result returns either to baseline or to a level deemed acceptable by the investigator and the sponsor (or its designated representative), or until a diagnosis that explains the abnormal result is made.

Any abnormal test result that is determined to be an error does not require reporting as an adverse event, even if it did meet 1 of the above conditions except for condition #4.

Serious adverse events. All serious adverse events (SAEs) (defined below) regardless of treatment group or suspected relationship to study drug must be reported immediately (within 1 working day) by telephone to the sponsor or its designated representative (see Appendix 1).

An SAE is defined as an adverse event that:
A life-threatening event is any AE that places the subject at immediate risk of death from the reaction/event as it occurred (i.e., it does not refer to an AE that, had it occurred in a more severe form, might have caused death).
Disability is defined as a substantial disruption of a person's ability to conduct normal life functions.
Inpatient hospitalization is defined as any inpatient admission, regardless of duration. For chronic or long-term inpatients, inpatient admission also includes transfer within the hospital to an acute/intensive care inpatient unit. Inpatient admission in the absence of a precipitating, treatment-emergent, adverse event may meet criteria for “seriousness”, but should not be considered or reported as a serious adverse event (e.g., admission for treatment of a pre-existing condition not associated with the development of a new adverse event or with a worsening of a pre-existing condition; social admission (e.g., subject has no place to sleep); administrative admission (e.g., for an annual physical exam); protocol-specified admission during a clinical study (e.g., for a procedure required by protocol); optional admission not associated with a precipitating clinical adverse event (e.g., for elective cosmetic surgery). In addition, inpatient admission does not include any of the following: A visit to the emergency room or hospital clinic; outpatient, same day, ambulatory procedures; observation or short-stay units; rehabilitation facilities; hospice facilities, respite care (e.g., care-giver relief); skilled nursing facilities; nursing homes; custodial care facilities; or clinical research, Phase 1 units.
Prolongation of hospitalization is defined as any extension of an inpatient hospitalization beyond the stay anticipated/required in relation to the original reason for the initial admission. For protocol-specified hospitalizations in clinical trials, prolongation is defined as any extension beyond the length of stay required by protocol. Prolongation in absence of a precipitating, treatment-emergent, adverse event may meet criteria for “seriousness”, but should not be considered or reported as a serious adverse event.
An important medical event is defined as any adverse event that may not be immediately life threatening or result in death or hospitalization, but may jeopardize the subject and may require medical or surgical intervention in order to prevent the event from becoming a serious adverse event, as determined by appropriate medical judgment. Examples of such medical events include; allergic bronchospasm requiring intensive treatment in an emergency room or other setting, blood dyscrasias or convulsions that do not result in inpatient hospitalization, or development of a drug dependency.
Is a congenital anomaly/birth defect.
Results in death.

Study sites will document all SAEs that occur (whether or not related to study drug) per UCLA OHRPP Guidelines. The collection period for all SAEs will begin after informed consent is obtained and end after procedures for the final study visit have been completed. In accordance with the standard operating procedures and policies of the local Institutional Review Board (IRB)/Independent Ethics Committee (IEC), the site investigator will report SAEs to the IRB/IEC.

Non-serious adverse events. For this study, all non-serious adverse events occurring from the start of Cycle 1 Day 1 through the last follow-up visit required by protocol, or 28 days after the last administration of study drug, whichever comes later, will be collected regardless of treatment group or suspected relationship to study drug.

Reporting a pregnancy. Pregnancy occurring during a clinical investigation, although not considered an SAE, must be reported within the same timelines as an SAE. The positive pregnancy test will be recorded in the source and applicable case report form(s). The pregnancy will be followed until final outcome. Any associated AEs or SAEs that occur to the mother or fetus will be recorded as AE or SAE as applicable. If a pregnancy occurs in the partner of a subject participating in the trial, the same guidelines apply.

Schedule of Study Assessments. A flow chart of study assessments is provided in the study synopsis and the descriptions for study assessments are in this section.

Screening Assessments All Cohorts. The screening assessments for all Cohorts in the study will be completed and the results evaluated by the study investigator within 30 days prior to the start of dosing.

The procedures listed below will be performed:
informed consent: must be obtained prior to performing any study related procedure and may be obtained within 30 days prior to starting treatment; for subjects <18 years of age, their parent or legal guardian must sign a written informed consent. Assent, when appropriate, will be obtained according to institutional guidelines;
demographics;
medical history (any events up to the start of medications);
A previously documented CT scan or MRI may be used if obtained within the 30 days prior to treatment;
ECOG or Karnofsky/Lansky performance status;
vital signs;
height and weight;
complete physical examination;
12-lead ECG;
Echocardiogram (or MUGA);
serum or urine pregnancy test (female subjects of child bearing potential only);
clinical laboratory tests (hematology, clinical chemistries, urinalysis);
pharmacodynamic samples;
concomitant medication assessment

Phase 1 Dose Escalation cohorts; Day 1 though End of Cycle 1 and subsequent cycles

### Day 1, Cycle 1 and Subsequent Cycles Assessments, Outpatient Visit

On Day 1 of dose escalation Cohorts 1, 2 and 3, subjects will be seen in the outpatient setting to prepare for the initiation of dosing. The following procedures will be performed:
ECOG or Karnofsky/Lansky performance status**;
vital signs;
height and weight**;
complete physical examination**;
12-lead ECG;
ECHO (or MUGA) within 1 week prior to day 1 of cycles 2, 3, and 4;
Clinical laboratory tests (hematology, clinical chemistries, urinalysis)**;
urine pregnancy test (women of childbearing potential only)**;
concomitant medication assessment;
adverse event assessment;
administration of vinorelbine
a CT scan or MRI will be done every 6 weeks during the study (end of Cycles 2, 4, 6, etc.)+/−1 week. The same method of assessing disease must be used throughout the study.
** If the screening assessments for ECOG or Karnofsky/Lansky performance status, height, weight, physical examination, clinical laboratory tests and urine pregnancy test were performed within approximately 72 hours prior to dosing on Day 1 of Cycle 1, these assessments do not need to be repeated except as required by institutional standards.

Day 8, Cycle 1 and subsequent cycle assessments, Outpatient Visit. On Day 8 (+/−3 days) of Cycle 1 of dose escalation Cohorts 1, 2 and 3, the procedures listed below will be performed during an outpatient visit:
vital signs;
weight;
symptom-driven physical examination;
clinical laboratory tests (hematology, clinical chemistry);
concomitant medication check;
adverse event assessment.
administration of vinorelbine

Day 14, Cycle 1 only, Outpatient Visit. On Day 14 of Cycle 1 of dose escalation Cohorts 1, 2 and 3, the procedures listed below will be performed during an outpatient visit:
the administration of mocetinostat should be initiated early in the day on Day 14, in order to facilitate collection of pharmacokinetic samples (see below);
plasma PK samples on day 14 of cycle 1 only: pre-dose of mocetinostat and 1 hour, 3 hours and 7 hours post-dose
plasma PD samples on day 14 of cycle 1 only: pre-dose of mocetinostat and 3 hours post-dose

Day 15, Cycle 1 and subsequent cycle assessments, Outpatient Visit. On Day 15 (+/−3 days) of Cycle 1 of dose escalation Cohorts 1, 2 and 3, the procedures listed below will be performed during an outpatient visit:
vital signs;
weight;
symptom-driven physical examination;
clinical laboratory tests (hematology, clinical chemistry);
concomitant medication check;
adverse event assessment.
administration of vinorelbine

Phase 2 Expansion Cohort 4; Cycles 1 and subsequent cycles

Day 1 of Cycle 1 and subsequent cycle assessments, Outpatient Visit. The procedures listed below will be performed during an outpatient visit:
ECOG or Karnofsky/Lansky performance status**;
vital signs;
weight**
complete physical examination**;
12-lead ECG;
ECHO (or MUGA) at screening and within 1 week prior to day 1 of cycles 2, 3, and 4;
Clinical laboratory tests (hematology, clinical chemistries, urinalysis)**;
urine pregnancy test (women of childbearing potential only)**; concomitant medication assessment;
adverse event assessment;
administration of vinorelbine
a CT scan or MRI will be done every 6 weeks during the study (end of Cycles 2, 4, 6, etc.) +/−1 week. The same method of assessing disease must be used throughout the study;
** If the screening assessments for ECOG or Karnofsky/Lansky performance status, height, weight, physical examination, clinical laboratory tests and urine pregnancy test were performed within approximately 72 hours prior to dosing on Day 1 of Cycle 1, these assessments do not need to be repeated, except as required by institutional standards.

Day 8, Cycle 1 and subsequent cycle assessments, Outpatient Visit. On Day 8 (+/−3 days) of Cycle 1 of dose escalation Cohorts 1, 2 and 3, the procedures listed below will be performed during an outpatient visit:
vital signs;
weight;
symptom-driven physical examination;
clinical laboratory tests (hematology, clinical chemistry);
concomitant medication check;
adverse event assessment
administration of vinorelbine

Day 14, Cycle 1 only, Outpatient Visit. On Day 14 of Cycle 1 of dose escalation Cohorts 1, 2 and 3, the procedures listed below will be performed during an outpatient visit:
the administration of mocetinostat should be initiated early in the day on Day 14, in order to facilitate collection of pharmacokinetic samples (see below);
plasma PK samples on day 14 of cycle 1 only: pre-dose of mocetinostat and 1 hour, 3 hours and 7 hours post-dose
plasma PD samples on day 14 of cycle 1 only: pre-dose of mocetinostat and 3 hours post-dose

Day 15, Cycle 1 and subsequent cycle assessments, Outpatient Visit. On Day 15 (+/−3 days) of Cycle 1 of dose escalation Cohorts 1, 2 and 3, the procedures listed below will be performed during an outpatient visit:
vital signs;
weight;
symptom-driven physical examination;
clinical laboratory tests (hematology, clinical chemistry);
concomitant medication check;
adverse event assessment
administration of vinorelbine

6.3.4. Early Termination/End of Treatment Visit, All Cohorts, All Cycles. The following procedures will be performed at the time a subject is discontinued from the study, if prior to completing a cycle. This visit should be performed within 3 days of discontinuation. The End of Treatment Visit will be done if a subject completes a cycle but does not continue to the next cycle. This visit should be performed either at the last visit in the cycle (Day 21) or within 7 days after the completion of the cycle. The procedures listed below will be done:
ECOG or Karnofsky/Lansky performance status
vital signs;
weight;
complete physical examination;
12-lead ECG;
ECHO (or MUGA);
clinical laboratory tests (hematology, clinical chemistries, urinalysis);
urine pregnancy test: women of child-bearing potential only;
if a CT scan or MRI is due, it should be done at this visit. The same method of assessing disease must be used throughout the study;
concomitant medication check;
adverse event assessment.

6.3.11 Post-treatment follow-up. A Safety Follow-Up telephone call will be conducted approximately 28 days following the last day of mocetinostat administration; or approximately 28 days after the completion of the last cycle which the subject completes. Additionally, each subject will be contacted by telephone or email approximately every 3 months following study discontinuation until death, loss to follow-up, or withdrawal of consent in order to assess disease progression and survival status.

6.4 Subject withdrawal criteria and procedures. A subject will be permanently discontinued from study treatment if the subject develops a toxicity or concurrent illness that, in the investigator's judgment, precludes further treatment with the study drug.

A subject may be permanently discontinued from the study for any of the following reasons:
Intolerable adverse event that does not improve with dose adjustments
significant abnormal clinical laboratory values that are possibly, probably or certainly attributed to study drug. This includes Grade 3 or greater laboratory abnormalities that do not improve to ≤ Grade 1 or baseline severity within 4 weeks of the last dose received.
pregnancy
non-compliance (not completing all required assessments at required times, missing scheduled visits, high frequency of missed doses, refusing to complete duration of dosing) or protocol violation
subject withdraws consent and refuses to participate in the study
investigator's or sponsor's decision that withdrawal is in the subject's best interest termination of the study by the sponsor (see Section 10.7)
RECIST, version 1.1-defined disease progression is observed If for any reason, a subject is discontinued from study before the subject completes 1 cycle of treatment; all assessments outlined in the Flow Chart of Study Assessments in the Study Synopsis and listed in Section 6.3.3 are to be completed. The reason(s) for a subject's discontinuation of treatment or withdrawal from the study will be clearly documented in the source documents and on the CRF. Any subject who completes Cycle 1 will be considered to have completed the study.

### Study Drugs

### Pharmaceutical Information

### Mocetinostat

Description Mocetinostat is a small molecule HDAC inhibitor that targets human HDAC isoforms.

Dosage form, strength and route of administration. Mocetinostat is formulated as 20 mg and 50 mg hard gelatin capsules.

The starting daily dose level for mocetinostat in the dose escalation segment will be 40 mg per dose for cohort 1. Cohort 2 dosing will be mocetinostat 70 mg per dose. Cohort 3 dosing will be mocetinostat 90 mg per dose. The MTD is the dose level at which 0 of 6 or 1 of 6 subjects experience first-cycle DLT, and at least 2 of 3 or 2 of 6 subjects experience first-cycle DLT at the next higher dose level. Effectively, the MTD is the highest dose associated with first-cycle DLT in <33% of subjects.

### Phase 1 Dose Escalation Cohort Mocetinostat Dosing:

| Cohort | Mocetinostat Dose |
|---|---|
| Cohort 1 | 40 mg/dose |
| Cohort 2 | 70 mg/dose |
| Cohort 3 | 90 mg/dose |

### Phase 2 Dose Expansion Dosing:

The RP2D may be determined by the MTD or optimal target inhibition with an acceptable safety profile.

Supply and stability information. The composition of the drug product consists of a blend of mocetinostat free base drug substance, microcrystalline cellulose (Avicel® PH112), sodium starch glycolate, colloidal silicon dioxide and magnesium stearate (non-bovine).

The composition of each dosage form is shown in Table 2.

TABLE 2
Dosage Forms and Composition of Mocetinostat Formulations

| | Formulatio | | | |
|---|---|---|---|---|
| | Mocetinostat | | | |
| | Dosage Forms | | | |
| | 10 mg | 25 mg capsule | 20 mg capsule | 50 mg capsule |
| Appearance: | White to off- | White to off- | White to off- | White to off- |
| | white powder | white powder | white powder | white powder |
| | in a White | in Swedish | in a White | in Swedish |
| | Opaque hard | Orange hard | Opaque hard | Orange hard |
| | gelatin | gelatin | gelatin | gelatin |
| | capsules 1 | capsules 1 | capsules 1 | capsules2 |
| Composition: | API: Mocetinostat free base | | | |
| Package Size: | 48 capsules | 48 capsules | 26 capsules per | 13 capsules per bottle |

Package Storage and Handling. Mocetinostat drug product is packaged in high-density polyethylene (HDPE), white opaque bottles. The 10 mg and 25 mg capsules are packaged into 48 count 75 cc bottles. The 20 mg capsules are packaged into 26 count 60 cc bottles. The 50 mg capsules are packaged into 13 count 60 cc bottles. A tamper-proof heat induction seal and a child-resistant closure are used for all dosage strengths. Each bottle is labeled with contents, product lot number, required storage conditions, expiration date, sponsor address and regional specific cautionary statement “New Drug-Limited by Federal Law to Investigational Use.”

Drug product storage conditions should reflect label instructions. Mocetinostat drug product 10 mg and 25 mg capsules are labeled for storage at refrigerated conditions between 2° C. and 8° C. (36° F. and 46° F.) or room temperature conditions between 15° C. and 30° C. (59° F. and 86° F.) depending on the drug product lot. All Mocetinostat drug product 20 mg and 50 mg capsules will be labeled for storage at room temperature conditions between 15° C. and 30° C. (59° F. and 86° F.).

Drug will be stored in investigational pharmacy.

### Administration Guidelines

### Mocetinostat Will be Administered Orally.

### Vinorelbine

Description Vinorelbine is a semi-synthetic vinca alkaloid which acts via inhibition of mitotic microtubule formation.

Dosage form, strength and route of administration. Vinorelbine is formulated as an injectable solution containing an equivalent of 10 mg (1 ml vial or 50 mg (5 ml vial) in sterile water. The appropriate dose of vinorelbine to be administered to each subject will be calculated at each treatment cycle based on the subject's BSA.

Supply and stability information and storage conditions. Unopened vials of Vinorelbine are stable at temperatures up to 25° C. (77° F.) for up to 72 hours or until the date indicated on the package when stored under refrigeration at 2-8° C. (36-46° F.) and protected from light in the carton. Diluted Vinorelbine may be used for up to 24 hours under normal room light when stored in polypropylene syringes or polyvinyl chloride bags at 5-30° C. (41-85° F.).

Refer to the FDA package insert for further information. Commercial drug will be used.

Administration guidelines. Vinorelbine injection must be diluted in either a syringe or I.V. bag. The calculated dose of Vinorelbine should be diluted according to the pharmacy manual. The diluted Vinorelbine should be administered intravenously over 6 to 10 minutes into the side port of a free-flowing I.V. followed by flushing with at least 75 to 125 mL of one of the solutions. Refer to the FDA package insert for further information.

Procedures for monitoring subject compliance. Calculations of the dose administered will be documented in the source documents at the site and confirmed during monitoring visits by the clinical monitor. In addition, the dose administered will be documented in the electronic CRFs. Any interruptions or discontinuations of the medications will be documented in both the source documents and the electronic CRFs.

Accountability. Master drug accountability forms will be used to maintain accurate records of receipt, distribution, and return of all drug supplies shipped to the site from the sponsor's representative. A drug accountability form will be maintained at each location where drug is stored for subject administration, i.e., main pharmacy, satellite pharmacy, physician's office or other dispensing areas. Individual subject drug accountability forms will be used to maintain accurate records of drug dispensed, returned and consumed by each subject. When the clinical site receives supplies of mocetinostat and the custom diluent from the sponsor, or its representative, a visual inspection of the drug will be conducted and the condition of supplies will be recorded. Any damaged or missing supplies are to be reported on the accountability forms. The date, investigational drug lot numbers, and the amount of drug received will be documented on a master drug accountability form. During the course of the study, the initials and number of each subject to whom drug is dispensed, the date, quantity of drug dispensed, all transfers, returns, and disposal/destruction of drug are to be documented on the accountability forms. Drug supplies will be stored in a secure, limited-access storage area under the recommended storage conditions. Regular periodic inventory of the investigational drug supply will be performed.

Disposal. Previously dispensed drug returned to the study pharmacy should be collected and stored separately from undispensed drug. The return or disposal of previously dispensed drug will be authorized only after accountability has been verified by the study monitor, unless the site's SOPs require used drug to be disposed of immediately. The study site will be allowed to dispose previously dispensed drug only if a record of destruction can be provided. Study site personnel will return, dispose or transfer all drug as directed by the sponsor or its representative. It is the responsibility of the investigator or representative to maintain investigational drug accountability, complete return/disposal/transfer records and ensure that the unused study drug is appropriately returned, disposed of or transferred. The return/disposal/transfer records should be signed by the investigator or representative and by a witness. If undispensed study drug is returned or transferred, a copy of the return/transfer records is to be included in the shipment. If any study drug cannot be accounted for, a written explanation on official stationary signed by the investigator must be included with the drug accountability records.

### Ethical Considerations

Institutional Review Board (IRB)/Independent Ethics Committee (IEC). The IRB/IEC is responsible for the review and approval of relevant study documentation to assure the protection of the rights and welfare of human subjects. Relevant documents requiring review and approval by an IRB/IEC include but are not limited to; the study protocol and amendments, the subject written informed consent form and consent form updates, subject recruitment documentation (e.g., advertisements), written information provided to subjects, Investigator's Brochure (IB) and any revisions or Addenda, available safety information, subjects payment/compensation, investigator's current curriculum vitae (or other qualification documentation), and any other documents that the IRB/IEC may need to fulfill its responsibilities. The IRB/IEC is required to operate in compliance with current regulations of the local regulatory authorities, the International Conference on Harmonisation (ICH) guidelines and current Good Clinical Practice (cGCP) guidelines. Written approval from the IEC must be obtained before the study can be started (consent of the first subject) or before the investigational study drug is administered to a subject.

Changes to the study requiring an amendment to the protocol or changes to the informed consent form; must be approved in writing by the IRB/IEC. IRB/IEC approval must be obtained prior to the implementation of such changes.

The sponsor or representative will report promptly to the investigator any new information that may indicate an adverse effect on the safety of the subjects or the conduct of the study. The investigator is responsible for informing the IRB/IEC of any new safety information (e.g., safety report presented as an IB Addendum), and for reporting the progress of the study. At the end of the study, defined as the last visit of the last subject, the investigator will provide a final report to the IRB/IEC (if required).

Ethical Conduct of the Study. The clinical study will be conducted in accordance with the ethical principles that have their origin in the Declaration of Helsinki (in its revised edition, Tokyo 2004), and that are consistent with the guidelines for current Good Clinical Practice (cGCP) and applicable regulatory requirements.

Informed Consent. The principal investigator is responsible for ensuring that no subject undergoes any study-related examination or activity before that subject has given written informed consent to participate in the study. The subject must give the written consent only after detailed information about the study has been provided. The verbal explanation will cover all the elements specified in the written information provided to the subject.

An investigator or representative will inform the subject of the aims, methods and potential hazards of the study. The subject must be given every opportunity to clarify any points he/she does not understand and, if necessary, ask for more information. At the end of the informed consent discussion, the subject will be given time to consider the study information and to freely decide his/her participation. If the subject agrees to participate in the study, the informed consent document must be signed by both the subject t and by the person who conducted the informed consent discussion. A copy of the signed consent will be given to the subject and the original will be archived in the investigator site file.

It should be emphasized to the subject that he/she is free to withdraw from the study at any time. Subjects who refuse to give or who withdraw written informed consent should not be included or continue in the study.

Confidentiality of records. The investigator must assure that the subjects' anonymity will be maintained. Subjects should not be identified by name on any documents submitted to the sponsor or during verbal communications.

Subjects will be identified with their initials and a protocol-assigned subject number.

The investigator will maintain all signed informed consent forms in strict confidence, and will maintain a separate log of subjects' initials and hospital/clinic accession number.

All laboratory specimens and evaluation forms will be identified using only a coded number, subject number, subject initials and/or date of birth in order to maintain confidentiality. All records will be kept in a secured area in the clinical research unit. Computer entry and networking programs will be performed using coded numbers.

The subject will be informed that all clinical information is confidential, and must consent to direct access to his/her original medical records and study data for study related sponsor monitoring, audit, IRB/IEC review and regulatory inspection.

Monitoring of the Study and Regulatory Compliance. The UCLA data safety monitoring staff with serve as the data safety monitoring board for this study.

Site visits. Monitoring visits will be conducted by representatives of the Sponsor according to the U.S. CFR Title 21 Parts 50, 56, and 312 and ICH Guidelines for GCP (E6). By signing this protocol, the Investigator grants permission to the Sponsor (or designee), and appropriate regulatory authorities to conduct on-site monitoring and/or auditing of all appropriate study documentation. Regular monitoring Case report form entries will be verified with source documentation at regular monitoring visits.

### Case Report Forms (CRFs)

CRF completion. The Investigator will prepare and maintain adequate and accurate source documents designed to record all observations and other pertinent data for each subject treated with the study drug. Study personnel at each site will enter data from source documents corresponding to a subject's visit into the protocol-specific electronic Case Report Form (eCRF) OR paper CRF when the information corresponding to that visit is available. Subjects will not be identified by name in the study database or on any study documents to be collected by the Sponsor (or designee), but will be identified by a site number, subject number and initials.

For eCRFs: If a correction is required for an eCRF, the time and date stamps track the person entering or updating eCRF data and creates an electronic audit trail. For paper CRFs: If a correction is made on a CRF, the study staff member will line through the incorrect data, write in the correct data and initial and date the change.

The Investigator is responsible for all information collected on subjects enrolled in this study. All data collected during the course of this study must be reviewed and verified for completeness and accuracy by the Investigator. A copy of the CRF will remain at the Investigator's site at the completion of the study.

CRF correction. Any modification of previously entered CRF data must be made by following the procedures in place for the completion of electronic CRFs.

Data management. The data will be entered into a validated database. The Data Management group will be responsible for data processing, in accordance with procedural documentation. Database lock will occur once quality assurance procedures have been completed. All procedures for the handling and analysis of data will be conducted using good computing practices meeting FDA guidelines for the handling and analysis of data for clinical trials.

After data have been entered into the study database, a system of computerized data validation checks will be implemented and applied to the database on a regular basis. Query reports (Data Clarification Requests) pertaining to data omissions and discrepancies will be forwarded to the Investigators and study monitors for resolution. The study database will be updated in accordance with the resolved queries. All changes to the study database will be documented.

Source data and documentation. Source data is all information in original records and copies of original records of clinical findings, observations, or other activities in a clinical study necessary for the reconstruction and evaluation of the study. Source data are contained in source documents. Source documents are original documents, data and records, and include but are not limited to; hospital records, clinical/office charts, laboratory notes, memoranda, subject diaries/checklists, pharmacy dispensing records, recorded data from automated instruments, copies/transcriptions certified after verification as being accurate, microfiches, photographic negatives, microfilm or magnetic media, x-rays, subject files, pharmacy records, laboratory records, and other medico-technical records.

All source documents produced in this study will be maintained by the investigator and made available for inspection by representatives of the sponsor and/or regulatory authorities. The original signed informed consent form for each participating subject will be retained by the investigator and a copy given to the subject.

The database is safeguarded against unauthorized access by established security procedures; appropriate backup copies of the database and related software files will be maintained. Databases are backed up by the database administrator in conjunction with any updates or changes to the database.

Investigator study files. The principal investigator is responsible for maintaining all study related documents in study files. The sponsor will notify investigators when retention of study files is no longer necessary. The following documents will be kept in the study files or be readily accessible. This list is not comprehensive and additional documents may be required for this study. Your assigned clinical monitor will specify the required documents:
the original protocol and all amendments
a signed agreement or protocol “Investigator's Statement”
a signed and dated study staff roles and responsibilities log
a copy of the current curriculum vitae of the principal investigator and of all subinvestigators
an IRB/IEC membership list and all IRB/IEC approvals for the protocol and amendments, informed consent documentation and all updates, advertisements, and written information provided to subjects; all IRB/IEC correspondence; documentation that the IB, IB Addenda and subsequent revisions have been submitted to the IRB/IEC and regulatory authorities (as applicable); documentation that all unexpected SAEs and any periodic safety reports have been submitted to the IRB/IEC; and annual IRB/IEC reports (as required)
regulatory authority approval/notifications (as required)
safety reports/notifications sent from the sponsor
an updated laboratory certification and the laboratory's normal values (covering entire time interval of study for all laboratory tests conducted during the study)
record of retained biological samples
all confirmations of investigational drug receipt, shipping records, drug accountability logs, and drug return/disposal records
sample investigational drug label (as required)
initiation visit report
insurance certificate (where required)
randomization list and decoding procedures (if applicable)
final completed CRFs for all subjects
CRF query/resolution records
all correspondence to/from the sponsor or its representatives
a blank informed consent form and a blank CRF
the Investigator's Brochure or similar compound-specific background document
IB Addenda (if applicable)
a subject screening log
an unambiguous subject enrollment log (e.g., contains subject initials, protocol accession number, clinic or hospital number)
all subjects' signed and dated informed consents
a site visit log

Data protection. When personal data on subjects are stored or processed electronically, the data must be protected to prevent their disclosure to unauthorized third parties. In the USA, the investigator is required to follow the privacy regulations for the use or disclosure of subject health information as set forth in the Privacy Rule (entitled the “Standards for Privacy of Individually Identifiable Health Information”) under the Health Insurance Portability and Accountability Act of 1996 (HIPAA).

Discontinuation of study. The Sponsor reserves the right to discontinue the study at any time for clinical or administrative reasons. Such a termination must be implemented by the investigator, if instructed to do so by the Sponsor, in a time frame that is compatible with the wellbeing of study subjects.

Retention of records. Essential study documents (i.e., CRFs, source documents, study regulatory files) will be retained according to ICH and GCP guidelines until at least 2 years after the last approval of a marketing application and until there are no pending or contemplated marketing applications or at least 2 years have elapsed since the formal discontinuation of clinical development of the investigational product. These documents should be retained for a longer period, however, if required by the applicable regulatory requirements or by an agreement with the sponsor. The sponsor will inform the investigator/institution in writing when the trial-related records are no longer needed.

Publication of data and protection of intellectual property. The investigator(s) agrees to inform the central PIs and sponsor of any proposed publication(s) or presentation(s) on the study. The central PIs (Dr. Federman and Dr. Singh) will establish the authorship and authorship priority in collaboration with the sponsor. All proposed publications, abstracts or presentations (in outline form with copies of slides) will be submitted to the sponsor at least 30 days prior to the submission of the data for publication to allow the sponsor to protect its proprietary information. The sponsor will review the submitted material within a reasonable period of time and will not unreasonably withhold publication permission.

Compliance statement. The study will be conducted in accordance with standards that meet regulations relating to current Good Clinical Practice. These standards respect the following guidelines: current Good Clinical Practice; Consolidated Guideline (International Conference on Harmonization of Technical Requirements for the Registration of Pharmaceuticals for Human Use, May 1996); United States (US) Code of Federal Regulations (CFR) dealing with clinical studies (21 CFR parts 50, 54, 56, 312, and 314), and the Declaration of Helsinki.

Quality control. The sponsor, or its representative, will be responsible for implementing and maintaining quality assurance and quality control systems with written SOPs to ensure that the study is conducted and data are generated, documented (recorded), and reported in compliance with the protocol, cGCP and the applicable regulatory requirements.

Quality assurance. Quality Assurance (QA) audits may be carried out on critical phases during the clinical and reporting phases of the study. Phases selected for audit may include (but will not be limited to): dose preparation, dosing and protocol compliance, CRF data review, special assay inspection, database, data listings and tables, draft clinical report and final clinical report. If an audit is conducted, audits will be carried out by a qualified quality assurance representative, independent of the staff involved in the study. Records of these audits will be documented and distributed to the sponsor for review.

Statistical Considerations. The clinical outcomes, laboratory, PK, and other safety data from both segments of the study will be analyzed descriptively. In addition to determining DLT and RP2D, results will be analyzed to determine if a sufficient response signal and safety profile justifies further study. Descriptive statistical summaries for demographic and subject baseline characteristics will be produced, as well as statistical summaries of safety, efficacy and pharmacokinetic/pharmacodynamic results, where categories for statistical summaries will consist of the dose level initially assigned for Phase 1 dose escalation, and the initial dose in Phase 2 (RP2D). In addition, exploratory analyses of both toxicity, response (ORR) and pharmacodynamic data will be performed for both the assigned and actual daily doses of drugs, the actual number of days of treatment, and for cumulative exposure to study drug as expressed by the product sum of dose over time (area under the dose-time curve).

Sample size. The sample size for the dose-escalation phase of the study will be determined by the number of escalation steps and the required sample within cohort (3 or 6 subjects). The sample size for the expansion phase will be 20 subjects.

Safety. All subjects who receive any amount of study drug will be included in the safety analyses. All adverse events will be mapped to preferred terms and system organ classes using the MedDRA dictionary.

Subject incidence of adverse events will be displayed by dose group and by system organ class. Adverse events will also be summarized by severity and relationship to study drug. Subject incidence of serious adverse events will also be summarized. The type and number of DLTs will be separately presented by dose group, as appropriate. Laboratory parameters will be summarized using descriptive statistics at baseline and at each post-baseline time point. Changes from baseline will also be summarized.

Pharmacokinetics. Pharmacokinetic parameter values will be summarized by descriptive statistics at each dose level.

Pharmacodynamics. Pharmacodynamic variables will be summarized by dose group and time point. Correlations between pharmacodynamic variables and efficacy variables may also be performed.

Efficacy. A modified intent to treat (mITT) approach will be used for efficacy analysis, in which the mITT population will consist of all subjects who receive at least a partial dose of study therapy. Tumor response rates will be summarized by dose group and for all subjects who receive the RP2D, including those from the dose escalation and expansion phases. Responses will be classified as CR, PR, SD or PD according to RECISTv.1.1 criteria. Summaries will be based on the best response recorded up until disease progression. Subjects who discontinue prior to the first 8-weekly response assessment will be considered as non-responders in the primary efficacy analysis. Objective tumor response (CR or PR) will also be summarized, as will PFS, OS, DCR and duration of response and DCR. Time to event data will be summarized by Kaplan-Meier methods, including 25th, 50th (median) and 75th percentiles with point estimates and two-sided 95% confidence intervals, as well as number and percent of censored observations.

Statistical analysis. The statistical analyses will be reported using summary tables, figures, and data listings. Continuous variables will be summarized with means, standard deviations, medians, minimums, and maximums. Categorical variables will be summarized by counts and by percentage of subjects in corresponding categories. All raw data obtained from the case report forms as well as any derived data will be included in data listings. All analyses will be based on the Safety Population, which will include all subjects who receive any amount of study drug.

11.1 Safety analysis. All subjects who receive any amount of study drug will be included in the safety analyses. All adverse events will be mapped to preferred terms and system organ classes using the MedDRA dictionary.

Subject incidence of adverse events will be displayed by dose group and by system organ class. Adverse events will also be summarized by severity and relationship to study drug. Subject incidence of serious adverse events will also be summarized. Laboratory parameters will be summarized using descriptive statistics at baseline and at each post-baseline time point. Changes from baseline will also be summarized. In addition, shift tables (i.e., CTCAE grade at baseline versus CTCAE grade at follow-up) will be provided to assess changes in laboratory values from baseline to follow-up.

### Study Management

### Compliance

Compliance with the Protocol and Protocol Revisions. The study shall be conducted as described in this approved protocol. All revisions to the protocol must be discussed with Mirati. The investigator should not implement any deviation or change to the protocol without prior review and documented approval/favorable opinion from the IRB of an amendment, except where necessary to eliminate an immediate hazard(s) to study subjects.

If a deviation or change to a protocol is implemented to eliminate an immediate hazard(s) prior to obtaining IRB/IEC approval/favorable opinion, as soon as possible the deviation or change will be submitted to:
IRB/IEC for review and approval/favorable opinion
Regulatory Authority(ies), if required by local regulations
Documentation of approval signed by the chairperson or designee of the IRB(s)/IEC(s) must be sent to Mirati.

If an amendment substantially alters the study design or increases the potential risk to the subject: (1) the consent form must be revised and submitted to the IRB(s)/IEC(s) for review and approval/favorable opinion; (2) the revised form must be used to obtain consent from subjects currently enrolled in the study if they are affected by the amendment; and (3) the new form must be used to obtain consent from new subjects prior to enrollment. If the revision is done via an administrative letter, investigators must inform their IRB(s)/IEC(s).

Monitoring. The sponsor and UCLA representatives will review data centrally to identify potential issues to determine a schedule of on-site or teleconference visits for targeted review of study records.

Certain CRF pages and/or electronic files may serve as the source documents:

In addition, the study may be evaluated by UCLA internal auditors and government inspectors who must be allowed access to CRFs, source documents, other study files, and study facilities. UCLA audit reports will be kept confidential.

The investigator must notify Mirati promptly of any inspections scheduled by regulatory authorities, and promptly forward copies of inspection reports to Mirati.

12.1.2.1 Source Documentation. The Investigator is responsible for ensuring that the source data are accurate, legible, contemporaneous, original and attributable, whether the data are hand-written on paper or entered electronically. If source data are created (first entered), modified, maintained, archived, retrieved, or transmitted electronically via computerized systems (and/or any other kind of electronic devices) as part of regulated clinical trial activities, such systems must be compliant with all applicable laws and regulations governing use of electronic records and/or electronic signatures. Such systems may include, but are not limited to, electronic medical/health records (EMRs/EHRs), adverse event tracking/reporting, protocol required assessments, and/or drug accountability records).

When paper records from such systems are used in place of electronic format to perform regulated activities, such paper records should be certified copies. A certified copy consists of a copy of original information that has been verified, as indicated by a dated signature, as an exact copy having all of the same attributes and information as the original.

### 12.2. Records

12.2. 1 Records Retention. The investigator must retain all study records and source documents for the maximum period required by applicable regulations and guidelines, or institution procedures, or for the period specified by UCLA. The investigator must contact Mirati prior to destroying any records associated with the study.

Mirati will notify the investigator when the study records are no longer needed.

If the investigator withdraws from the study (eg, relocation, retirement), the records shall be transferred to a mutually agreed upon designee (eg, another investigator, IRB).

12.2.2 Study Drug Records. It is the responsibility of the investigator to ensure that a current disposition record of study drug (inventoried and dispensed) is maintained at the study site to include investigational product and the following non-investigational product(s). Records or logs must comply with applicable regulations and guidelines and should include:
amount received and placed in storage area
amount currently in storage area
label identification number or batch number
amount dispensed to and returned by each subject, including unique subject identifiers
amount transferred to another area/site for dispensing or storage
non-study disposition (e.g., lost, wasted)
amount destroyed at study site, if applicable
retain samples for bioavailability/bioequivalence, if applicable
dates and initials of person responsible for Investigational Product dispensing/accountability, as per the Delegation of Authority Form.

12.2.3. Case Report Forms. Data that are derived from source documents and reported on the CRF must be consistent with the source documents or the discrepancies must be explained. Additional clinical information may be collected and analyzed in an effort to enhance understanding of product safety. CRFs may be requested for AEs and/or laboratory abnormalities that are reported or identified during the course of the study.

The confidentiality of records that could identify subjects must be protected, respecting the privacy and confidentiality rules in accordance with the applicable regulatory requirement(s).

The investigator will maintain a signature sheet to document signatures and initials of all persons authorized to make entries and/or corrections on CRFs.

The completed CRF, SAE/pregnancy CRFs, must be promptly reviewed, signed, and dated by the investigator or qualified physician who is a subinvestigator and who is delegated this task on the Delegation of Authority Form. The investigator must retain a copy of the CRFs including records of the changes and corrections.

12.3. Clinical Study Report and Publications. A Signatory Investigator must be selected to sign the clinical study report.

The data collected during this study are confidential and proprietary to UCLA. Any publications or abstracts arising from this study must be cleared by the central PIs (Dr. Federman and Dr. Singh) and must adhere to the publication requirements set forth in the clinical trial agreement (CTA) governing participation in the study.

### REFERENCES

1. Batlevi CL, Crump M, Andreadis C, Rizzieri D, Assouline S E, Fox S, van der Jagt R H C, Copeland A, Potvin D, Chao R et al. A phase 2 study of mocetinostat, a histone deacetylase inhibitor, in relapsed or refractory lymphoma. Br J Haematol. 2017; 178 (4): 434-441.
2. Blum K A, Advani A, Fernandez L, Van Der Jagt R, Brandwein J, Kambhampati S, Kassis J, Davis M, Bonfils C, Dubay M et al. Phase II study of the histone deacetylase inhibitor MGCD0103 in patients with previously treated chronic lymphocytic leukaemia. Br J Haematol. 2009; 147 (4): 507-514.
3. Bonfils C, Kalita A, Dubay M, Siu L L, Carducci M A, Reid G et al. Evaluation of the pharmacodynamic effects of MGCD0103 from preclinical models to human using a novel HDAC enzyme assay. Clin Cancer Res. 2008; 14:3441-3449.
4. Boumber Y, Younes A & Garcia-Manero G, Mocctinostat (MGCD0103): a review of an isotype-specific histone deacetylase inhibitor. Expert Opinion on Investigational Drugs. 2011; 20 (6) 823-829.
5. Casanova M, Ferrari A, Spreafico F, Terenziani M, Massimino M, Luksch R, Cefalo G, Polastri D, Marcon I, Bellani F F. Vinorelbine in previously treated advanced childhood sarcomas: evidence of activity in rhabdomyosarcoma. Cancer. 2002;94 (12): 3263-8.
6. Casanova M, Ferrari A, Bisogno G, Merks J H, De Salvo G L, Meazza C, Tettoni K, Provenzi M, Mazzarino I, Carli M. Vinorelbine and low-dose cyclophosphamide in the treatment of pediatric sarcomas: pilot study for the upcoming European Rhabdomyosarcoma Protocol. Cancer. 2004; 101 (7): 1664-71.
7. Chan E, Chiorcan E G, O'Dwyer P J, Gabrail N Y, Alcindor T, Potvin D, Chao R, Hurwitz H. Phase I/II study of mocetinostat in combination with gemcitabine for patients with advanced pancreatic cancer and other advanced solid tumors. Cancer Chemother Pharmacol. 2018;81 (2): 355-364.
8. Dantonello T M, Int-Veen C, Winkler P, et al. Initial patient characteristics can predict pattern and risk of relapse in localized rhabdomyosarcoma. J Clin Oncol. 2008; 26:406.
9. Fournel M, Bonfils C, Hou Y, Yan P T, Trachy-Bourget M C, Kalita A, Liu J, Lu A H, Zhou N Z, Robert M F et al. MGCD0103 a novel isotype-selective histone deacetylase inhibitor, has broad spectrum antitumor activity in vitro and in vivo. Mol Cancer Ther. 2008: 7 (4): 759-768.
10. Garcia-Manero G, Assouline S, Cortes J, Estrov Z, Kantarjian H, H, Newsome W M, Miller W H Jr, Rousseau C, Kalita A et al. Phase 1 study of the oral isotype specific histone deacetylase inhibitor MGCD0103 in leukemia. Blood. 2008; 112 (4): 981-989.
11. Gavrilov V, Lavrenkov K, Ariad S, Shany S. Sodium valproate, a histone deacetylase inhibitor, enhances, the efficacy of vinorelbine-cisplatin-based chemoradiation in non-small cell lung cancer cells. Anticancer Res. 2014;34 (11): 6565-72.
12. Glozack M A & Seto E. Histone dacetylases and cancer. Oncogene. 2007; 26:5420-5432.
13. Hedrick E, Crose L, Linardic C M, Safe S. Histone Deacetylase Inhibitors Inhibit Rhabdomyosarcoma by Reactive Oxygen Species-Dependent Targeting of Specificity Protein Transcription Factors. Mol Cancer Ther. 2015; 14 (9): 2143-53.
14. Howlader N, Noone A M, Krapcho M, Miller D, Bishop K, Kosary CL, Yu M, Ruhl J, Tatalovich Z, Mariotto A, Lewis D R, Chen H S, Feuer E J, Cronin K A (eds). SEER Cancer Statistics Review, 1975-2014, National Cancer Institute. Bethesda, MD, https://seer.cancer.gov/csr/1975_2014/, based on November 2016 SEER data submission, posted to the SEER web site, April 2017.
15. Keshclava N, Houghton P J, Morton C L, Lock R B, Carol H, Keir S T, et al. Initial testing (stage 1) of vorinostat (SAHA) by the pediatric preclinical testing program. Pediatr Blood Cancer. 2009; 53 (3): 505-8.
16. Kim M S, Kwon H J, Lee Y M, Back J H, Jang J E, Lee S W et al. Histone deacetylases induce angiogenesis by negative regulation of tumor suppressor genes. Nat Med. 2001; 7:437-443.
17. Kuttesch J R Jr, Krailo M D, Madden T, Johansen M, Bleyer A, Children's Oncology Group. Phase II evaluation of intravenous vinorelbine (Navelbine) in recurrent or refractory pediatric malignancies: a Children's Oncology Group Study. Pedir Blood Cancer. 2009; 53 (4): 590-3.
18. Marks P, Rifkind R A, Richon V M, Breslow R, Miller T, Kelly W K. Histone deacetylases and cancer: causes and therapies. Nat Rev Cancer. 2001; 1 (3): 194-202.
19. Minard-Colin V, Ichante J L, Nguygen L, Paci A, Orbach D, Bergeron C, Defachelles A S, Andre N, Corradini N, Schmitt C, Tabone M D, Blouin P, Sirvent N, Goma G, Geoerger B, Oberlin O. Phase II study of vinorelbine and continuous low doses cyclophosphamide in children and young adults with a relapsed or refractory malignant solid tumor: good tolerance profile and efficacy in rhabdomyosarcoma—a report from the Societe Francaise de l'enfant et de l'adolescent (SFCE). Eur J Cancer. 2012; 48 (15): 2409-16.
20. Ognjanovic S, Linabery A M, Charbonneau B, Ross J A. Trends in childhood rhabdomyosarcoma incidence and survival in the United States, 1975-2005. Cancer. 2009; 115 (18): 4218-26 . . .
21. Pappo A S, Anderson J R, Crist W M, et al. Survival after relapse in children and adolescents with rhabdomyosarcoma: A report from the Intergroup Rhabdomyosarcoma Study Group. J Clin Oncol. 1999; 17:3487.
22. PDQ Pediatric Treatment Editorial Board. Childhood Rhabdomyosarcoma Treatment (PDQ®): Health Professional Version. 2018 Feb. 2. In: PDQ Cancer Information Summaries [Internet]. Bethesda (MD): National Cancer Institute (US); 2002-. Available from: https://www.ncbi.nlm.nih.gov/books/NBK65802
23. Punyko J A, Mertens A C, Baker K S, et al. Long-term survival probabilities for childhood rhabdomyosarcoma. A population-based evaluation. Cancer. 2005; 103:1475.
24. Ries L A G, Harkins D, Krapcho M, et al. SEER Cancer Statistics Review, 1975-2003, National Cancer Institute. Bethesda, MD. http://seer.cancer.gov/csr/1975_2003 (Accessed on Jun. 10, 2011).
25. Siu LL, Pili R, Duran I, Messersmith W A, Chen E X, Sullivan R, MacLean M, King S, Brown S, Reid G K et al. Phase I study of MGCD0103 given as a three-times-per-week oral dose in patients with advanced solid tumors. J Clin Oncol. 2008; 26 (12): 1940-1947.
26. Sultan I, Qaddoumi I, Yaser S, et al. Comparing adult and pediatric rhabdomyosarcoma in the surveillance, epidemiology and end results program, 1973 to 2005: an analysis of 2,600 patients. J Clin Oncol. 2009; 27:3391.
27. US Food & Drug Administration. Drug Development and Drug Interactions: Table of Substrates, Inhibitors and Inducers. Available at: FDA.gov website.
28. Winter S, Fasola S, Brisse H, et al. Relapse after localized rhabdomyosarcoma: Evaluation of the efficacy of second-line chemotherapy. Pediatr Blood Cancer. 2015; 62:1935.
29. Vleeshouwer-Neumann T, Phelps M, Bammler T K, MacDonald J W, Jenkins I, Chen E Y. Histone deacetylase inhibitors antagonize distinct pathways to suppress tumorigenesis of embryonal rhabdomyosarcoma. PLOS One. 2015; 10 (12): c0144320
30. Younes A, Oki Y, Bocick R G, Kuruvilla J, Fanale M, Neclapu S, Copeland A, Buglio D, Galal A, Besterman J et al. Mocetinostat for relapsed classical Hodgkin's lymphoma: an open-label, single-arm, phase 2 trial. Lancet Oncol. 2011; 12 (13): 1222-1228
31. Zhang Q, Sun M, Zhou S and Guo B. Class I HDAC inhibitor mocetinostat induces apoptosis by activation of miR-31 expression and suppression of E2F6. Cell Death Disc. 2016; 2:16036.
32. Zhou N, Moradei O, Raeppel S, Leit S, Frechette S, Gaudette F, Paquin I, Bernstein N, Bouchain G, Vaisburg A et al. Discovery of N-(2-aminophenyl)-4-[(4-pyridin-3-ylpyrimidin-2-ylamino)methyl]benzamide (MGCD0103), an orally active histone deacetylase inhibitor. J Med Chem. 2008; 51 (14): 4072-5.

### Example 2

### Mocetinostat with Vinorelbine in Children, Adolescents & Young Adults with Refractory and/or Recurrent Rhabdomyosarcoma

This phase I trial studies the side effects and best dose of mocetinostat when given together with vinorelbine to see how well it works in treating children, adolescents, and young adults with rhabdomyosarcoma that has spread to nearby tissues or lymph nodes and cannot be removed by surgery (locally advanced unresectable) or has spread to other places in the body (metastatic), and does not respond to treatment (refractory) or has come back (relapsed).

Mocetinostat may stop the growth of tumor cells by blocking some of the enzymes needed for cell growth. Drugs used in chemotherapy, such as vinorelbine, work in different ways to stop the growth of tumor cells, either by killing the cells, by stopping them from dividing, or by stopping them from spreading. Giving mocetinostat and vinorelbine may work better in treating children, adolescents, and young adults with rhabdomyosarcoma compared to vinorelbine alone.

### Primary Objectives:

I. To determine the first cycle dose-limiting toxicities (DLTs), maximum tolerated dose (MTD), and a biologically effective and recommended phase 2 dose (RP2D) of mocetinostat administered orally three times per week for a total of 9 doses per 21 day cycle given in combination with vinorelbine on days 1, 8, 15 of 21 day cycles in subjects with refractory or recurrent rhabdomyosarcoma (RMS). (Phase 1 Dose Escalation)

II. To determine the progression-free survival (PFS), defined as time from first dose of vinorelbine to tumor progression or death due to any cause, at the RP2D of mocetinostat administered orally three times per week starting on day 3 for a total of 9 doses per 21 day cycle given in combination with vinorelbine on days 1, 8, 15 of a 21 day cycle in subjects with refractory or recurrent RMS. (Expansion Cohort).

### Secondary Objectives:

I. Safety profile of mocetinostat in combination with vinorelbine as characterized by adverse event (AE) type, severity, timing and relationship to study drugs, as well as laboratory abnormalities in the first and subsequent treatment cycles. (Phase 1 Dose Escalation)

II. Pharmacokinetics (PK) of mocetinostat in plasma. (Phase 1 Dose Escalation)

$$III . Clinical benefit rate ( CBR = complete response [ CR ] + partial response [ PR ] and stable disease [ SD ] ) of mocetinostat + vinorelbine in metastatic / refractory / unresectable RMS . ( Phase 1 Dose Escalation )$$

IV. Antitumor activity of mocetinostat+vinorelbine in refractory/recurrent RMS as measured by overall response rate (ORR), duration of response (DOR), disease control (DC), duration of disease control, as well as progression-free survival (PFS). (Phase 1 Dose Escalation)

V. Pharmacodynamics of mocetinostat on molecular targets in surrogate tissue. (Phase 1 Dose Escalation and Expansion Cohort)

VI. Exploratory biomarker development to enable prediction of drug toxicity, tumor response and the mechanism(s) of acquired study drug resistance. (Phase 1 Dose Escalation and Expansion Cohort)

VII. Obtain RMS tissue biological samples pre-treatment and at progression to assess for differences in gene expression by next gen (generation) sequencing and ribonucleic acid (RNA) sequencing (seq). (Phase 1 Dose Escalation and Expansion Cohort)

VIII. Antitumor activity of mocetinostat+vinorelbine in metastatic/refractory RMS as measured by overall response rate (ORR) and duration of response (DOR), disease control (DC), duration of disease control, as well as progression-free survival (PFS) according to Response Evaluation Criteria in Solid Tumors (RECIST) version (v) 1.1. (Expansion Cohort)

IX. Safety and tolerability of mocetinostat and vinorelbine as characterized by adverse event type, severity, timing and relationship to study drug, as well as laboratory abnormalities. (Expansion Cohort)

Study Type: Interventional (Clinical Trial)

Estimated Enrollment: 38 participants

Intervention Model: Single Group Assignment

Masking: None (Open Label)

Primary Purpose: Treatment

Primary Outcome Measures:

1. To describe any dose-limiting toxicity (DLT) [Time Frame: 1 year] Percentage of subjects with dose-limiting toxicities (DLTs) as assessed by NCI CTCAE (Version 4.03)

2. To determine the maximum tolerated dose (MTD) or highest protocol defined doses (in the absence of exceeding the MTD) [Time Frame: 1 year] The MTD is the highest dose associated with first-cycle DLT in <33% of subjects

### Secondary Outcome Measures:

1. To determine the Recommended Phase 2 Dose (RP2D) for mocetinostat in combination with vinorelbine [Time Frame: 1 year] The RP2D may be determined by the MTD or optimal target inhibition with an acceptable safety profile

2. Incidence of Adverse Events (AEs) as assessed by NCI CTCAE (Version 4.03) [Time Frame: 1 year] Assess incidence of all AEs by NCI CTCAE (Version 4.03) grades 1-5

3. Objective Tumor Response [Time Frame: 2 years] Measured using RECIST, Version 1.1

4. Progression Free Survival (PFS) [Time Frame: 2 years] Estimated using Kaplan-Meier methodology

5. Disease Control (DC) [Time Frame: 2 years]

Proportion of subjects with a confirmed Complete Response (CR), Partial Response (PR), or Stable Disease (SD) according to RECIST v1.1

6. Duration of Response (DOR) [Time Frame: 2 years]

Measured from the first date a response is identified (either CR or PR) until the date of disease progression.

7. Area under the Plasma Concentration versus Time Curve (AUC) of mocetinostat [Time Frame: 2 years] Continuous variables will be summarized with means, standard deviations, medians, minimums, and maximums.

8. Clearance (CL) of mocetinostat [Time Frame: 2 years] Continuous variables will be summarized with means, standard deviations, medians, minimums, and maximums.

9. Half-Life [T1/2] of mocetinostat [Time Frame: 2 years] Continuous variables will be summarized with means, standard deviations, medians, minimums, and maximums.

10. Volume of Distribution (Vd) of mocetinostat [Time Frame: 2 years] Continuous variables will be summarized with means, standard deviations, medians, minimums, and maximums.

Ages Eligible for Study: 13 Years and older (Child, Adult, Older Adult)

Sexes Eligible for Study: All

Accepts Healthy Volunteers: No

### Inclusion Criteria:

Willing and able to provide written Institutional Review Board (IRB)/Independent Ethics Committee (IEC)-approved informed consent. For subjects <18 years of age, their parents or legal guardians must sign a written informed consent. Assent, when appropriate, will be obtained according to institutional guidelines.

Have histologically or cytological confirmed diagnosis of rhabdomyosarcoma with locally advanced/unresectable, metastatic, refractory or relapsed disease who have failed standard therapy and for whom no known curative therapy exists.

Measurable disease according to RECIST version 1.1

Prior cancer therapy: Subjects may have received any number of prior therapy regimens. In the investigator's opinion, subjects must have tolerated prior cytotoxic therapies well and have adequate bone marrow reserve. At the time of treatment initiation, at least 3 weeks must have elapsed after prior cytotoxic chemotherapy. At least 7 days must have elapsed since completion of any prior non-cytotoxic cancer therapy and any associated AEs must have resolved.

Prior radiotherapy is allowed if >=2 weeks have elapsed for local palliative radiation therapy (XRT) (small port); >=6 months must have elapsed if prior total body irradiation, craniospinal XRT or if >50% radiation of the pelvis; >6 weeks must have elapsed if other substantial bone marrow radiation (defined per principal investigator's [PI's] discretion). Subjects who have received brain irradiation must have completed whole brain radiotherapy and/or gamma knife at least 4 weeks prior to enrollment.

Subjects with controlled asymptomatic central nervous system (CNS) involvement are allowed in absence of therapy with anticonvulsants. Subjects not requiring steroids or requiring steroids at a stable dose (=<4 mg/day dexamethasone or equivalent) for at least 2 weeks are eligible.

Resolution of all acute toxic effects (excluding alopecia) of any prior anti-cancer therapy to National Cancer Institute (NCI) Common Terminology Criteria for Adverse Events (CTCAE) (version 4.03) grade <1 or to the baseline laboratory values as defined below.

Eastern Cooperative Oncology Group (ECOG) performance status (PS)=<2 in subjects >=17 years old; or Karnofsky/Lansky >50 in subjects <16 years old.

Subjects age >18 years for first cohort. Subjects must be >12 years old for the second and subsequent cohorts. Life expectancy of at least 3 months

Absolute neutrophil count (ANC)>=1000/mm\hat{ }3 (>=1.0×10\hat{ }9/L)

Platelets (PLT)>=100,000/mm\hat{ }3 (>=100×10\hat{ }9/L) (transfusion independent, defined as not receiving platelet transfusions within a 7 day period prior to screening)

Hemoglobin >9.0 g/dL (transfusions are allowed)

Serum creatinine=<1.5×upper limit of normal (ULN) or creatinine clearance >60 mL/min

Total serum bilirubin=<1.5×ULN; =<5×ULN if Gilbert's syndrome

Liver transaminases (aspartate aminotransferase [AST]/alanine aminotransferase [ALT])=<2.5×ULN; =<5×ULN if liver metastases are present

Pregnancy test if female of child-bearing potential negative within 7 days of starting treatment

Cardiac ejection fraction >50% or shortening fraction >28% by echocardiography (ECHO) or multigated acquisition scan (MUGA)

Females of child-bearing potential must have a negative pregnancy test during screening and be neither breastfeeding nor intending to become pregnant during study participation. Females of childbearing potential must agree to avoid pregnancy during the study and commit to abstinence from heterosexual intercourse or agree to use two methods of birth control (one highly effective method and one additional effective method) at least 4 weeks before the start of protocol therapy, for the duration of study participation, and for 6 months after the last dose of mocetinostat.

Males with partner(s) of childbearing potential must take appropriate precautions to avoid fathering a child from the screening period until 90 days after receiving the last dose of mocetinostat. They must commit to abstinence from heterosexual intercourse or agree to use appropriate barrier contraception.

Prior to enrollment of females or males of reproductive potential, the investigator must document confirmation of the subject's understanding of the possible teratogenic effects of mocetinostat.

Willingness and ability to comply with scheduled visits, treatment plan, laboratory tests and other study procedures.

### Exclusion Criteria:

Current participation in another therapeutic clinical trial

### Symptomatic Brain Metastases

History of previous cancer (non RMS), except squamous cell or basal-cell carcinoma of the skin or any in situ carcinoma that has been completely resected, which required therapy within the previous 3 years. Other low grade cancers can be reviewed and allowed at the discretion of the PI.

Incomplete recovery from any surgery (other than central venous catheter or port placement) prior to treatment.

Any of the following in the past 6 months: pericarditis, pericardial effusion, symptomatic congestive heart failure, cerebrovascular accident or transient ischemic attack, pulmonary embolism, deep vein thrombosis, symptomatic bradycardia, requirement for anti-arrhythmic medication.

History of prolonged corrected QT (QTc) interval (e.g., repeated demonstration of a QTc interval >450 milliseconds, unless associated with the use of medications known to prolong the QTc interval). QTc will be calculated using the Bazett formula (RR interval=60/heart rate; QTI corrected=QT interval/sqr [RRinterval]).

History of additional risk factors for torsade de pointes (e.g., heart failure, family history of long QT syndrome)

Use of concomitant medications that increase or possibly increase the risk to prolong the QTc interval and/or induce torsades de pointes ventricular arrhythmia.

Females who are breastfeeding/lactating.

Known active infections (e.g., bacterial, fungal, viral including hepatitis and human immunodeficiency virus [HIV] positivity)

Other severe acute or chronic medical or psychiatric condition or laboratory abnormality that may increase the risk associated with study participation or study drug administration or may interfere with the interpretation of study results and, in the judgment of the investigator, would make the subject inappropriate for entry into this study or compromise protocol objectives in the opinion of the investigator and/or the sponsor.

### Example 3

### Interim Results of a Phase 1 Dose Escalation/Expansion Clinical Trial of Mocetinostat in Combination with Vinorelbine in Adolescents and Young Adults with Refractory and/or Recurrent Rhabdomyosarcoma

Background: Rhabdomyosarcoma (RMS) is the most common soft tissue sarcoma in children and adolescents. About one third of all patients relapse and this setting remains an area of unmet need. Histone deacetylase (HDAC) inhibitors have been shown to have activity in preclinical models of RMS. Mocetinostat (MGCD0103, Mirati Pharmaceuticals, San Diego CA) is an investigational oral (HDAC) inhibitor, that targets HDACs 1, 2, 3 and 11. In an unbiased screening of 600 cell lines spanning all major cancer histologies including 60 sarcoma cell lines, translocation+ and negative RMS cell lines had a higher sensitivity to this agent compared to other cell lines. Mocetinostat displayed high activity in RMS xenograft models and exerted synergistic activity in combination with vinorelbine. Here, we report early interim results of the Phase 1 trial of mocetinostat with vinorelbine in R/R RMS.

Methods: This is an investigator initiated Phase 1 single center, open-label, dose escalation/expansion clinical trial. A modified intent to treat approach is used for efficacy analysis for a target accrual of 20 subjects in the dose expansion cohort. Eligibility criteria include subjects >/=18 years old (yo) for the phase 1 dose escalation cohort and >/=12 years old for the phase 1 dose expansion cohort with a diagnosis of R/R RMS. Mocetinostat 40 mg, 70 mg or 90 mg was taken orally 3 times weekly with vinorelbine 25 mg/m2 IV on day 1,8, and 15 in 21 day cycles. PEG-filgrastim or biosimilar was added if subject experienced grade 3/4 neutropenia in a prior cycle. The maximum tolerated dose of mocetinostat in the dose escalation phase was 40 mg, which was selected as the dose for expansion cohort. Subjects were treated until disease progression by RECIST 1.1 or unacceptable toxicity.

Results: A total of 8 subjects (6 FOXO translocation (+), 1 (−), and 1 unknown) have been enrolled at time of submission. 5 in dose escalation cohort, and 3 in dose expansion. Median age was 19 yo (range 16-63), Median prior treatment regimens were 2 (range 1-4). All patients had measurable metastatic disease. 6 of 8 subjects had prior exposure to vinorelbine in prior salvage chemotherapy or maintenance chemotherapy. As of 20 Jan. 2022 safety cutoff, the most common AEs (all grades) observed in 7 evaluable treated patients regardless of causality include neutropenia (n=5), anemia (n=5), and nausea (n=4). The only grade 3 or 4 treatment related AEs were cytopenias including neutropenia, lymphopenia and anemia. Myelosuppression was transient, reversible and responsive to growth factors. No SAEs related to mocetinostat and/or vinorelbine have been reported. As of efficacy cutoff 20 Jan. 2022, 7 of 8 patients are evaluable for response. 4 subjects had a partial response (PR), 2 subjects had stable disease (SD) and 1 subject had progressive disease for a clinical benefit rate of 86% (CR+PR+SD). Rapid responses were seen in the majority of patients at median of 1.5 months (mos). One of the PR patients progressed at cycle 8 (6 months) and one patient progressed at cycle 3 (2 months). Of the 6 responders, 4 had duration of responses (DOR) ≥6 mo with a median DOR of 8 mos (range 4-16 mo).

Examples of subject data are provided below.

One patient (Subject 4, Cohort 2), a 19 year old female with recurrent, widely metastatic alveolar (FOXO tr+) rhabdomyosarcoma, showed a rapid response at the end of Cycle 2, and PR by RECIST. A significant decrease in standardized uptake value (SUV) on PET. Treatment-related AEs were Gr3 neutropenia. The subject continued on study Cycle 3.

The subject's prior therapy was VAC/Irino, then VAC×44 weeks, Vino/Cyclo×6 months maintenance, Recurrence approx. 6 months from completion of therapy.

Subject 2, Cohort 1, is a 63 year old female with recurrent, refractory alveolar RMS. The subject showed a rapid partial response at the end of Cycle 2 (6 weeks), and Progressive disease (PD) at Cycle 8 (6 months).

Subject 1, Cohort 1, is a 18 year old female with recurrent refractory, metastatic alveolar rhabdomyosarcoma (FOXO transloc+), and a history of chemotherapy and radiation therapy; prior chemotherapy with vincristine, actinomycin, cyclophosphamide (VAC)×46 weeks followed by maintenance 6 months of weekly vinorelbine+cyclophosphamide. Recurrence approx. 6 months after completion of maintenance. In the current study, a rapid response was observed at 6 weeks imaging (end of cycle 2). The subject continues with PR on study Cycle 18. Treatment-related AEs are grade 3/4: anemia, neutropenia.

Subject 03 is an 18 year old male with a history of multiple recurrences, refractory, metastatic alveolar (FOXO transloc+) rhabdomyosarcoma. The subject has SD after Cycle 6 though mostly internal necrosis. Treatment-related AEs are G3/4 neutropenia. The patient decided to discontinue study at Cycle 8 due to difficulty driving. Patient then switched to Vinorelbine/cyclophosphamide (Vino/Cyclo). One month after restarting on Vino/Cyclo the patient progressed with rapid local, regional (axillary lymph nodes) and distant (pulmonary metastases) spread of disease on whole body PET/CT.

Subject 4 is a 20 year old female with a history of recurrent, refractory and metastatic alveolar (Translocation+) rhabdomyosarcoma of the foot who received upfront chemotherapy with VAC+maintenance vinorelbine and cyclophosphamide for 18 months total treatment. She also received an amputation below knee to remove disease in the foot. She recurred with widely metastatic rhabdomyosarcoma 1 year from completion of frontline chemotherapy. She started vinorelbine and mocetinostat with a rapid response and shrinkage of the metastatic disease in widespread lymph nodes at cycle 2 imaging. She remains with partial response at cycle 18.

Subject 5, Cohort 1, is a 28 year old male with a history of metastatic paratesticular embryonal RMS, Transloc (−) unknown, with multiple bone metastases, Prior treatment with VDC×3 cycles with interval progression, VAC 8/20-1/21 Interval progression lung metastases and bone metastases.

The patient showed rapid improvement in bone metastasis pain on Mocetinostat and vinorelbine. Confirmed a PR by RECIST. The patient remains on treatment Cycle 6. No reported SAEs.

Subject 6, Cohort is an 16 yo female with history of recurrent, refractory and metastatic alveolar (FOXO translocation+) rhabdomyosarcoma. Prior treatment was VAC with maintenance vinorelbine and cyclophosphamide for 18 months total. The subject had a rapid response with shrinkage of tumor by cycle 2 and a confirmed radiologic PR after cycle 2 and ongoing PR at cycle 6.

Conclusions: In this interim analysis, Mocetinostat plus vinorelbine shows high efficacy and acceptable safety profile in this heavily pretreated group of refractory relapsed RMS patients. This study is open to accrual and enrollment is ongoing.

What is claimed is:

1. A method of treating a subject having rhabdomyosarcoma (RMS), comprising the steps of administering mocetinostat to the subject, and administering vinorelbine to the subject.

2. The method of claim 1, wherein the subject is a child, an adolescent, or an adult.

3. The method of claim 1, wherein the subject is having a locally advanced RMS, an unresectable RMS, a metastatic RMS, or a recurrent RMS.

4. The method of claim 1 wherein the RMS is alveolar or embryonal.

5. The method of claim 1 wherein the RMS has a FOXO translocation.

6. The method of claim 1 wherein the RMS does not have a FOXO translocation.

7. The method of claim 1, wherein the mocetinostat is administered to the subject prior to, concurrently, or after administering the vinorelbine to the subject.

8. The method of claim 1, wherein the mocetinostat is administered to the subject orally.

9. The method of claim 1, wherein the mocetinostat is administered to the subject at about 40 mg/dose, 70 mg/dose, or 90 mg/dose.

10. The method of claim 1, wherein the mocetinostat is administered to the subject more than one time per week.

11. The method of claim 1, wherein the mocetinostat is administered to the subject three times per week.

12. The method of claim 1, wherein the vinorelbine is administered to the subject intravenously.

13. The method of claim 1, wherein the vinorelbine is administered to the subject weekly.

14. The method of claim 1, wherein the vinorelbine is administered to the subject at a dose of about 25 mg/m².

15. The method of claim 1 wherein the vinorelbine is administered at a dose of 25 mg/m2 IV on day 1, 8, 15 in combination with mocetinostat 40 mg every other day for 9 doses.

16. The method of claim 1 wherein the treatment shows decreased size of the RMS by PET/CT, or the combination thereof.

17. The method of claim 1 wherein treating provides an improvement in Response Evaluation Criteria in Solid Tumors (RECIST version 1.1), improvement in PFS, improvement in Disease Control (DC), improved Duration of Response (DOR), improvement in overall response rate (ORR), or any combination thereof, in the subject or in a population of treated subjects.

18. The method of claim 1 wherein the treating by administering mocetinostat and administering vinorelbine is synergistic.

19. A therapeutic combination for treating rhabdomyosarcoma (RMS), comprising a therapeutically effective amount of mocetinostat and a therapeutically effective amount of vinorelbine.

20. The therapeutic combination of claim 19 which is a synergistic combination.

21. The therapeutic combination of claim 19, wherein the mocetinostat is provided for oral administration.

22. The therapeutic combination of claim 19, wherein the mocetinostat is provided at a dose of about 40 mg/dose, 70 mg/dose, or 90 mg/dose.

23. The therapeutic combination of claim 19, wherein the vinorelbine provided for intravenous administration.

24. The therapeutic combination of claim 19, wherein the vinorelbine provided to administer a dose of about 25 mg/m².

25. The therapeutic combination of claim 19 wherein the vinorelbine is provided for administration at a dose of 25 mg/m2 IV on day 1, 8, 15 in combination with mocetinostat provided for administration at a dose of 40 mg every other day for 9 doses.

26. A method of treating a subject having rhabdomyosarcoma (RMS), comprising the steps of administering a HDAC inhibitor to the subject, and administering a vinca alkaloid to the subject.

27. The method of claim 26 wherein the treating by administering a HDAC inhibitor and administering a vinca alkaloid is synergistic.

28. A therapeutic combination for treating rhabdomyosarcoma (RMS), comprising a therapeutically effective amount of a HDAC inhibitor and a therapeutically effective amount of a vinca alkaloid.

29. The therapeutic combination of claim 28 which is a synergistic combination.
